SEQUENCE LISTING (1 sequence)

SEQ ID NO: 1 - protein, 920 aa, Homo sapiens
MEVQLGLGRVYPRPPSKTYRGAFQNLFQSVREVIQNPGPRHPEAASAAPPGASLLLLQQQQQQQQQQQQQQQQQQQQQQQETSPRQQQQQQGEDGSPQAHRRGPTGYLVLDEEQQPSQPQSALECHPERGCVPEPGAAVAASKGLPQQLPAPPDEDDSAAPSTLSLLGPTFPGLSSCSADLKDILSEASTMQLLQQQQQEAVSEGSSSGRAREASGAPTSSKDNYLGGTSTISDNAKELCKAVSVSMGLGVEALEHLSPGEQLRGDCMYAPLLGVPPAVRPTPCAPLAECKGSLLDDSAGKSTEDTAEYSPFKGGYTKGLEGESLGCSGSAAAGSSGTLELPSTLSLYKSGALDEAAAYQSRDYYNFPLALAGPPPPPPPPHPHARIKLENPLDYGSAWAAAAAQCRYGDLASLHGAGAAGPGSGSPSAAASSSWHTLFTAEEGQLYGPCGGGGGGGGGGGGGGGGGGGGGGGEAGAVAPYGYTRPPQGLAGQESDFTAPDVWYPGGMVSRVPYPSPTCVKSEMGPWMDSYSGPYGDMRLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

(19) United States
(12) Patent Application Publication
(10) Pub. No.: US 2022/0184078 A1
(43) Pub. Date: Jun. 16, 2022

(54) METHODS OF TREATING PROSTATE CANCER

(71) Applicant: Arvinas Operations, Inc., New Haven, CT (US)

(72) Inventors: Sarah Deborah CHIRNOMAS, Woodbridge, CT (US); Richard Walter GEDRICH, Guilford, CT (US); Ronald PECK, Cheshire, CT (US); Ian Charles Anthony TAYLOR, Madison, CT (US)

(21) Appl. No.: 17/548,161

(22) Filed: Dec. 10, 2021

Related U.S. Application Data
(60) Provisional application No. 63/125,345, filed on Dec. 14, 2020.
(60) Provisional application No. 63/124,640, filed on Dec. 11, 2020.

(51) Int. Cl. A61K 31/506; A61K 9/00; A61K 31/58; A61P 35/00; A61K 31/496; A61K 31/501

(52) U.S. Cl. CPC A61K 31/506; A61K 9/0053; A61K 31/501; A61P 35/00; A61K 31/496; A61K 31/58

(57) ABSTRACT

The present application relates to treating and/or preventing prostate cancer, including metastatic and/or castrate-resistant prostate cancer, in a subject in need of treatment having particular somatic AR tumor biomarker status, comprising administering a compound of Formula (I),

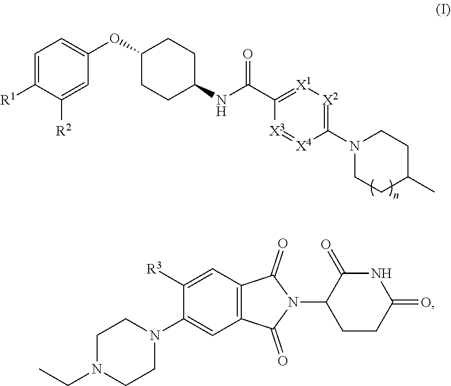

or a pharmaceutically acceptable salt, enantiomer, stereoisomer, solvate, polymorph, isotopic derivative, or prodrug thereof, wherein R¹, R², R³, X¹, X², X³, X⁴, and n are defined herein.

### CROSS-REFERENCE TO RELATED APPLICATIONS

This application claims priority to, and the benefit of, U.S. Provisional Application No. 63/124,640, filed Dec. 11, 2020, and U.S. Provisional Application No. 63/125,345, filed Dec. 14, 2020, the contents of which are incorporated herein by reference in their entirety.

### INCORPORATION BY REFERENCE OF SEQUENCE LISTING

This application contains a Sequence Listing which has been submitted in ASCII format via EFS-Web and is hereby incorporated by reference in its entirety. Said ASCII copy, created on Dec. 8, 2021, is named “ARVN-014-001US_ST25.txt” and is about 9 KB in size.

### BACKGROUND OF THE DISCLOSURE

Androgen Receptor (AR) belongs to a nuclear hormone receptor family that is activated by androgens, such as testosterone and dihydrotestosterone (Pharmacol. Rev. 2006, 58(4), 782-97; Vitam. Horn. 1999, 55:309-52.). In the absence of androgens, AR is bound by Heat Shock Protein 90 (Hsp90) in the cytosol. When an androgen binds AR, its conformation changes to release AR from Hsp90 and to expose the Nuclear Localization Signal (NLS). The latter enables AR to translocate into the nucleus where AR acts as a transcription factor to promote gene expression responsible for male sexual characteristics (Endocr. Rev. 1987, 8(1):1-28; Mol. Endocrinol. 2002, 16(10), 2181-7). AR deficiency leads to Androgen Insensitivity Syndrome, formerly termed testicular feminization.

While AR is responsible for development of male sexual characteristics, it is also a well-documented oncogene in certain forms of cancers including prostate cancers (Endocr. Rev. 2004, 25(2), 276-308). A commonly measured target gene of AR activity is the secreted Prostate Specific Antigen (PSA) protein. The current treatment regimen for prostate cancer involves inhibiting the androgen-AR axis by two methods. The first approach relies on reduction of androgens, while the second strategy aims to inhibit AR function (Nat. Rev. Drug Discovery, 2013, 12, 823-824). Despite the development of effective targeted therapies, most patients develop resistance and the disease progresses. An alternative approach for the treatment of prostate cancer involves eliminating the AR protein. Because AR is a critical driver of tumorigenesis in many forms of prostate cancers, its elimination should lead to a therapeutically beneficial response. There exists an ongoing need in the art for effective treatments for diseases, especially cancer, prostate cancer, and Kennedy's Disease. However, non-specific effects, and the inability to target and modulate certain classes of proteins altogether, such as transcription factors, remain as obstacles to the development of effective anti-cancer agents. As such, small molecule therapeutic agents that leverage or potentiate cereblon's substrate specificity and, at the same time, are “tunable” such that a wide range of protein classes can be targeted and modulated with specificity would be very useful as a therapeutic.

Over 70 different somatic missense AR tumor mutation have been identified in patients with prostate cancer (Gottlieb, B., Hum. Mutat. 2004, 23: 527-533). The majority of these AR tumor mutations reside in the ligand binding domain. Without being bound by theory, AR tumor mutations in the ligand binding domain result in decreased ligand specificity, thereby enabling AR to function independently of androgen. Such AR tumor mutations provide tumor cells with the capability to proliferate in androgen-depleted environments, and thus are selected for in response to therapies for prostate cancer that block or reduce androgen levels (e.g., luteinizing hormone-releasing hormone agonists). Accordingly, AR tumor mutations are observed with increased frequency in patients having advanced, androgen-independent tumors as compared to patients having early-stage prostate cancer (Taplin, M. E., et al. N. Engl. J. Med. (1995) 332: 1393-1398; Marcelli, M., et al. Cancer Res. (2000) 60: 944-949).

### SUMMARY OF THE DISCLOSURE

In one aspect, this application pertains to a method of treating prostate cancer in a subject in need thereof, wherein the prostate cancer comprises at least one somatic AR tumor mutation;
the method comprising administering to the subject a therapeutically effective amount of a compound of Formula (I),

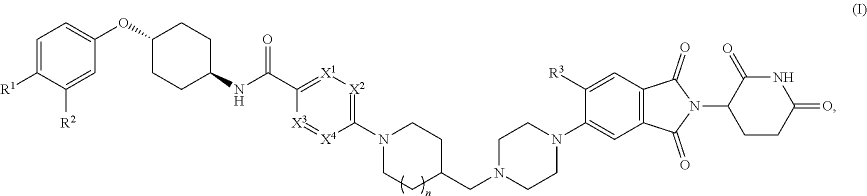

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH.

In some embodiments, the at least one somatic AR tumor mutation is selected from the group consisting H875X, Q825X, T878X, F877X, V716X, T878X, W742X, D891X, M750X, and S889X wherein “X” refers to any amino acid residue other than the wild type residue at that position.

In some embodiments, the at least one somatic AR tumor mutation is selected from the group consisting of T878A, H875Y, H875L, Q825E, W742C, W742L, F877L, T878S, V716M, D891H, M750V, M750T, and S889G.

In some embodiments, the prostate cancer comprises at least two somatic AR tumor mutations.

In some embodiments, the at least two somatic AR tumor mutations are selected from H875X, Q825X, T878X, F877X, V716X, T878X, and W742X, wherein “X” refers to any amino acid residue other than the wild type residue at that position.

In some embodiments, the at least two somatic AR tumor mutations are selected from: H875Y, H875L, Q825E, T878A, F877L, V716M, T878S, W742C, and W742L.

In some embodiments, the at least two somatic AR tumor mutations are selected from the following groups of mutations:

T878A, and H875Y;

H875L and Q825E;

T878A, F877L, and V716M;

T878A, M750T, and D891H;

T878S and H875Y;

T878A and T878S;

T878S and W742C; and

W742C and W742L.

In some embodiments, the prostate cancer comprises an amplification of the AR gene.

In some embodiments, the prostate cancer is castrate-resistant prostate cancer.

In some embodiments, the prostate cancer is metastatic prostate cancer.

In some embodiments, R¹ is CN and R² is chloro.

In some embodiments, R³ is hydrogen

In some embodiments, R³ is fluoro.

In some embodiments, n is 0.

In some embodiments, n is 1.

In some embodiments, each of X¹, X², X³, and X⁴ is CH.

In some embodiments, three of X¹, X², X³, and X⁴ are CH and the other is N.

In some embodiments, two of X¹, X², X³, and X⁴ are CH and the other two are N.

In some embodiments, the compound of Formula (I) is:

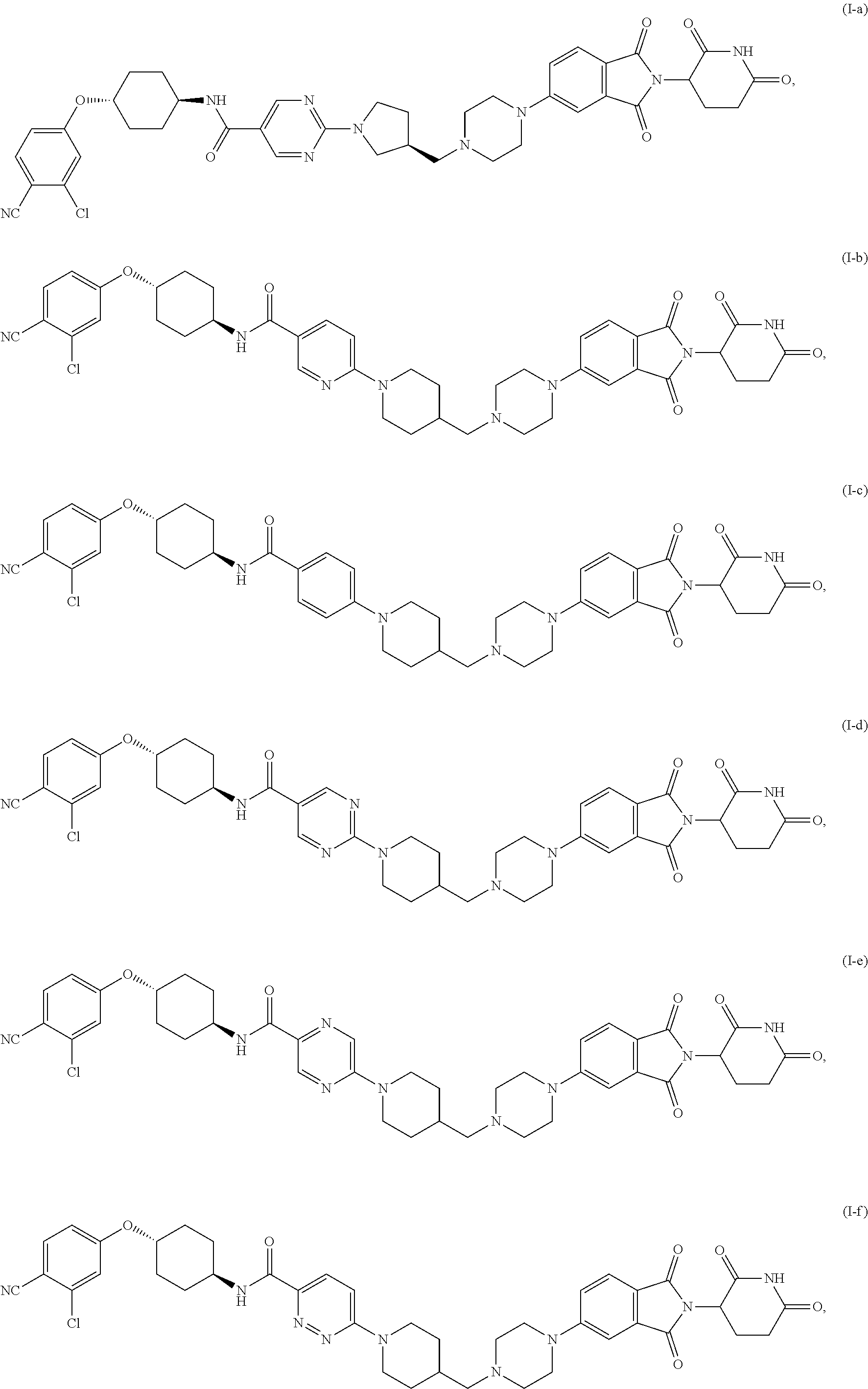

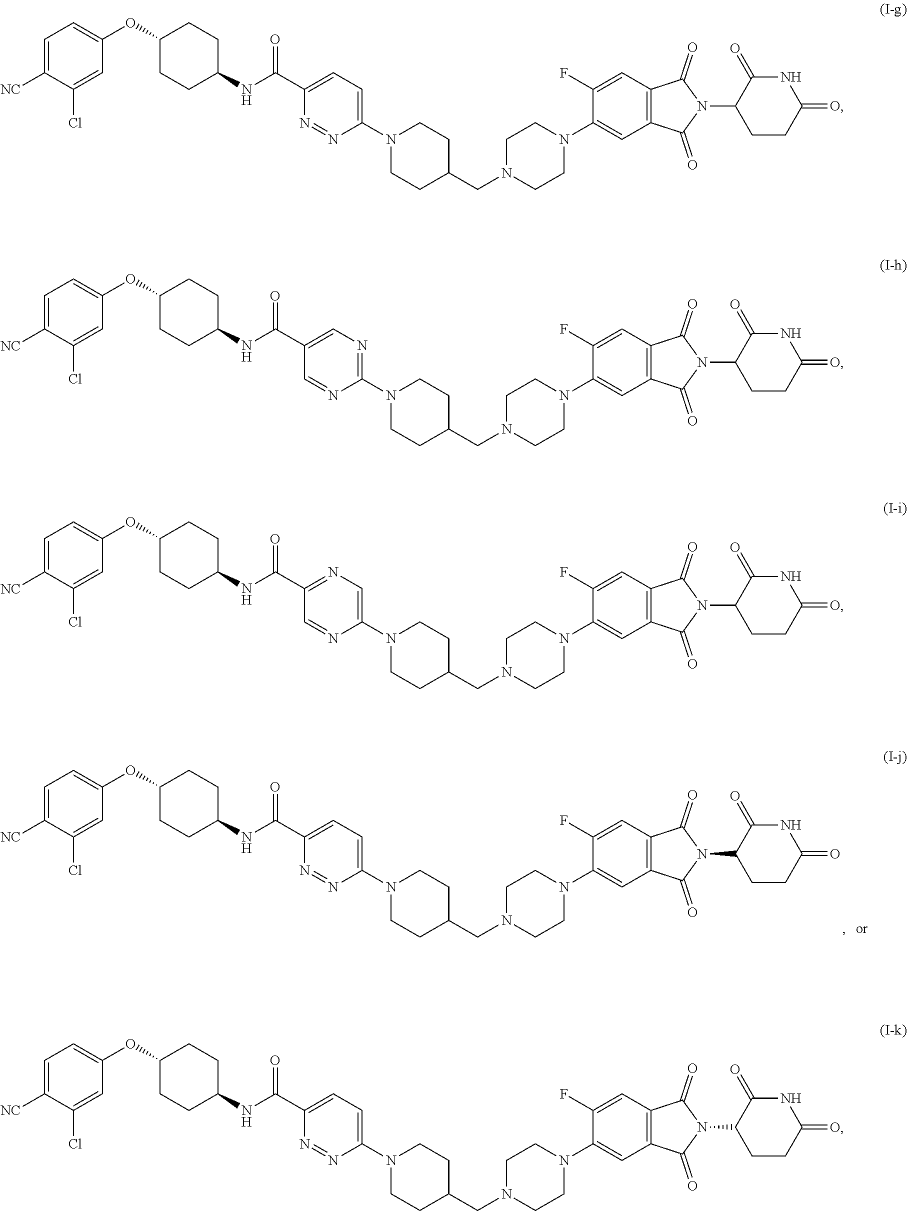

or a pharmaceutically acceptable salt thereof.

In some embodiments, the compound of Formula (I) is administered orally to the subject.

In some embodiments, the therapeutically effective amount of the compound of Formula (I) is administered to the subject once a day, twice a day, three times a day, or four times a day.

In some embodiments, the therapeutically effective amount of the compound of Formula (I) is administered to the subject once a day.

In some embodiments, the therapeutically effective amount of the compound of Formula (I) is administered to the subject all at once or is administered in two, three, or four unit doses.

In some embodiments, the therapeutically effective amount of the compound of Formula (I) is about 70 mg to about 1000 mg.

In some embodiments, the therapeutically effective amount of the compound of Formula (I) is about 100 mg to about 280 mg.

In some embodiments, the therapeutically effective amount of the compound of Formula (I) results in a mean day 15 AUC₀₋₂₄ of greater than about 4,500 ng*hr/mL, about 4,600 ng*hr/mL, about 4,700 ng*hr/mL, about 4,800 ng*hr/mL, about 4,900 ng*hr/mL, about 5,000 ng*hr/mL, about 5,100 ng*hr/mL, about 5,200 ng*hr/mL, about 5,300 ng*hr/mL, 5,400 ng*hr/mL, about 5,500 ng*hr/mL, about 5,600 ng*hr/mL, about 5,700 ng*hr/mL, about 5,800 ng*hr/mL, about 5,900 ng*hr/mL, or about 6,000 ng*hr/mL.

In some embodiments, the therapeutically effective amount of the compound of Formula (I) results in a mean day 15 AUC₀₋₂₄ of greater than about 4,500 ng*hr/mL and less than about 5,500 ng*hr/mL.

In some embodiments, the therapeutically effective amount of the compound of Formula (I) results in a mean day 15 Cₘₐₓ of greater than about 300 ng/mL and less than about 400 ng/mL.

In some embodiments, the therapeutically effective amount of the compound of Formula (I) results in a mean day 15 Cₘₐₓ of greater than about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, or about 380 ng/mL.

In some embodiments, the compound of Formula (I) is formulated as a tablet.

In some embodiments, the tablet comprises a compound of Formula (I) and, one or more excipients selected from the group consisting of the following: an emulsifier; a surfactant; a binder; a disintegrant; a glidant; and a lubricant.

In some embodiments, the subject is in a fed state.

In some embodiments, the subject is in a fasted state.

In one aspect, this application pertains to a method of treating prostate cancer in a subject in need thereof, comprising once a day, oral administration of a therapeutically effective amount of a compound of Formula (I), or a pharmaceutically acceptable salt thereof, wherein the compound of Formula (I) is selected from the group consisting of:

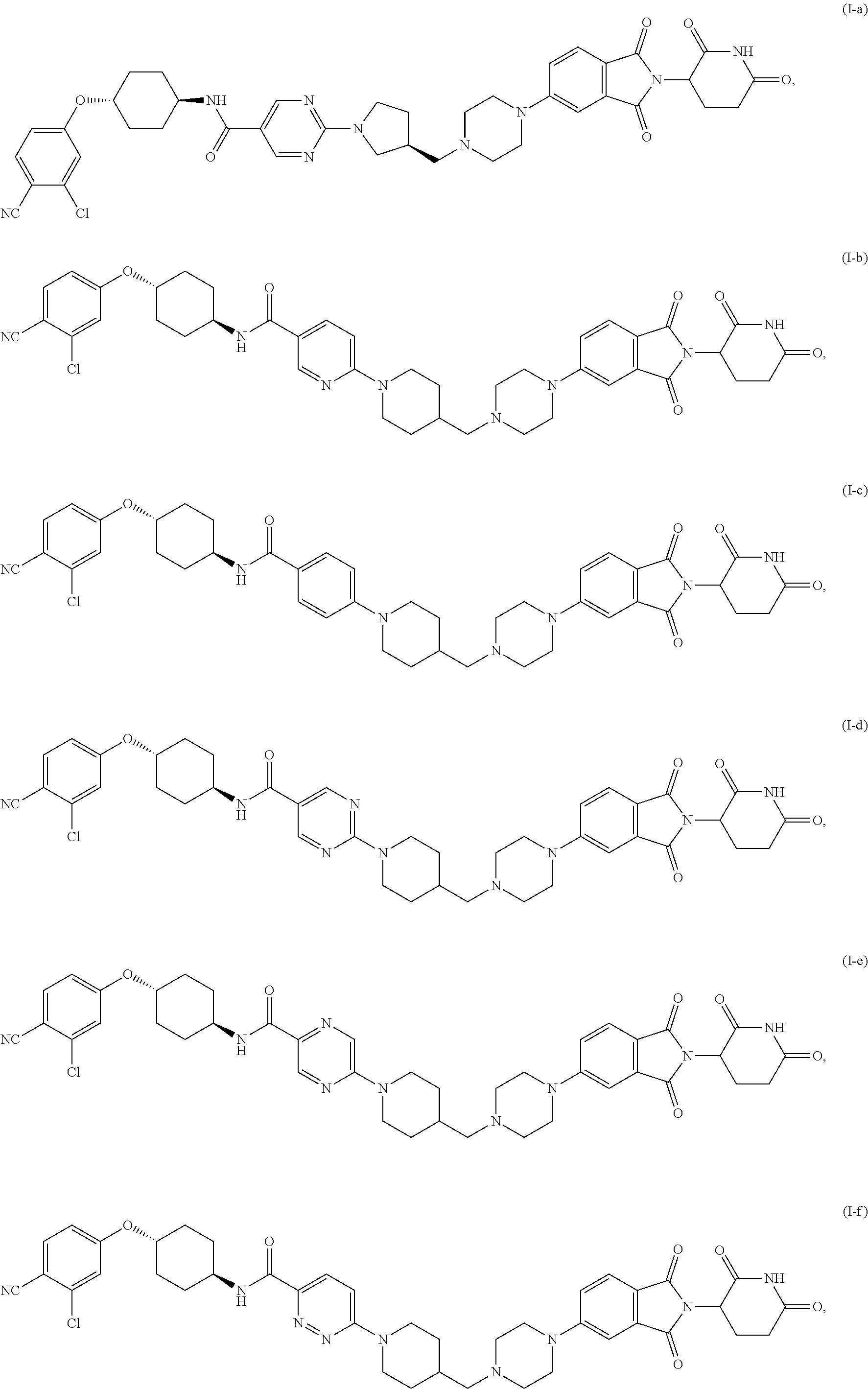

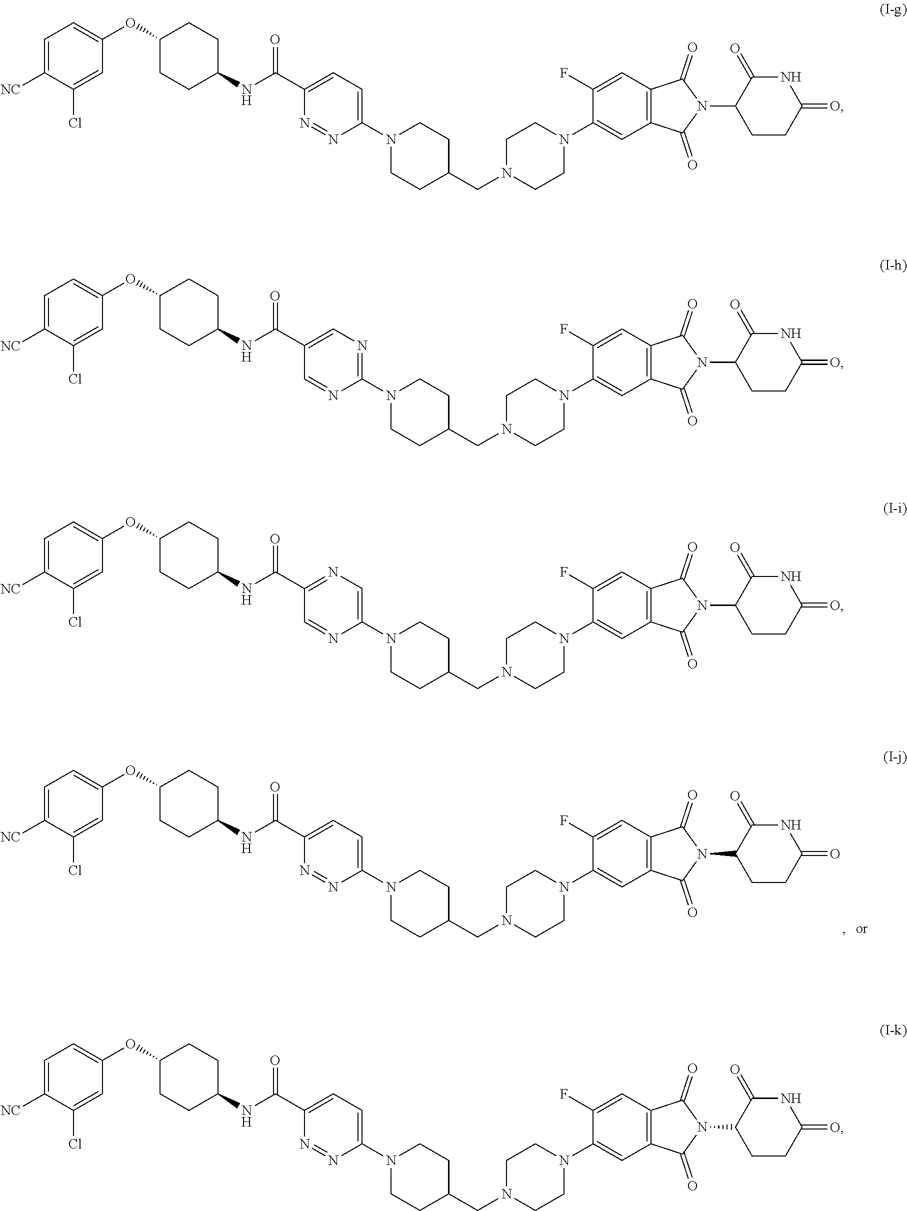

or a pharmaceutically acceptable salt thereof; wherein the prostate cancer comprises at least one somatic AR tumor mutation.

In some embodiments, the at least one somatic AR tumor mutation is selected from H875X, Q825X, T878X, F877X, V716X, T878X, W742X, D891X, M750X, and S889X wherein “X” refers to any amino acid residue other than the wild type residue at that position.

In some embodiments, the at least one somatic AR tumor mutation is selected from T878A, H875Y, H875L, Q825E, W742C, W742L, F877L, T878S, V716M, D891H, M750V, M750T, and S889G.

In some embodiments, the prostate cancer comprises at least two somatic AR tumor mutations.

In some embodiments, the at least two somatic AR tumor mutations are selected from H875X, Q825X, T878X, F877X, V716X, T878X, and W742X, wherein “X” refers to any amino acid residue other than the wild type residue at that position.

In some embodiments, the at least two somatic AR tumor mutations are selected from H875Y, H875L, Q825E, T878A, F877L, V716M, T878S, W742C, and W742L.

In some embodiments, the at least two somatic AR tumor mutations are selected from the following groups of mutations:

T878A and H875Y;

H875L and Q825E;

T878A, F877L, and V716M;

T878A, M750T, and D891H;

T878S and H875Y;

T878A and T878S;

T878S and W742C;

W742C and W742L.

In some embodiments, the prostate cancer comprises an amplification of the AR gene.

In some embodiments, the prostate cancer is castrate-resistant prostate cancer.

In some embodiments, the prostate cancer is metastatic prostate cancer.

In one aspect, this application pertains to a method of treating prostate cancer in a subpopulation of prostate cancer subjects, comprising:
selecting a subject with prostate cancer for treatment, wherein the subject's prostate cancer comprises at least one somatic AR mutation; and
administering a therapeutically effective amount of a compound of Formula (I),

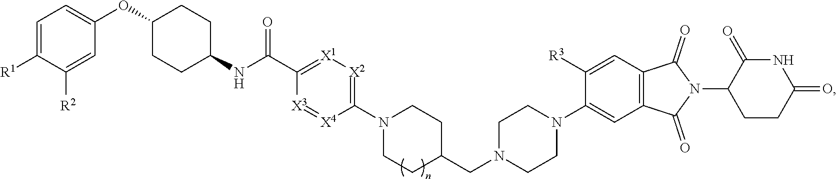

or a pharmaceutically acceptable salt thereof, to the subject, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH.

In some embodiments, the selected subject's prostate cancer comprises at least one somatic AR tumor mutation selected from H875X, Q825X, T878X, F877X, V716X, T878X, W742X, D891X, M750X, and S889X wherein “X” refers to any amino acid residue other than the wild type residue at that position.

In some embodiments, the selected subject's prostate cancer comprises at least one somatic AR tumor mutation selected from T878A, H875Y, H875L, Q825E, W742C, W742L, F877L, T878S, V716M, D891H, M750V, M750T, and S889G.

In some embodiments, the selected subject's prostate cancer comprises at least two somatic AR tumor mutations are selected from: H875X, Q825X, T878X, F877X, V716X, T878X, and W742X, wherein “X” refers to any amino acid residue other than the wild type residue at that position.

In some embodiments, the selected subject's prostate cancer comprises at least two somatic AR tumor mutations are selected from: H875Y, H875L, Q825E, T878A, F877L, V716M, T878S, W742C, and W742L.

In some embodiments, the selected subject's prostate cancer comprises at least two somatic AR tumor mutations selected from the following groups of mutations:

T878A, and H875Y;

H875L and Q825E;

T878A, F877L, and V716M;

T878S and H875Y;

T878S and W742C; and

W742C and W742L.

In some embodiments, the somatic AR tumor mutation of the prostate cancer in the selected subject is determined by ctDNA analysis, fluorescent in situ hybridization, immunohistochemistry, PCR analysis, or sequencing.

In some embodiments, the somatic AR tumor mutation of the prostate cancer in the selected subject is determined in a blood sample derived from the subject.

In some embodiments, the somatic AR tumor mutation of the prostate cancer in the selected subject is determined in a solid biopsy derived from the tumor of the subject.

In some embodiments, the compound of Formula (I) is selected from the group consisting of:

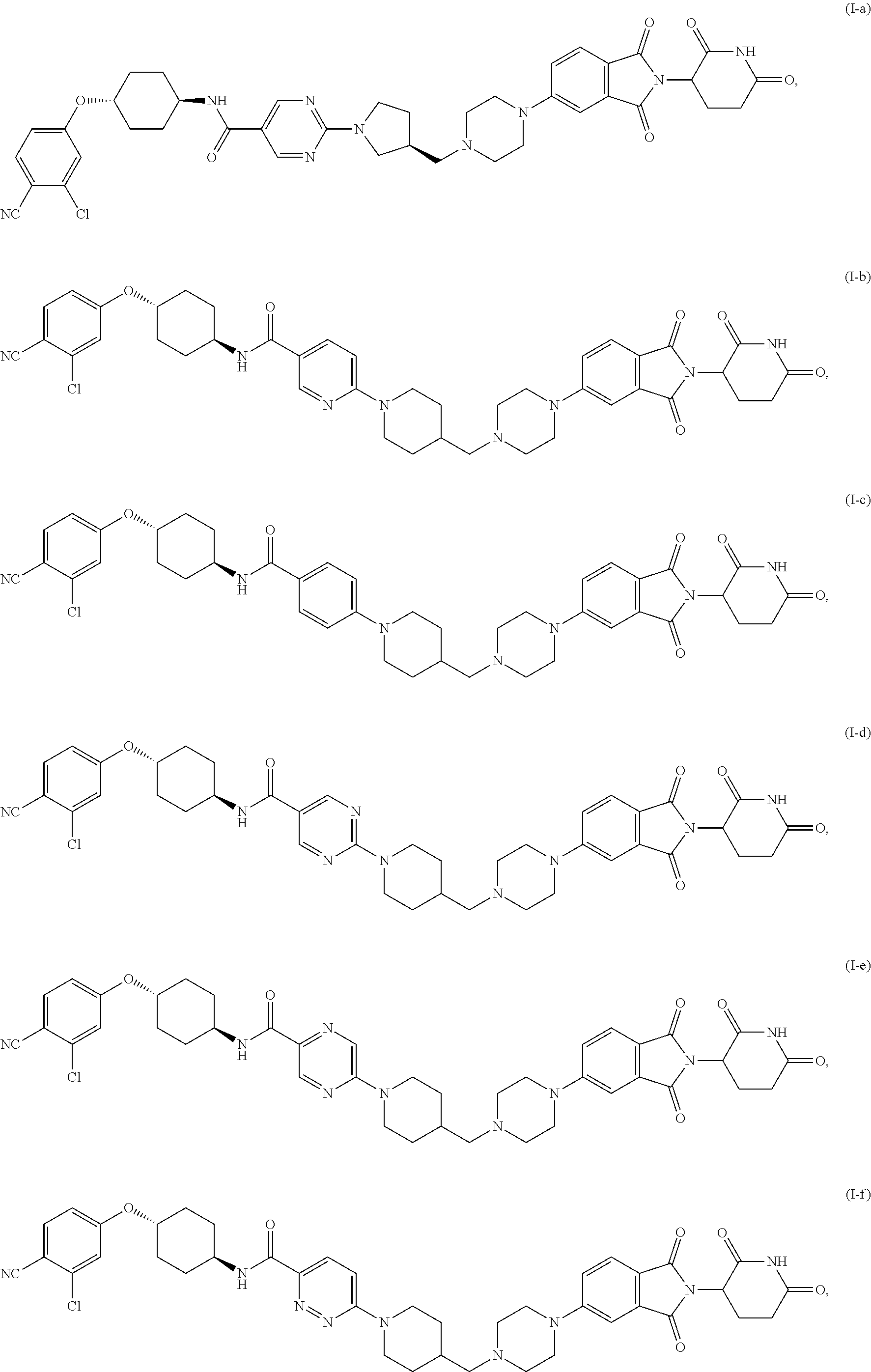

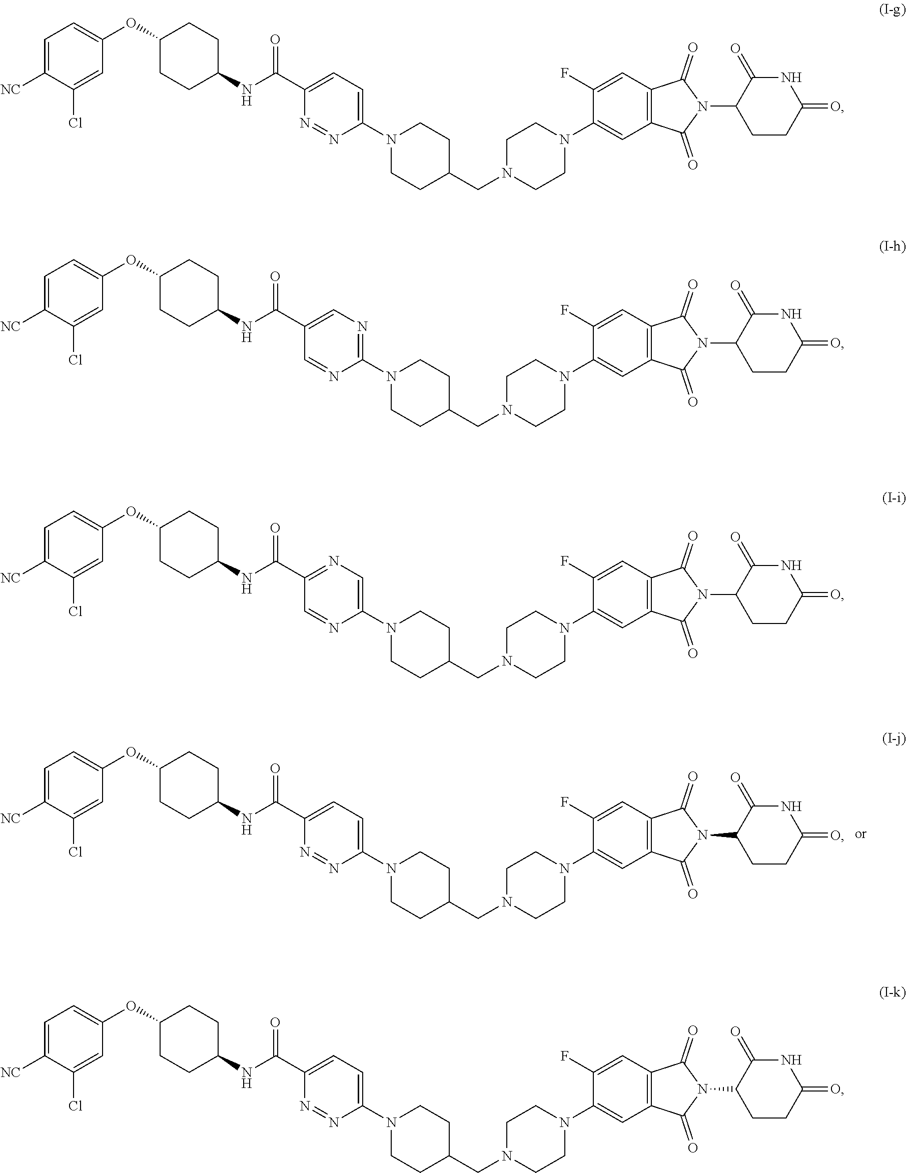

or a pharmaceutically acceptable salt thereof.

In some embodiments, the compound of Formula (I) is

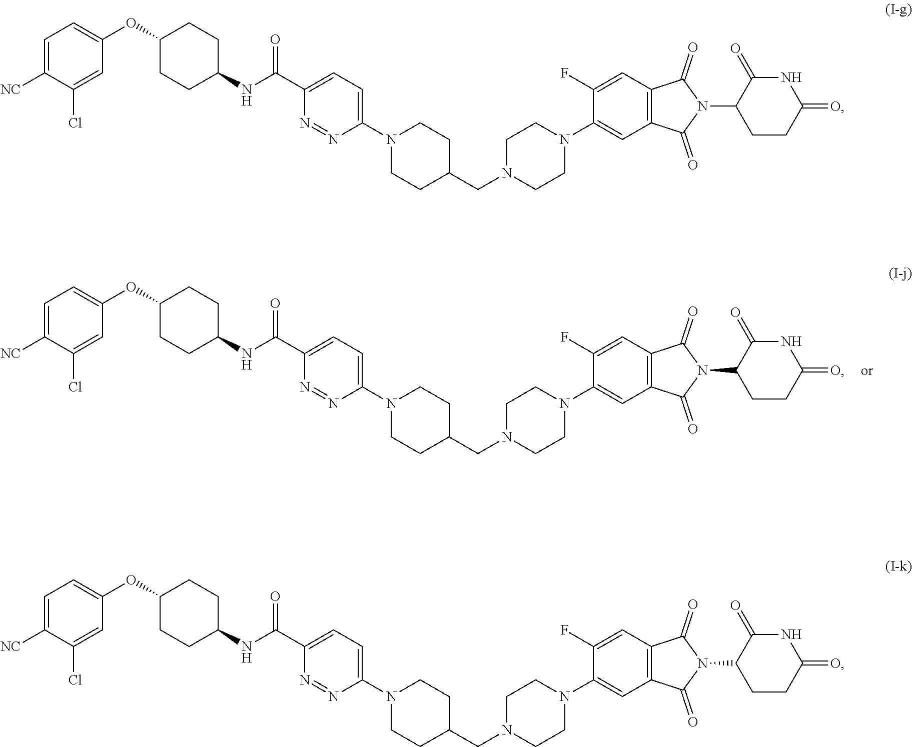

or a pharmaceutically acceptable salt thereof.

In some embodiments, the compound of Formula (I) is

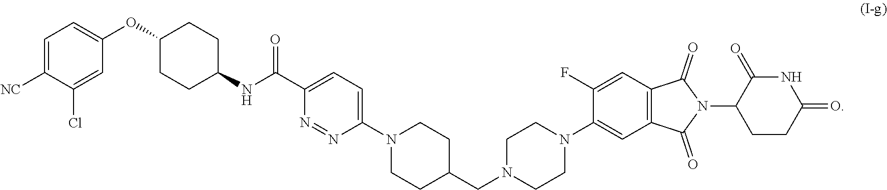 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

In some embodiments, the prostate cancer is castrate-resistant prostate cancer.

In some embodiments, the prostate cancer is metastatic prostate cancer.

In some embodiments, the administration of at least one additional anti-cancer agent.

In some embodiments, the additional anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

In some embodiments, the additional anti-cancer agent is selected from the group consisting of pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

In some embodiments, the compound of Formula (I) and the additional anti-cancer agent are administered to the subject simultaneously or in temporal proximity.

In one aspect, this application pertains to a compound of Formula (I),

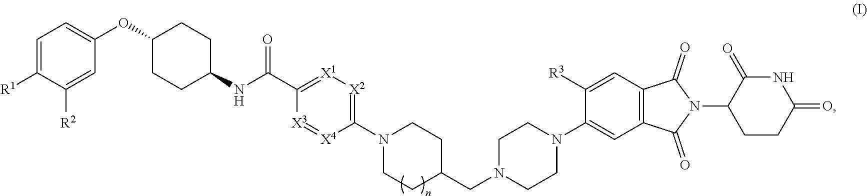

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
for use in the treatment of prostate cancer in a subject in need thereof, wherein the prostate cancer comprises at least one somatic AR tumor mutation.

In one aspect, this application pertains to a compound of Formula (I),

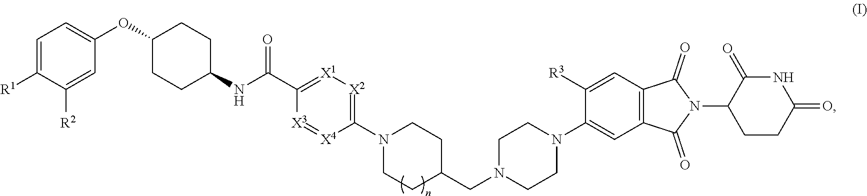

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
for use in a method of treating prostate cancer in a subject in need thereof, wherein the prostate cancer comprises at least one somatic AR tumor mutation; the method comprising administering to the subject between about 35 mg and about 1000 mg of the compound of Formula (I).

In one aspect, this application pertains to a compound of Formula (I),

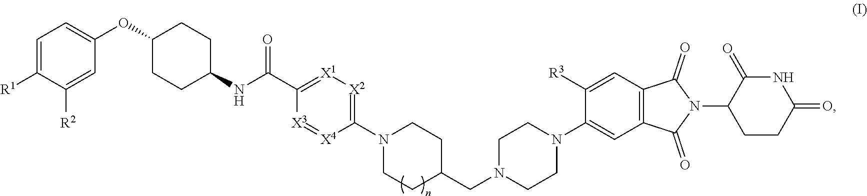

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
   provided that at least two of X¹, X², X³, and X⁴ are CH;
   for use in a method of treating prostate cancer in a subject in need thereof, wherein the prostate cancer comprises at least one somatic AR tumor mutation; the method comprising once a day, oral administration of a therapeutically effective amount of the compound of Formula (I); and wherein the therapeutically effective amount of the compound of Formula (I) is about 35 mg to about 1000 mg.

In one aspect, this application pertains to a compound of Formula (I),

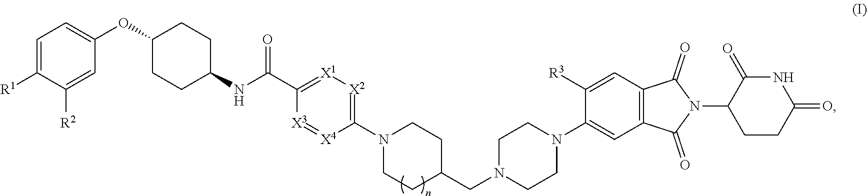

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
for use in a method of treating prostate cancer in a subpopulation of prostate cancer subjects in need thereof, wherein the prostate cancer comprises at least one somatic AR tumor mutation; the method comprising:
selecting a subject with prostate cancer for treatment, wherein the subject's prostate cancer comprises at least one somatic AR mutation; and administering a therapeutically effective amount of a compound of Formula (I).

In one aspect, this application pertains to a compound of Formula (I),

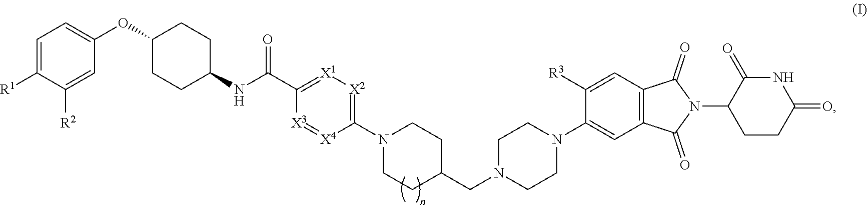

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH;
for use in the manufacture of a medicament for the treatment of prostate cancer in a subject in need thereof, wherein the prostate cancer comprises at least one somatic AR tumor mutation.

In some embodiments, the at least one somatic AR tumor mutation is selected from the group consisting H875X, Q825X, T878X, F877X, V716X, T878X, W742X, D891X, M750X, and S889X wherein “X” refers to any amino acid residue other than the wild type residue at that position.

In some embodiments, the at least one somatic AR tumor mutation is selected from the group consisting of T878A, H875Y, H875L, Q825E, W742C, W742L, F877L, T878S, V716M, D891H, M750V, M750T, and S889G.

In some embodiments, the prostate cancer comprises at least two somatic AR tumor mutations.

In some embodiments, the at least two somatic AR tumor mutations are selected from H875X, Q825X, T878X, F877X, V716X, T878X, and W742X, wherein “X” refers to any amino acid residue other than the wild type residue at that position.

In some embodiments, the at least two somatic AR tumor mutations are selected from: H875Y, H875L, Q825E, T878A, F877L, V716M, T878S, W742C, and W742L.

In some embodiments, the at least two somatic AR tumor mutations are selected from the following groups of mutations:

T878A, and H875Y;

H875L and Q825E;

T878A, F877L, and V716M;

T878A, M750T, and D891H;

T878S and H875Y;

T878A and T878S;

T878S and W742C; and

W742C and W742L.

In some embodiments, the prostate cancer comprises an amplification of the AR gene.

In some embodiments, the prostate cancer is castrate-resistant prostate cancer.

In some embodiments, the prostate cancer is metastatic prostate cancer.

In some embodiments, the compound of Formula (I) is:

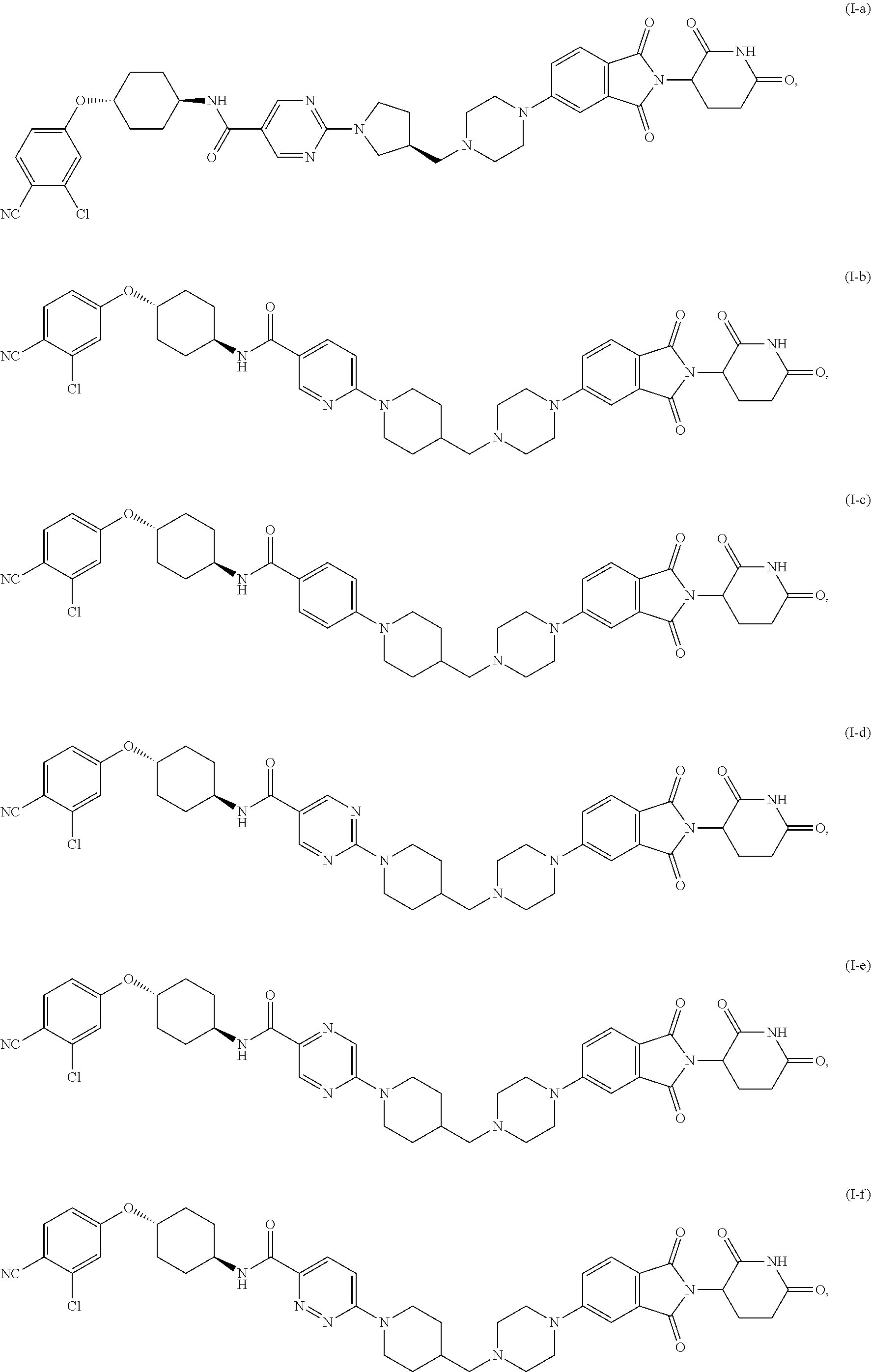

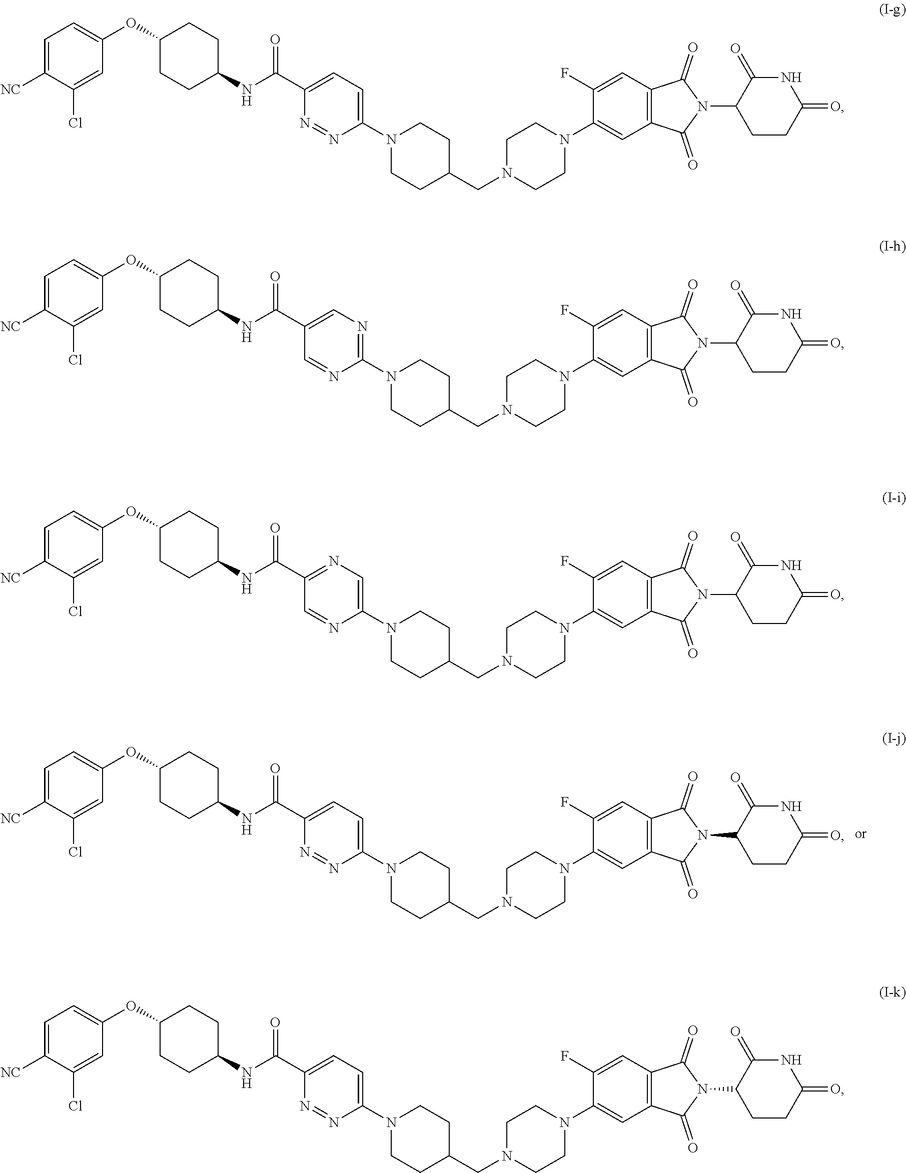

or a pharmaceutically acceptable salt thereof.

In some embodiments, the compound of Formula (I) is:

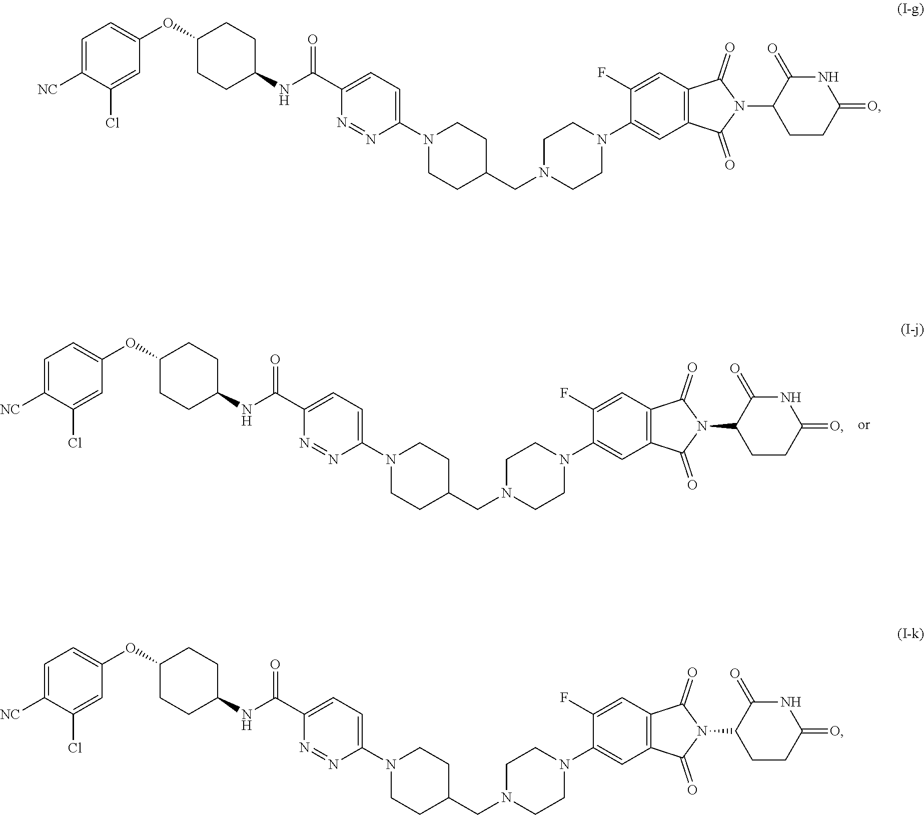

or a pharmaceutically acceptable salt thereof.

In some embodiments, the compound of Formula (I) is:

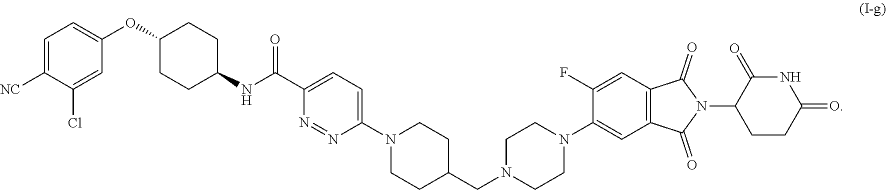 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

In some embodiments, the use or method of treating of prostate cancer in a subject in need thereof comprises the use at least one additional anti-cancer agent.

In some embodiments, the additional anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STA T inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

In some embodiments, the additional anti-cancer agent is selected from the group consisting of pemetrexed, ipilimumab, vorinostat, etoposide, gemcitabine, doxorubicin, vincstine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

In some embodiments, the compound of Formula (I) and the additional anti-cancer agent are administered to the subject simultaneously or in temporal proximity.

In one aspect, this application pertains to a kit comprising:

(a) compound of Formula (I-g):

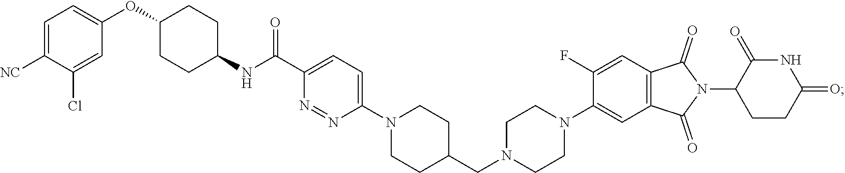 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

(b) an additional anti-cancer agent; and

(c) instructions for use.

In some embodiments, the additional anti-cancer agent in the kit is a FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, or chemical castration agent.

In some embodiments, the additional anti-cancer agent in the kit is pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, or zolendronate.

### BRIEF DESCRIPTION OF THE FIGURES

FIG. 1 is a dose-response curve comparing the in vitro inhibitory effect of VCaP proliferation of Compound (I-g) with enzalutamide.

FIG. 2 is Western Blot experiment that shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

FIG. 3 is series of line graphs summarizing animal experiments performed in a castrated VCaP xenograft model. Compound (I-g) was administered orally, once daily at doses of 0.1 mg/kg (mpk), 0.3 mg/kg, 1 mg/kg, and 3 mg/kg. Enzalutamide (20 mg/kg) and vehicle were also used as control groups.

FIG. 4 is series of line graphs summarizing animal experiments performed in an intact (non-castrated) VCaP xenograft model. Compound (I-g) was administered orally, once daily at doses of 1 mg/kg, 3 mg/kg, and 10 mg/kg. Enzalutamide (20 mg/kg) and vehicle were also used as control groups.

FIG. 5 is series of line graphs summarizing animal experiments performed in an enzalutamide resistant VCaP xenograft model. Compound (I-g) was administered orally, once daily at doses of 3 mg/kg and 10 mg/kg. Enzalutamide (20 mg/kg) and vehicle were also used as control groups.

FIG. 6 is Western Blot experiment that shows the reduction of AR in enzalutamide-resistant VCaP tumors in response to dosing with Compound (I-g) at 10 mg/kg and 3 mg/kg (oral, once daily).

FIG. 7 is a series of line graphs which provides a representation of the mean concentrations of Compound (I-g) over a 24 hour time period after dosing on day 15 for all three tested doses (35 mg/day, 70 mg/day, and 140 mg/day, oral administration).

FIG. 8 is a bar graph (aka, waterfall plot) showing the best percent change in Prostate-Specific Antigen (PSA) test results in 20 patients with metastatic castration resistant prostate cancer (mCRPC) receiving Compound (I-g). Each bar represents the best percent change in plasma PSA from pre-treatment levels of a single patient. Patients received either 35 mg/day, 70 mg/day, 140 mg/day, or 280 mg/day of Compound (I-g), as indicated in the legend.

FIG. 9 is a bar graph (aka, waterfall plot) showing best percent change in Prostate-Specific Antigen (PSA) test results in 12 patients with mCRPC receiving ≥140 mg daily dose of Compound (I-g), as well as the molecular status of the AR gene or protein present in circulating tumor DNA or circulating tumor cells, respectively, isolated from each patient. Each bar represents the best percent change in plasma PSA from pre-treatment levels of a single patient. AR-V7 is a splice variant of AR. Amplif. refers to amplification of the AR gene.

FIG. 10 summarizes the key features of one patient (“patient 19”) who received a 140 mg/day dose of Compound (I-g). This patient corresponds with the second bar from right in both FIG. 8 and FIG. 9.

FIG. 11A summarizes the key features of one patient (“patient 20”) who received a 140 mg/day dose of Compound (I-g). This patient corresponds with rightmost bar in FIG. 8 and FIG. 9. FIG. 11B shows a CT scan of the patient 20's tumor prior to treatment. FIG. 11C shows a CT scan of the patient 20's tumor after 4 cycles, showing the RECIST response.

FIG. 12 is a representation of the Mean Day 15 AUC₀₋₂₄ (ng*hr/mL) of Compound (I-g) over a 24 hour time period after dosing on day 15 for all four tested doses (35 mg/day, 70 mg/day, 140 mg/day, and 280 mg/day, oral administration).

FIG. 13 is a series of line graphs which provides a representation of the mean concentrations of Compound (I-g) over a 24 hour time period after dosing on day 15 for all four tested doses (in order from lowest to highest on the y-axis—35 mg/day, 70 mg/day, 140 mg/day, and 280 mg/day, oral administration).

FIG. 14 is a waterfall plot showing the best percent change in Prostate-Specific Antigen (PSA) test results in 28 patients with mCRPC receiving Compound (I-g), as well as the molecular status of the AR gene present in circulating tumor DNA or circulating tumor cells, respectively, determined for each patient. Each bar represents the best percent change in plasma PSA from pre-treatment levels of a single patient. AR-V7 is a splice variant of AR. Amplif. refers to amplification of the AR gene.

FIG. 15 is a waterfall plot showing the best percent change in Prostate-Specific Antigen (PSA) test results in 5 patients with mCRPC with AR T878/H875 mutations after receiving Compound (I-g), as well as the molecular status of the AR gene present in circulating tumor DNA or circulating tumor cells, respectively, isolated from each patient. Each bar represents the best percent change in plasma PSA from pre-treatment levels of a single patient. AR-V7 is a splice variant of AR.

### SEQUENCE LISTING

All references to amino acid mutations in the Androgen Receptor are numbered relative to SEQ ID NO: 1, which is provided below:

| | |
|---|---|
| 1 | mevqlglgrv yprppsktyr gafqnlfqsv reviqnpgpr hpeaasaapp gasllllqqq |
| 61 | qqqqqqqqqq qqqqqqqqqq etsprqqqqq qgedgspqah rrgptgylvl deeqqpsqpq |
| 121 | salechperg cvpepgaava askglpqqlp appdeddsaa pstlsllgpt fpglsscsad |
| 181 | lkdilseast mqllqqqqqe aysegsssgr areasgapts skdnylggts tisdnakelc |
| 241 | kavsvsmglg vealehlspg eqlrgdcmya pllgvppavr ptpcaplaec kgsllddsag |
| 301 | kstedtaeys pfkggytkgl egeslgcsgs aaagssgtle lpstlslyks galdeaaayq |
| 361 | srdyynfpla lagppppppp phpharikle npldygsawa aaaaqcrygd laslhgagaa |
| 421 | gpgsgspsaa assswhtlft aeegqlygpc gggggggggg gggggggggg gggeagavap |
| 481 | ygytrppqgl agqesdftap dvwypggmvs rvpypsptcv ksemgpwmds ysgpygdmrl |
| 541 | etardhvlpi dyyfppqktc licgdeasgc hygaltcgsc kvffkraaeg kqkylcasrn |
| 601 | dctidkfrrk ncpscrlrkc yeagmtlgar klkklgnlkl qeegeasstt spteettqkl |
| 661 | tvshiegyec qpiflnvlea iepgvvcagh dnnqpdsfaa llsslnelge rqlvhvvkwa |
| 721 | kalpgfrnlh vddqmaviqy swmglmvfam gwrsftnvns rmlyfapdlv fneyrmhksr |
| 781 | mysqcvrmrh lsqefgwlqi tpqeflcmka lllfsiipvd glknqkffde lrmnyikeld |
| 841 | riiackrknp tscsrrfyql tklldsvqpi arelhqftfd llikshmvsv dfpemmaeii |
| 901 | svqvpkilsg kvkpiyfhtq |

### DETAILED DESCRIPTION

### Definitions

“Halogen” or “halo” refers to fluorine (F), chlorine (Cl), bromine (Br), or iodine (I).

“C₁-C₆ alkyl” refers to a straight or branched chain saturated hydrocarbon containing 1-6 carbon atoms. Examples of a (C₁-C₆) alkyl group include, but are not limited to, methyl, ethyl, propyl, butyl, pentyl, hexyl, isopropyl, isobutyl, sec-butyl, tert-butyl, isopentyl, neopentyl, and isohexyl.

“Pharmaceutically acceptable salt”, as used herein with respect to a compound of Formula (I), means a salt form of a compound of Formula (I) as well as hydrates of the salt form with one or more water molecules present. Such salt and hydrated forms retain the biological activity of a compound of Formula (I) and are not biologically or otherwise undesirable, i.e., exhibit minimal, if any, toxicological effects. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

The term “isomer” refers to salts and/or compounds that have the same composition and molecular weight but differ in physical and/or chemical properties. The structural difference may be in constitution (geometric isomers) or in the ability to rotate the plane of polarized light (stereoisomers). With regard to stereoisomers, the salts of a compound of Formula (I) may have one or more asymmetric carbon atom and may occur as racemates, racemic mixtures and as individual enantiomers or diastereomers.

The compounds of Formula (I) may exist in unsolvated as well as solvated forms such as, for example, hydrates.

“Solvate” means a solvent addition form that contains either a stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds of the invention.

When a compound is crystallized from a solution or slurry, it can be crystallized in a different arrangement lattice of spaces (this property is called “polymorphism”) to form crystals with different crystalline forms, each of which is known as “polymorphs”. “Polymorph”, as used herein, refers to a crystal form of a compound of Formula (I) where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid state stability, etc.

“Isotopic derivative”, as referred to herein, relates to a compound of Formula (I) that is isotopically enriched or labelled (with respect to one or more atoms of the compound) with one or more stable isotopes. Thus, in this application, the compounds of Formula (I) include, for example, compounds that are isotopically enriched or labelled with one or more atoms such as deuterium.

The term “pharmaceutically acceptable prodrugs” as used herein refers to those prodrugs of the compounds of Formula (I) which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present invention.

“Prodrug”, as used herein means a compound which is convertible in vivo by metabolic means (e.g., by hydrolysis) to afford any compound delineated by the formulae of the instant invention. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs, Textbook of Drug Design and Development, Chapter 5, 1 13-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

This invention also encompasses pharmaceutical compositions containing, and methods of treating disorders through administering, pharmaceutically acceptable prodrugs of compounds of the invention. For example, compounds of the invention having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the invention. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. Free amines can also be derivatized as amides, sulfonamides or phosphonamides. All of these prodrug moieties may incorporate groups including but not limited to ether, amine and carboxylic acid functionalities. Combinations of substituents and variables envisioned by this invention are only those that result in the formation of stable compounds.

Metastatic prostate cancer, or metastases, refers to prostate cancer that has spread beyond the prostate to other parts of the body, e.g., bones, lymph nodes, liver, lungs, brain.

Castrate-resistant prostate cancer or castration-resistant prostate cancer (or prostate cancer that is castrate- or castration-resistant) is a type of prostate cancer that keeps growing even when the amount of testosterone in the body is reduced to very low levels.

Metastatic, castrate-resistant prostate cancer is a type of prostate cancer that has metastasized and continues to grow even when the amount of testosterone in the body is reduced to very low levels.

As used herein, “treating” describes the management and care of a subject for the purpose of combating a disease, condition, or disorder and includes decreasing or alleviating the symptoms or complications, or eliminating the disease, condition or disorder.

As used herein, “preventing” describes stopping the onset of the symptoms or complications of the disease, condition or disorder.

“Administration” refers to introducing an agent, such as a compound of Formula (I) into a subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug is administering the drug to the patient.

“Anti-cancer agent”, as used herein, is used to describe an agent effective at treating cancer, or a therapeutic agent administered concurrently with an anti-cancer agent (e.g., palonosetron), with which may be co-administered and/or co-formulated with a compound of Formula (I) to treat cancer, and the side effects associated with the cancer treatment.

In some embodiments, the additional anti-cancer agent is selected from any of the following: FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

In some embodiments, the additional anti-cancer agent is selected from any of the following: pemetrexed, ipilimumab, vorinostat, etoposide, gemcitabine, doxorubicin, vincristine, temozolomide, capecitabine, irinotecan, tamoxifen, anastrazole, exemestane, letrozole, DES (diethylstilbestrol), estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

Abiraterone acetate is a commercially available drug for the treatment of metastatic castration-resistant prostate cancer developed by Janssen and sold under the brand name Zytiga®.

“Therapeutically effective amount”, as used herein means an amount of the free base of a compound of Formula (I) that is sufficient to treat, ameliorate, or prevent a specified disease (e.g., prostate cancer), disease symptom, disorder or condition, or to exhibit a detectable therapeutic or inhibitory effect. The effect can be detected by any assay method known in the art. The effective amount for a particular subject may depend upon the subject's body weight, size, and health; the nature and extent of the condition; and whether additional therapeutics are to be administered to the subject. Therapeutically effective amounts for a given situation can be determined by routine experimentation that is within the skill and judgment of the clinician.

“Cₘₐₓ”, as used herein, refers to the observed maximum (peak) plasma concentration of a specified compound in the subject after administration of a dose of that compound to the subject.

“AUC”, as used herein, refers to the total area under the plasma concentration-time curve, which is a measure of exposure to a compound of interest, and is the integral of the concentration-time curve after a single dose or at steady state. AUC is expressed in units of ng*H/mL (ng×H/mL).

“AUCₜₐᵤ”, as used herein, refers to the AUC from 0 hours to the end of a dosing interval.

“AUC₀₋₂₄” means the AUC from 0 hours to 24 hours after administration of a single dose.

“Controlled release” or “CR” as used herein with respect to an oral dosage form of the disclosure means that a compound of Formula (I) is released from the dosage form according to a pre-determined profile that may include when and where release occurs after oral administration and/or a specified rate of release over a specified time period.

“Controlled release agent” as used herein with respect to an oral dosage form of the disclosure refers to one or more substances or materials that modulate release of a compound of Formula (I) from the dosage form. Controlled release agents may be materials which are organic or inorganic, naturally occurring or synthetic, such as polymeric materials, triglycerides, derivatives of triglycerides, fatty acids and salts of fatty acids, talc, boric acid and colloidal silica.

“Enteric coating” as used herein with respect to a dosage form of the disclosure refers to a pH-dependent material that surrounds a core comprising a compound of Formula (I) and which remains substantially intact in the acid environment of the stomach, but which dissolves in the pH environment of the intestines.

“Gastro-resistant” or “GR” as applied to a CR oral dosage form described herein means that release of a compound of Formula (I) in the stomach of a subject shall not exceed 5%, 2.5%, 1% or 0.5% of the total amount of the compound of Formula (I) in the dosage form.

“Oral dosage form” as used herein refers to a pharmaceutical drug product that contains a specified amount (dose) of a compound of Formula (I) as the active ingredient, or a pharmaceutically acceptable salt and/or solvate thereof, and inactive components (excipients), formulated into a particular configuration that is suitable for oral administration, such as a tablet or capsule. In some embodiments, the compositions are in the form of a tablet that can be scored.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

Abiraterone acetate is a commercially available drug for the treatment of metastatic castration-resistant prostate cancer developed by Janssen and sold under the brand name Zytiga®.

The term “about” as part of a quantitative expression such as “about X”, includes any value that is 10% higher or lower than X, and also includes any numerical value that falls between X−10% and X+10%. Thus, for example, a weight of about 40 g includes a weight of between 36 to 44 g. When used herein to denote amino acid residues in the AR, the term “about” means any amino acid residue that is within 5 amino acid residues of what is specified. For example, when referring to a contiguous stretch of amino acid residues extending from about amino acid residue 560 to about amino acid residue 624 of the AR, this refers to a contiguous stretch of amino acid residues extending from amino acid residue 555, 556, 557, 558, 559, 560, 561, 562, 563, 564, or 565, to amino acid residue 619, 620, 621, 622, 623, 624, 625, 626, 627, 628, or 629 of the AR of SEQ ID NO: 1. In some embodiments, the term “about” means any amino acid residue that is within 3 amino acid residues of what is specified. In some embodiments, the term “about” means any amino acid residue that is within 1 amino acid residue of what is specified.

“Comprising” or “comprises” as applied to a particular dosage form, composition, use, method or process described or claimed herein means that the dosage form, composition, use, method, or process includes all of the recited elements in a specific description or claim, but does not exclude other elements. “Consists essentially of” and “consisting essentially of” means that the described or claimed composition, dosage form, method, use, or process does not exclude other materials or steps that do not materially affect the recited physical, pharmacological, pharmacokinetic properties or therapeutic effects of the composition, dosage form, method, use, or process. “Consists of” and “consisting of” means the exclusion of more than trace elements of other ingredients and substantial method or process steps.

“Fasted condition” or “fasted state” as used to describe a subject means the subject has not eaten for at least 4 hours before a time point of interest, such as the time of administering a compound of Formula (I). In an embodiment, a subject in the fasted state has not eaten for at least any of 6, 8, 10 or 12 hours prior to administration of a compound of Formula (I).

“Fed condition” or “fed state” as used to describe a subject herein means the subject has eaten less than 4 hours before a time point of interest, such as the time of administering a compound of Formula (I). In an embodiment, a subject in the fed state has not eaten for at least any of 3, 2, 1 or 0.5 hours prior to administration of a compound of Formula (I).

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The terms “patient” and “subject” are used interchangeably herein, and refer to a mammal, e.g., a human, mouse, rat, guinea pig, dog, cat, horse, cow, pig, or non-human primate, such as a monkey, chimpanzee, baboon or rhesus.

In some embodiments, the subject is a human.

In some embodiments, the subject is a human who has been diagnosed with prostate cancer.

In some embodiments, the subject is a human who has been diagnosed with metastatic prostate cancer.

In some embodiments, the subject is a human who has been diagnosed with castrate-resistant prostate cancer.

In some embodiments, the subject is a human who has been diagnosed with metastatic, castrate-resistant prostate cancer.

### Compounds of Formula (I)

In one aspect, the application pertains to the methods of treating and/or preventing cancer comprising the administration of a compound of Formula (I) to subject in need thereof. In one aspect, the application pertains to the use of a compound of Formula (I) in the treatment and/or prevention of prostate cancer. In one aspect, the application pertains to the use of a compound of Formula (I) in the manufacture of a medicament for the treatment and/or prevention of prostate cancer.

As referred to herein, a compound of Formula (I) refers to a compound with the following structure:

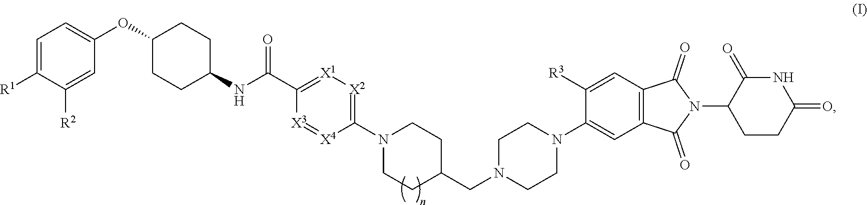

or a pharmaceutically acceptable salt, enantiomer, stereoisomer, solvate, polymorph, isotopic derivative, or prodrug thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1.

In some embodiments, R¹ is hydrogen.

In some embodiments, R¹ is CN.

In some embodiments, R¹ is C₁-C₆ alkyl.

In some embodiments, R² is hydrogen.

In some embodiments, R² is halo. In some embodiments, R² is F. In some embodiments, R² is Cl. In some embodiments, R² is Br. In some embodiments, R² is I.

In some embodiments, R² is C₁-C₆ alkyl.

In some embodiments, R³ is hydrogen.

In some embodiments, R³ is halo. In some embodiments, R³ is F. In some embodiments, R³ is Cl. In some embodiments, R³ is Br. In some embodiments, R³ is I.

In some embodiments, at least one of X¹, X², X³, and X⁴ is CH.

In some embodiments, at least two of X¹, X², X³, and X⁴ are CH.

In some embodiments, at least three of X¹, X², X³, and X⁴ are CH.

In some embodiments, each of X¹, X², X³, and X⁴ is CH.

In some embodiments, X¹, X², and X³ are each CH, and X⁴ is N.

In some embodiments, X¹, X², and X⁴ are each CH, and X³ is N.

In some embodiments, X¹, X³, and X⁴ are each CH, and X² is N.

In some embodiments, X², X³, and X⁴ are each CH, and X¹ is N.

In some embodiments, X¹ and X² are each CH, and X³ and X⁴ are each N.

In some embodiments, X¹ and X³ are each CH, and X² and X⁴ are each N.

In some embodiments, X¹ and X⁴ are each CH, and X² and X³ are each N.

In some embodiments, X² and X³ are each CH, and X¹ and X⁴ are each N.

In some embodiments, X² and X⁴ are each CH, and X¹ and X³ are each N.

In some embodiments, X³ and X⁴ are each CH, and X¹ and X² are each N.

In some embodiments, n is 0.

In some embodiments, n is 1.

In some embodiments, the compound of Formula (I) is

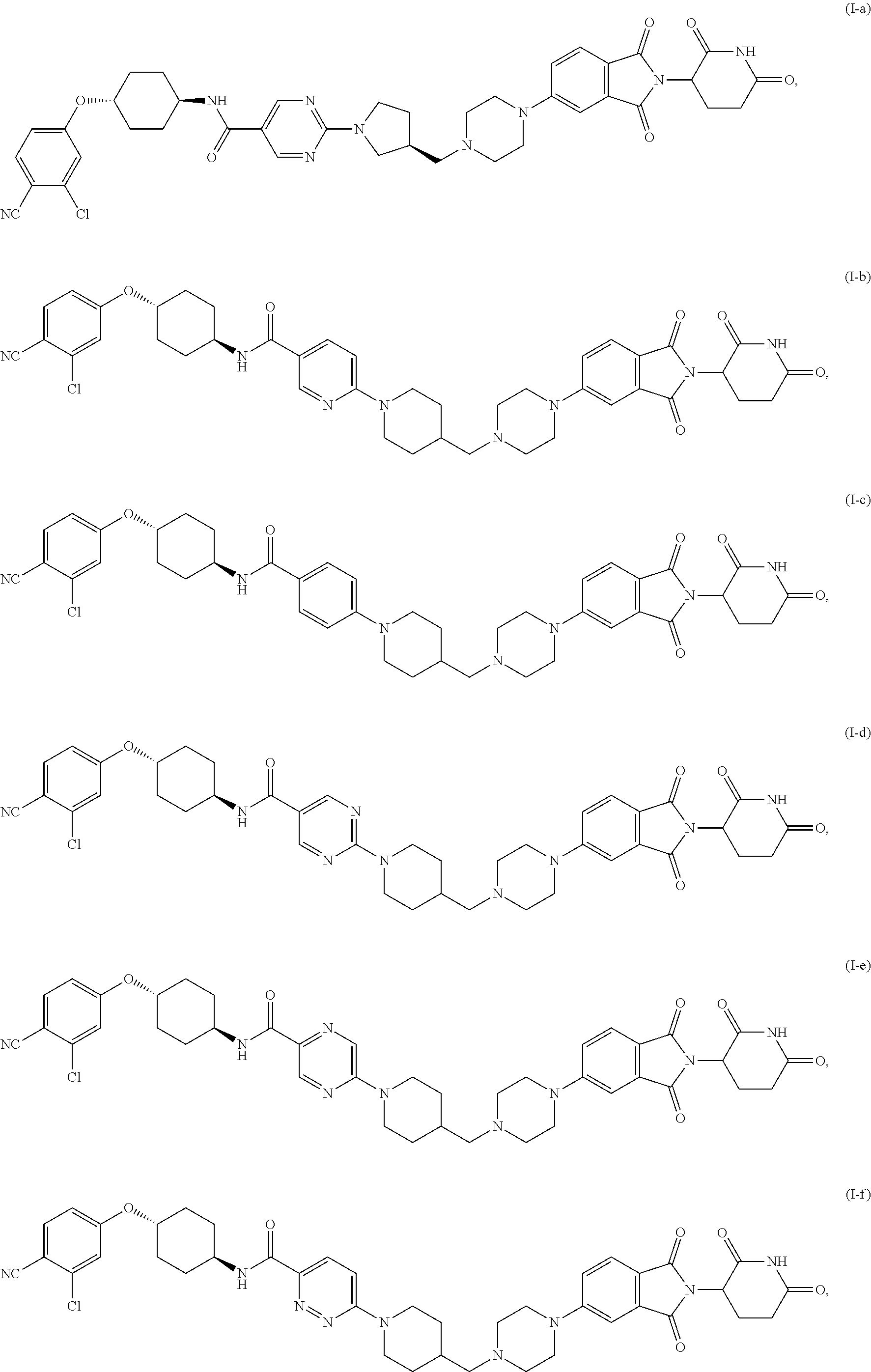

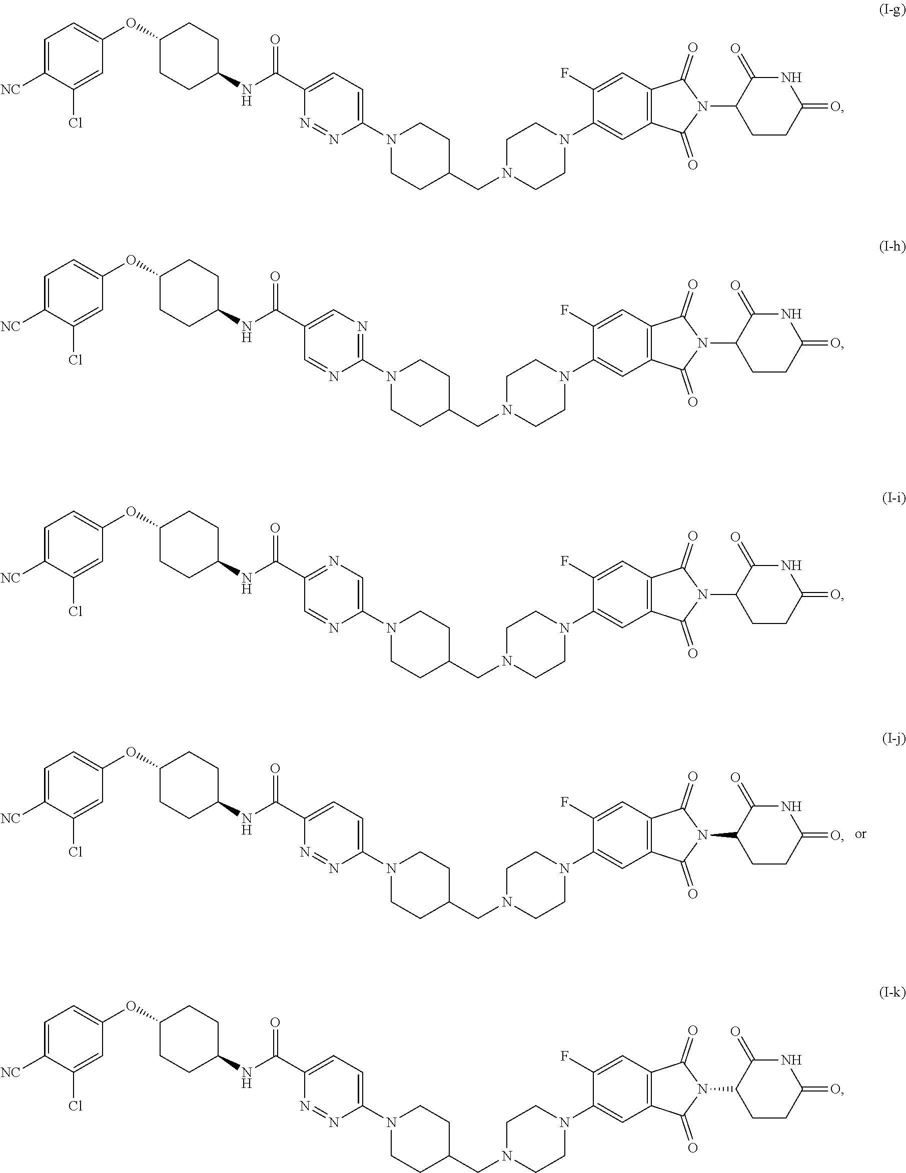

or a pharmaceutically acceptable salt, enantiomer, stereoisomer, solvate, polymorph, isotopic derivative, or prodrug thereof.

In some embodiments, the compound of Formula (I) is

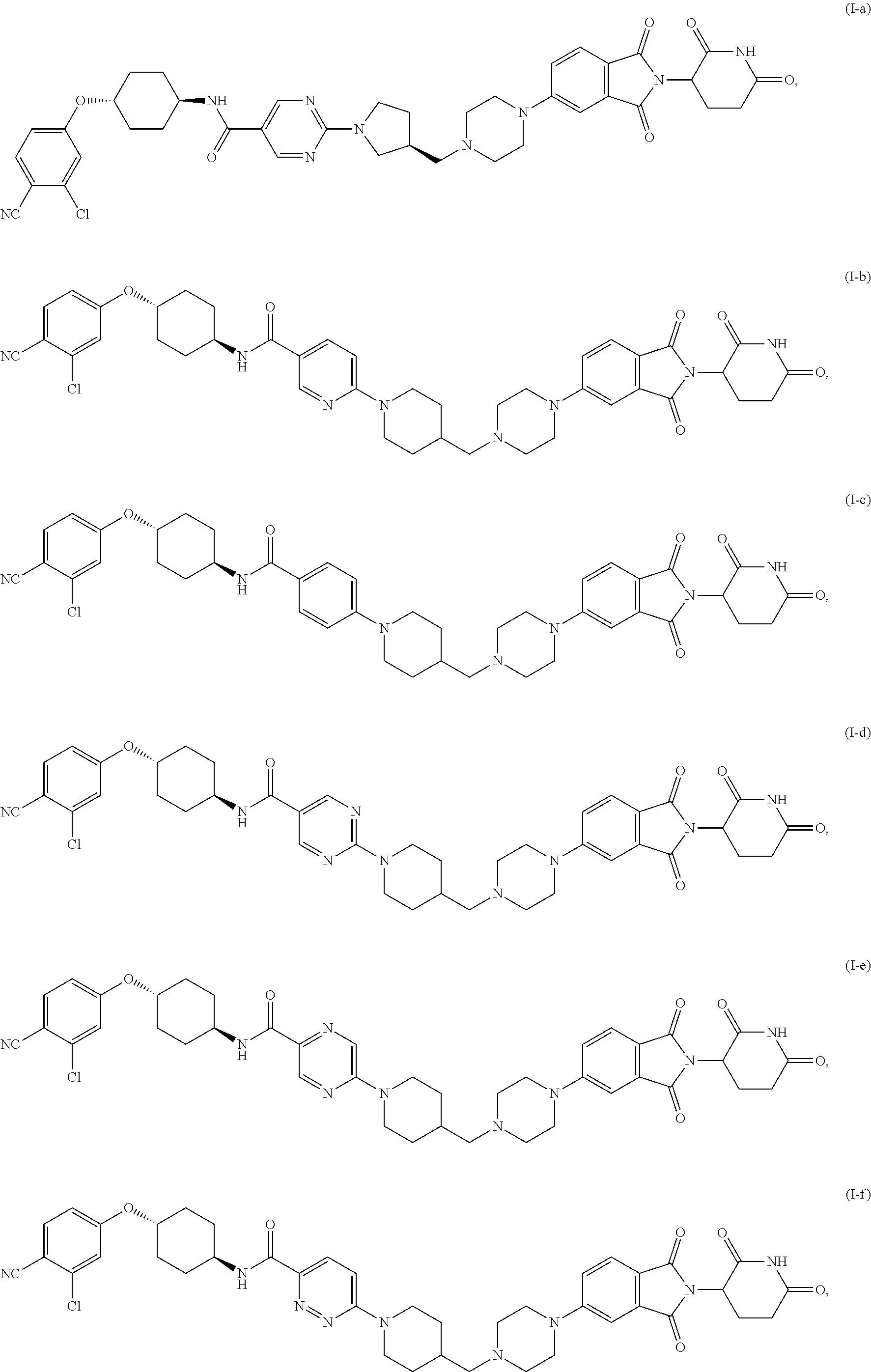

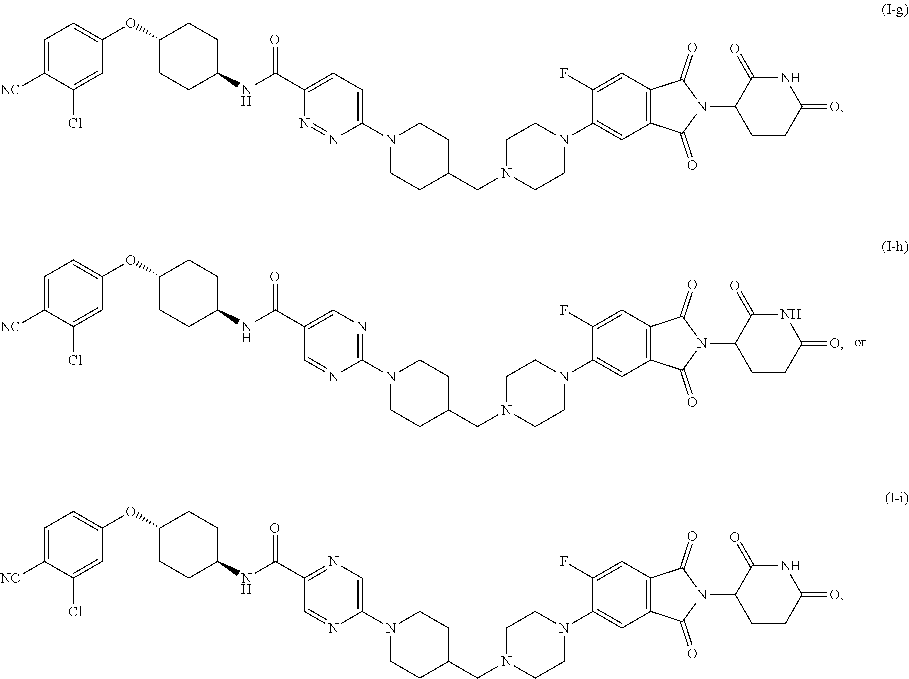 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In some embodiments, the compound of Formula (I) is

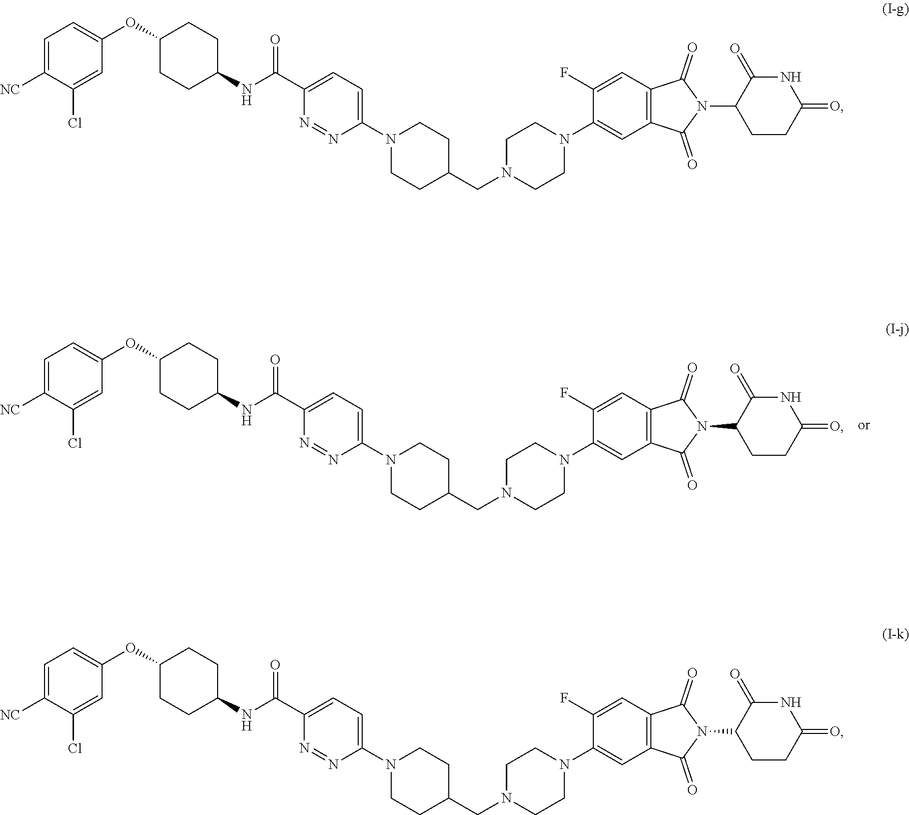

or a pharmaceutically acceptable salt, solvate, polymorph, isotopic derivative, or prodrug thereof.

In some embodiments, the compound of Formula (I) is the compound of Formula (I-a):

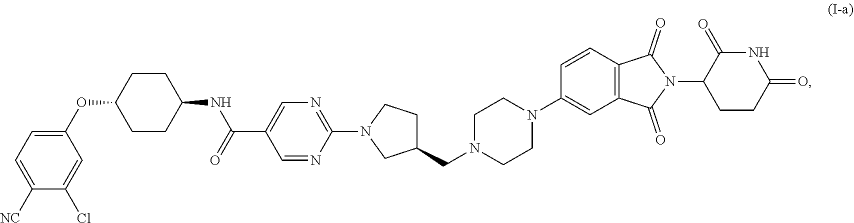 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-b):

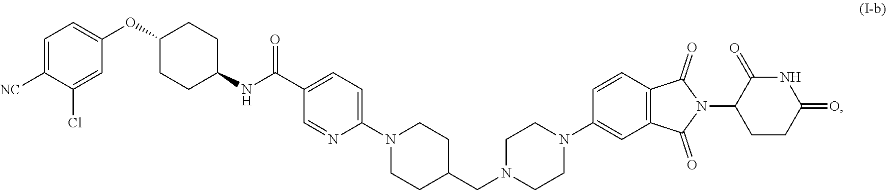 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-c):

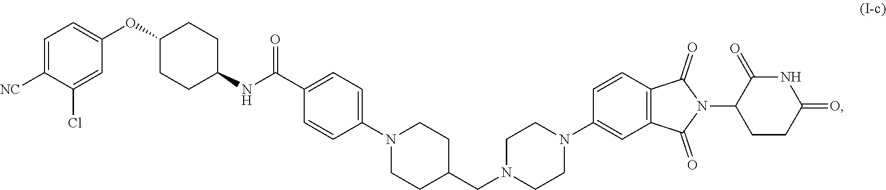 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-d):

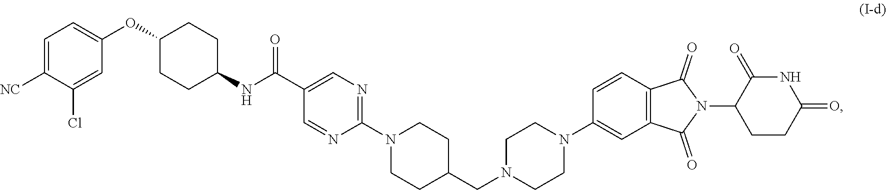 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-e):

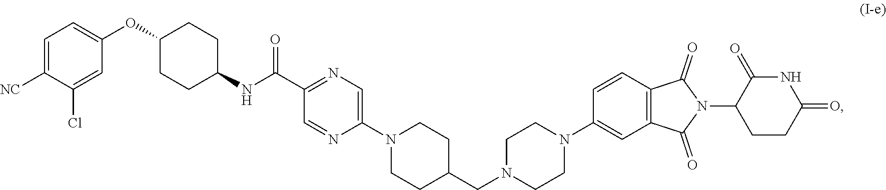 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-f):

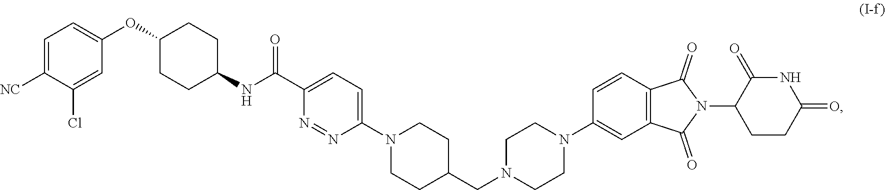 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nn3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-g):

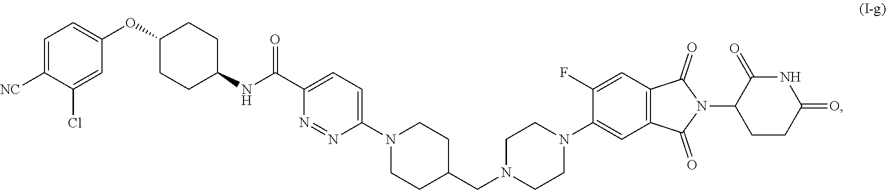 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-h):

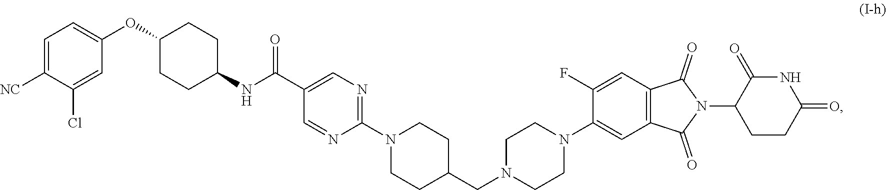 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-i):

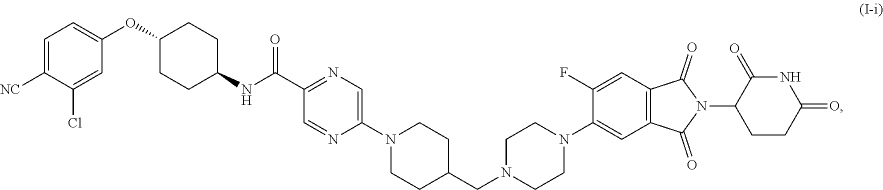 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

or a pharmaceutically acceptable salt, enantiomer, stereoisomer, solvate, polymorph, isotopic derivative, or prodrug thereof.

In some embodiments, the compound of Formula (I) is the compound of Formula (I-j):

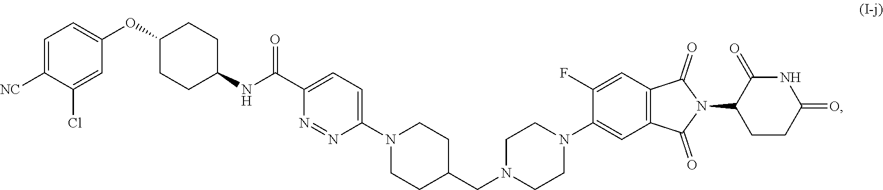

or a pharmaceutically acceptable salt, enantiomer, stereoisomer, solvate, polymorph, isotopic derivative, or prodrug thereof.

In some embodiments, the compound of Formula (I) is the compound of Formula (I-k):

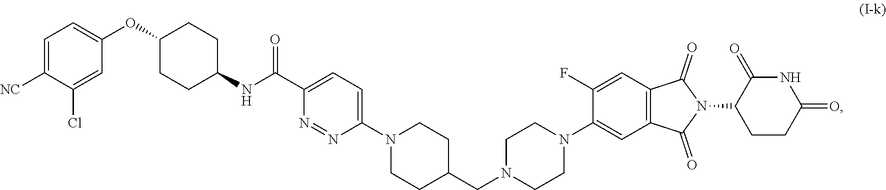

or a pharmaceutically acceptable salt, enantiomer, stereoisomer, solvate, polymorph, isotopic derivative, or prodrug thereof.

In some embodiments, the compound of Formula (I) is

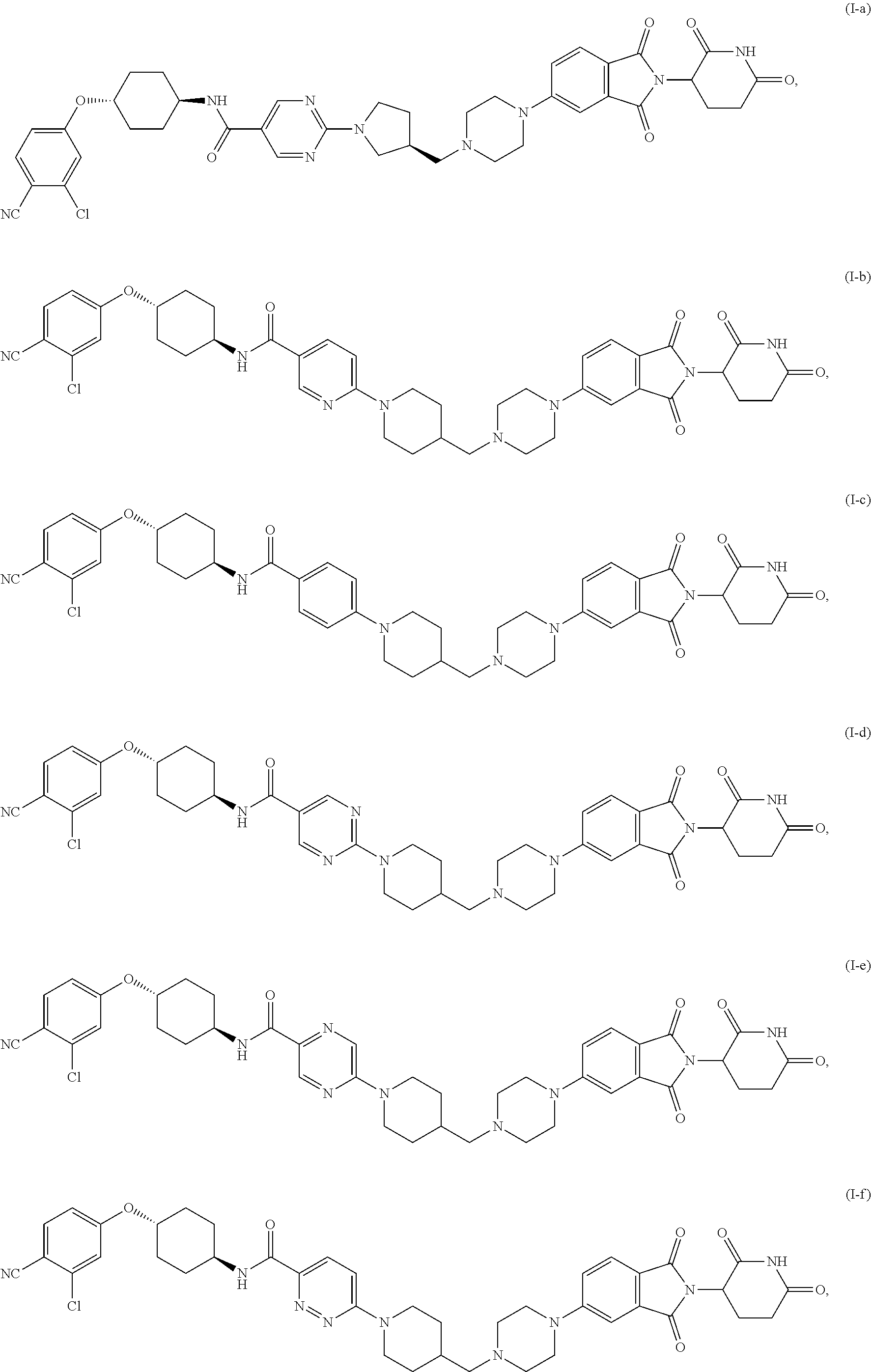

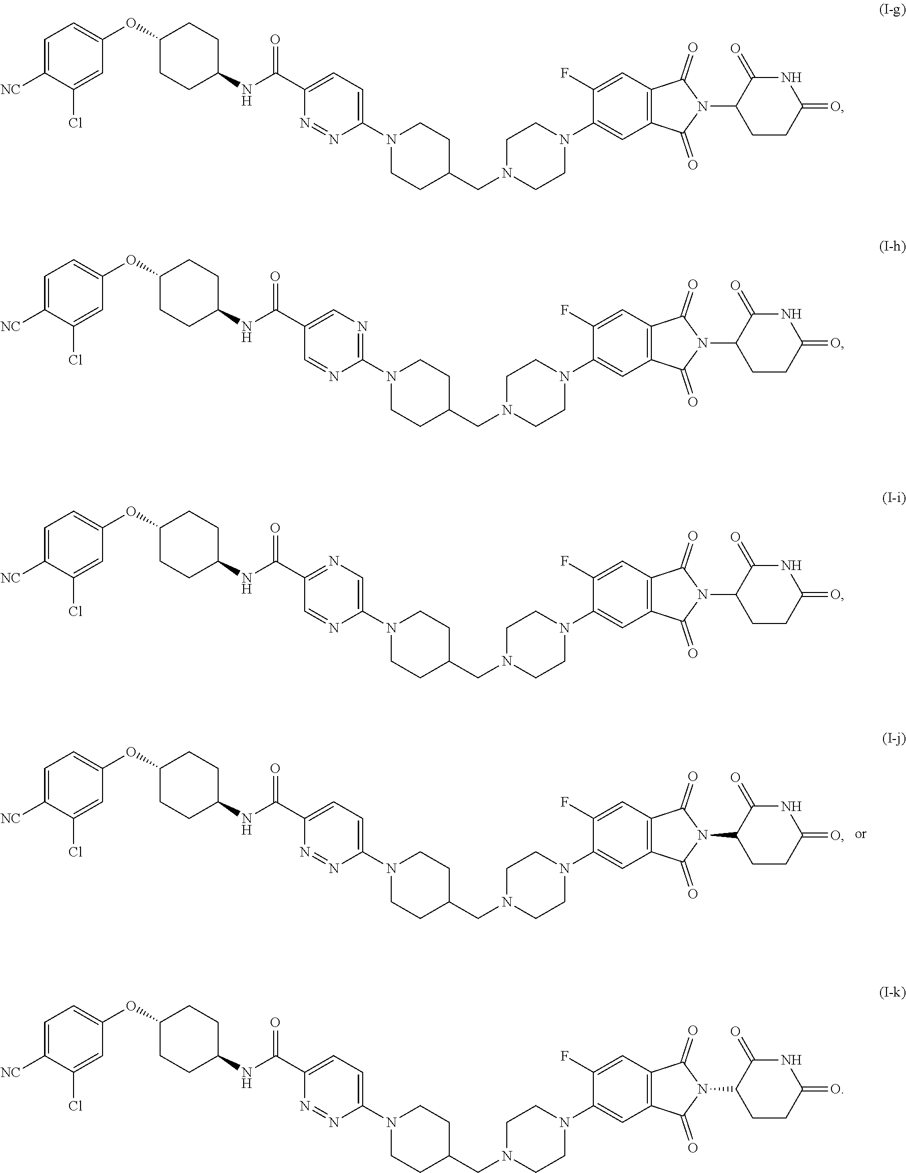

In some embodiments, the compound of Formula (I) is

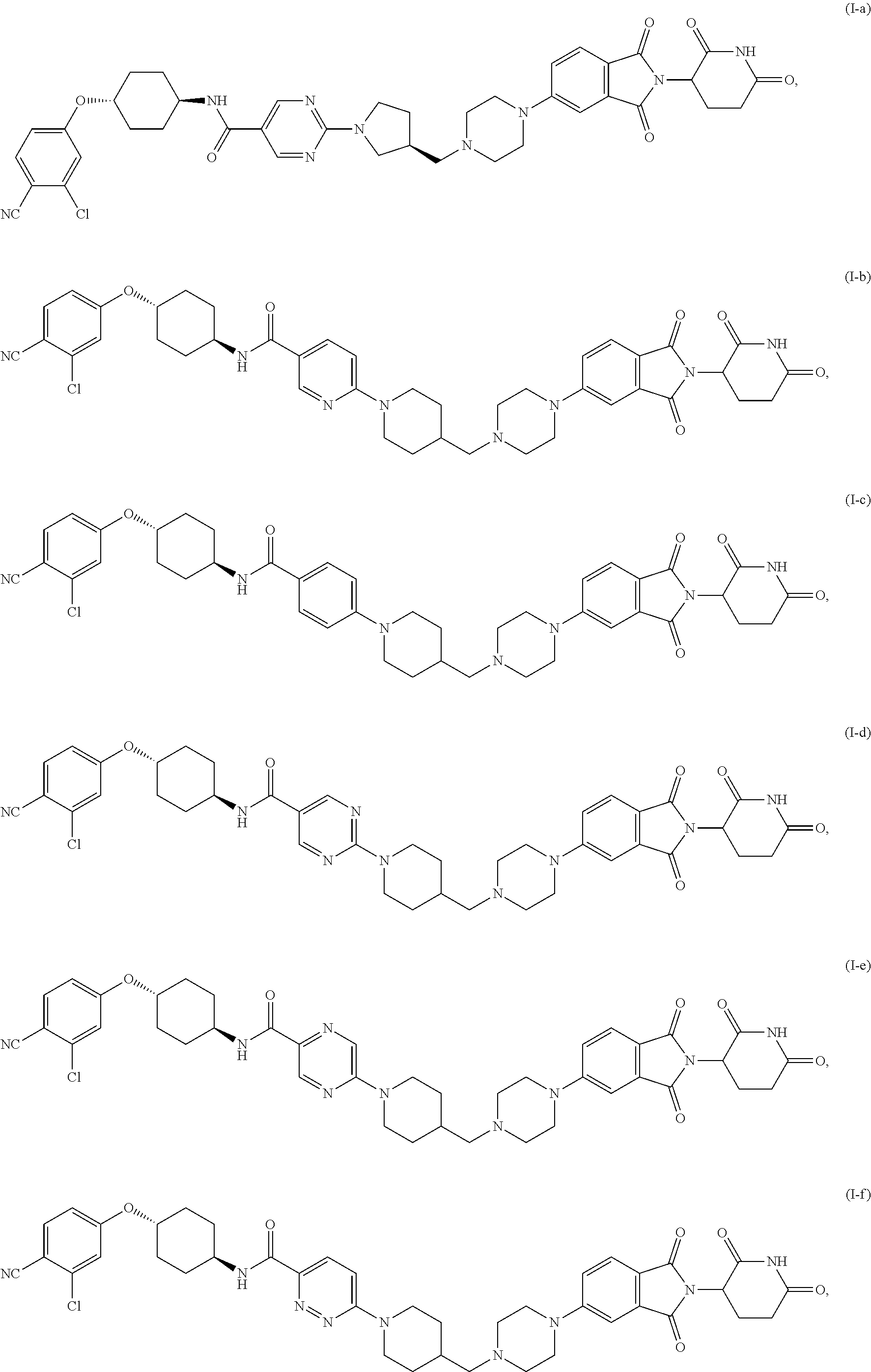

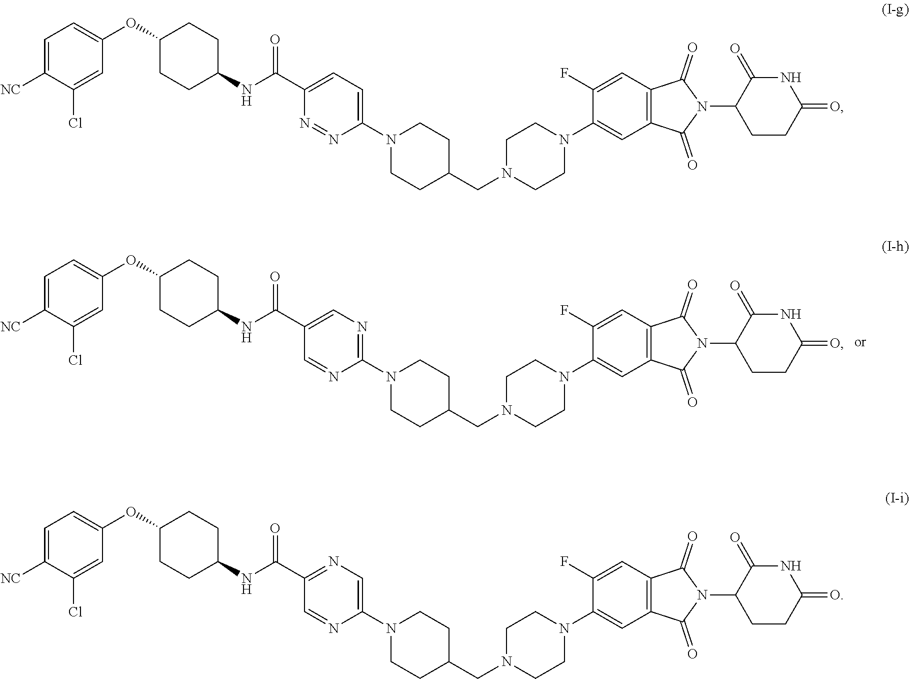 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl ; "or" ; N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

In some embodiments, the compound of Formula (I) is

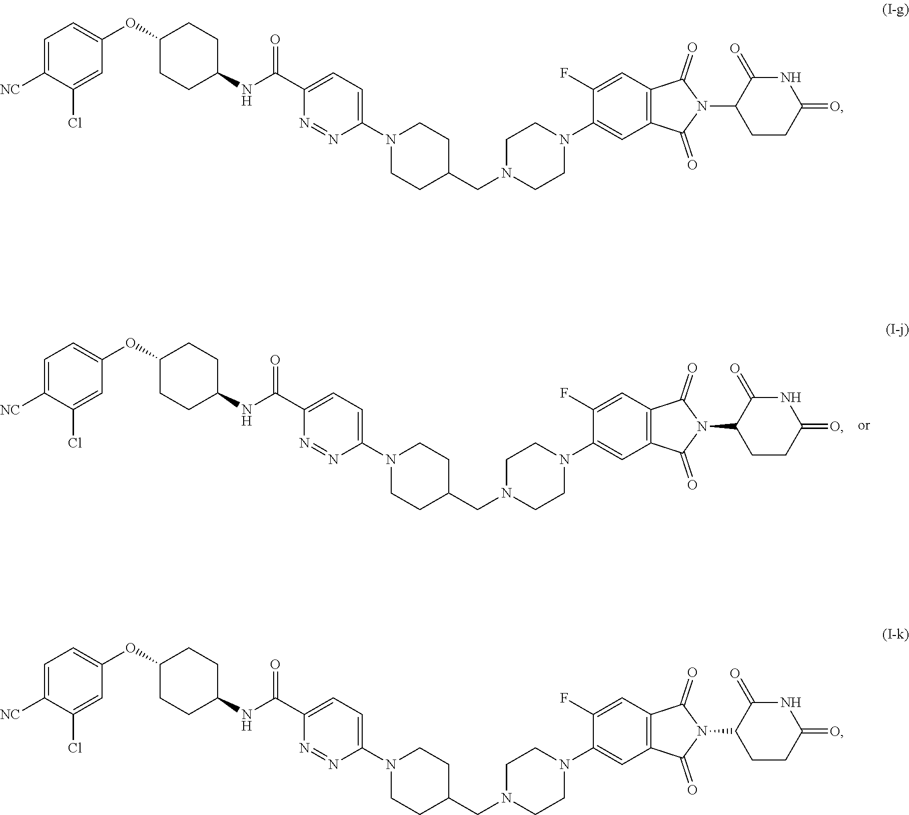

In some embodiments, the compound of Formula (I) is the compound of Formula (I-a):

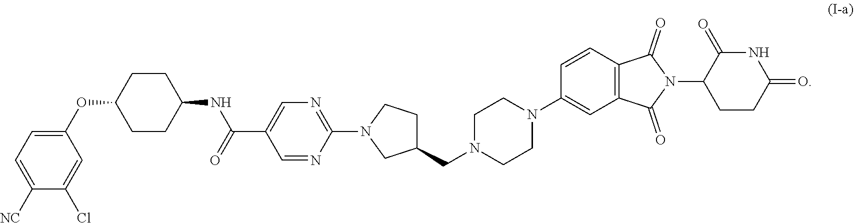 [SMILES: N#Cc1ccc(O[C@H]2CC[C@H](NC(=O)c3cnc(N4CC[C@@H](CN5CCN(c6ccc7c(c6)C(=O)N(C6CCC(=O)NC6=O)C7=O)CC5)C4)nc3)CC2)cc1Cl]

In some embodiments, the compound of Formula (I) is the compound of Formula (I-b):

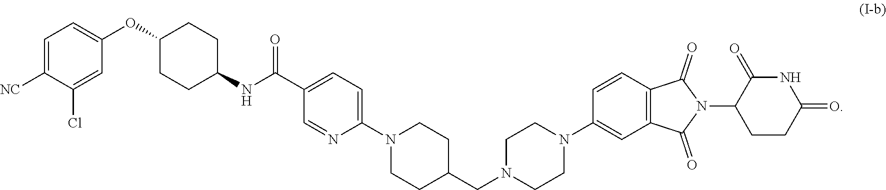 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nc3)CC2)cc1Cl]

In some embodiments, the compound of Formula (I) is the compound of Formula (I-c):

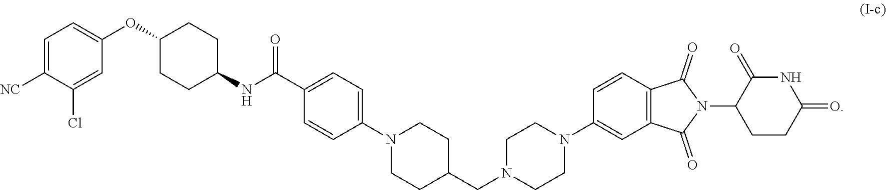 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)cc3)CC2)cc1Cl]

In some embodiments, the compound of Formula (I) is the compound of Formula (I-d):

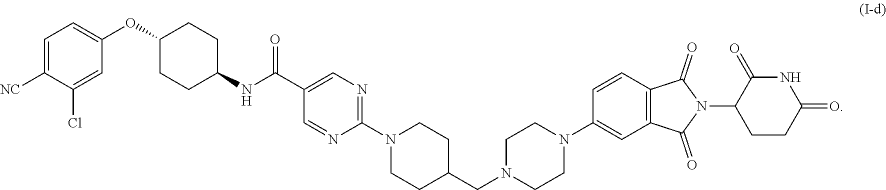 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)nc3)CC2)cc1Cl]

In some embodiments, the compound of Formula (I) is the compound of Formula (I-e):

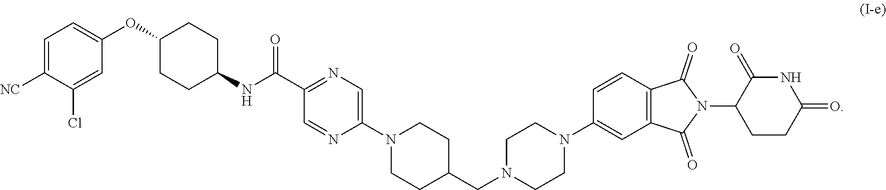 [SMILES: N#Cc1ccc(O[C@H]2CC[C@H](NC(=O)c3cnc(N4CCC(CN5CCN(c6ccc7c(c6)C(=O)N(C6CCC(=O)NC6=O)C7=O)CC5)CC4)cn3)CC2)cc1Cl]

In some embodiments, the compound of Formula (I) is the compound of Formula (I-f):

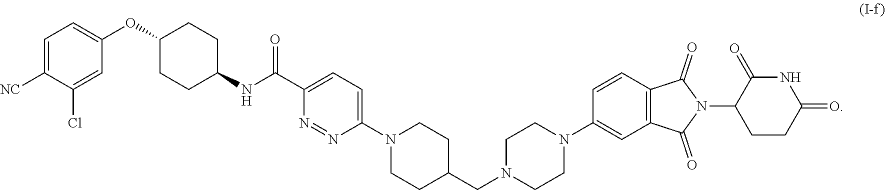 [SMILES: N#Cc1ccc(O[C@H]2CC[C@H](NC(=O)c3ccc(N4CCC(CN5CCN(c6ccc7c(c6)C(=O)N(C6CCC(=O)NC6=O)C7=O)CC5)CC4)nn3)CC2)cc1Cl]

In some embodiments, the compound of Formula (I) is the compound of Formula (I-g):

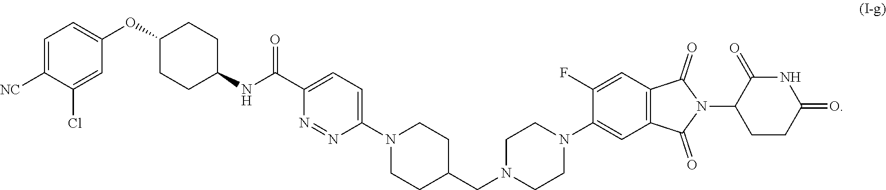 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

In some embodiments, the compound of Formula (I) is the compound of Formula (I-h):

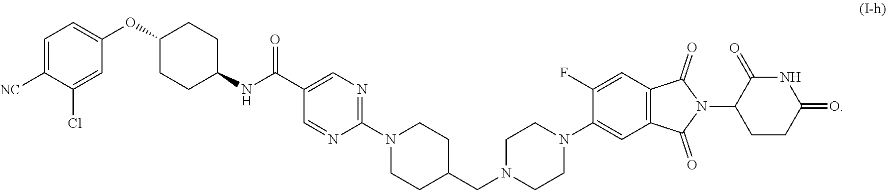 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)nc3)CC2)cc1Cl]

In some embodiments, the compound of Formula (I) is the compound of Formula (I-j):

In some embodiments, the compound of Formula (I) is the compound of Formula (I-i):

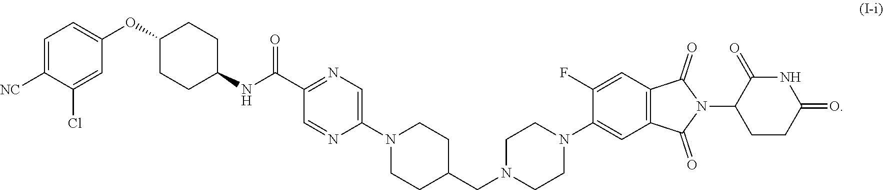 [SMILES: N#Cc1ccc(O[C@H]2CC[C@H](NC(=O)c3cnc(N4CCC(CN5CCN(c6cc7c(cc6F)C(=O)N(C6CCC(=O)NC6=O)C7=O)CC5)CC4)cn3)CC2)cc1Cl]

In some embodiments, the compound of Formula (I) is the compound of Formula (I-j):

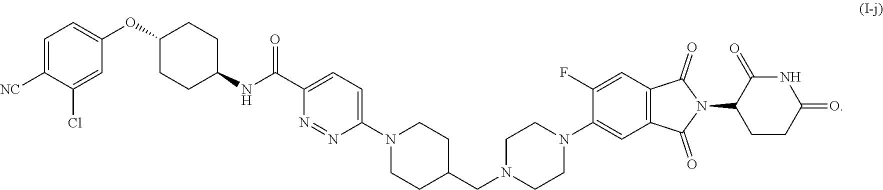

In some embodiments, the compound of Formula (I) is the compound of Formula (I-k):

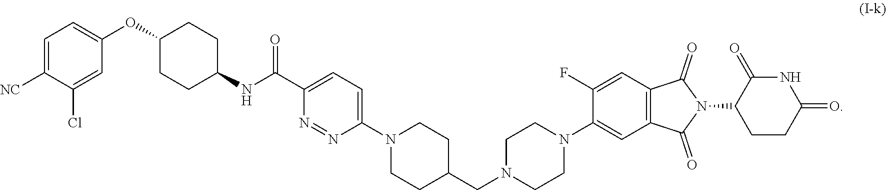

A compound of Formula (I) may be synthesized using standard synthetic methods and procedures for the preparation of organic molecules and functional group transformations and manipulations, including the use of protective groups, as can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Wuts, P. G. M. Protective Groups in Organic Synthesis, 3^{rd}; John Wiley & Sons: New York, 1999. A method for preparing a compound of Formula (I) is described in U.S. Patent Application Publication No. 2018/0099940, now U.S. Pat. No. 10,584,101, the contents of which are incorporated herein in their entirety.

### Methods of Ubiquitinating/Degrading a Target Protein in a Cell

The present invention provides a method of ubiquitinating/degrading a target protein in a cell. The method comprises administering a bifunctional composition comprising an E3 ubiquitin ligase binding moiety and a protein targeting moiety, preferably linked through a linker moiety, as otherwise described herein, wherein the E3 ubiquitin ligase binding moiety is coupled to the protein targeting moiety and wherein the E3 ubiquitin ligase binding moiety recognizes a ubiquitin pathway protein (e.g., an ubiquitin ligase, preferably an E3 ubiquitin ligase) and the protein targeting moiety recognizes the target protein such that degradation of the target protein will occur when the target protein is placed in proximity to the ubiquitin ligase, thus resulting in degradation/inhibition of the effects of the target protein and the control of protein levels. The control of protein levels afforded by the present invention provides treatment of a disease state or condition, which is modulated through the target protein by lowering the level of that protein in the cells of a patient.

In one aspect, this application provides a compound of Formula (I), or a pharmaceutically acceptable salt, enantiomer, stereoisomer, solvate, polymorph, isotopic derivative, or prodrug thereof, that degrades the androgen receptor (AR) protein. In some embodiments, the AR that is degraded by the compound of Formula (I) is wild type AR. In some embodiments, the AR that is degraded by the compound of Formula (I) is a mutant form of AR.

As understood by the skilled artisan, AR has a modular structure comprising three functional domains: the N-terminal transcriptional regulation domain, the DNA-binding domain, and the ligand binding domain (MacLean H E, et al J. Steroid Biochem Mol Biol. (1997) 62:233-42). The DNA-binding domain is linked to the ligand-binding domain via a hinge. The AR ligand binding domain refers to the functional domain of human AR that folds to form a hydrophobic pocket that binds to the AR cognate hormone ligand (e.g., androgen).

Moreover, it is understood in the art that AR is 920 amino acid residues in length, wherein the N-terminal transcriptional regulation domain extends from amino acid residue 1 to about amino acid residue 559, the DNA-binding domain extends from about amino acid residue 560 to about amino acid residue 624, the hinge extends from about amino acid residue 625 to about amino acid residue 676, and the ligand binding domain extends from about amino acid residue 677 to about amino acid residue 920. A suitable AR reference sequence is set forth by SEQ ID NO: 1 and identified in the UniProt database as P10275 (ANDR_HUMAN). The gene encoding AR (“the AR gene”) is approximately 90 kb and has chromosomal coordinates 67544021-67730619 according to human reference genome GRCh38.p13. The AR gene contains 8 exons, with exon 1 encoding the N-terminal transcriptional regulation domain; exon 2-3 encoding the DNA-binding domain; and exons 4-8 encoding the hinge and ligand binding domain (Jenster, et al (1992) J. Steroid Biochem. Mol. Biol. 41:671-75).

In some embodiments, the subject has a prostate cancer comprising at least one somatic AR tumor mutation in a functional domain of AR. In some embodiments, the at least one somatic AR tumor mutation is an insertion, deletion, or substitution of one or more amino acid residues in an AR functional domain as compared to an AR reference sequence (e.g., SEQ ID NO: 1). In some embodiments, the at least one somatic AR tumor mutation is a substitution of one or more amino acid residues in an AR functional domain as compared to an AR reference sequence (e.g., SEQ ID NO: 1). In some embodiments, the at least one somatic AR tumor mutation is an insertion, deletion, or substitution of one or more amino acid residues in the AR ligand binding domain as compared to an AR reference sequence (e.g., SEQ ID NO: 1). In some embodiments, the at least one somatic AR tumor mutation is a substitution of one or more amino acid residues in the AR ligand binding domain as compared to an AR reference sequence (e.g., SEQ ID NO: 1). In some embodiments, the at least one somatic AR tumor mutation is an insertion, deletion or substitution of one or more amino acid residues selected from amino acid residues 677-920 as compared to an AR reference sequence, wherein the AR reference sequence is set forth by SEQ ID NO: 1. In some embodiments, the at least one somatic AR tumor mutation is a substitution of one or more amino acid residues selected from amino acid residues 677-920 as compared to an AR reference sequence, wherein the AR reference sequence is set forth by SEQ ID NO: 1.

In some embodiments, the alteration (e.g., substitution) of an amino acid residue in the AR ligand binding domain provides a mutant AR having reduced ligand specificity and/or enhanced cofactor recruitment. Without being bound by theory, a mutant AR having a reduced ligand specificity and/or enhanced cofactor recruitment has increased potency for triggering the AR signaling pathway, thereby conferring a growth advantage on a tumor cell comprising the mutant AR.

In some embodiments, the prostate cancer comprises cancer cells characterized by expression of at least one somatic AR tumor mutation described herein. Methods to identify a cancer characterized by expression of somatic mutations are known in the art, and include, e.g., obtaining a biological sample from the subject, harvesting the biological sample to obtain genetic material (e.g., genomic DNA or RNA), and performing sequencing analysis, RNA-sequencing analysis, or real-time polymerase chain reaction (RT-PCR). For example, in some embodiments, genomic DNA is first obtained (using any standard technique) from cancerous tissue obtained from the subject, cDNA is prepared, and amplification is performed (e.g., using a polymerase chain reaction) to provide the cDNA in sufficient quantity for sequence analysis, and sequencing is performed using, e.g., next generation sequencing. Genomic DNA or RNA is typically extracted from biological samples such as tissues removed from the subject, e.g., by tissue biopsy. In some embodiments, the biological sample is a tissue biopsy sample (e.g., a prostate biopsy sample), wherein sequence analysis of genomic DNA or RNA is performed to identify the presence of somatic mutations in AR (e.g., a somatic mutation resulting in a substitution of an amino acid residue in the AR ligand binding domain). In some embodiments, the biological sample comprises plasma obtained from the subject is used to detect somatic AR tumor mutations present in circulating tumor DNA, e.g., using PCR-based amplification, followed by gene sequencing.

In some embodiments, the mutant form of AR that is degraded by the compound of Formula (I) comprises at least one AR somatic tumor mutation.

In some embodiments, the at least one somatic AR tumor mutation is selected from any one or any combination of L702X, T878X, H875X, W742X, F877X, V716X, D891X, M750X, and S889X, wherein “X” refers to any amino acid residue other than the wild type residue at that position. In some embodiments, the at least one somatic AR tumor mutation is selected from any one or any combination of L702X, T878X, H875X, W742X, F877X, V716X, D891X, M750X, and S889X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from any one or any combination of H875X, Q825X, T878X, F877X, V716X, T878X, W742X, D891X, M750X, and S889X, wherein “X” refers to any amino acid residue other than the wild type residue at that position. In some embodiments, the at least one somatic AR tumor mutation is selected from any one or any combination of H875X, Q825X, T878X, F877X, V716X, T878X, W742X, D891X, M750X, and S889X wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least one somatic AR tumor mutation is selected from any one or any combination of L702H, T878A, H875Y, W742C, W742L, F877L, T878S, V716M, D891H, M750V, M750T, and S889G. In some embodiments, the at least one somatic AR tumor mutation is selected from any one or any combination of E666K, Q799E, Q793E, Q118K, Y447N, S532Y, G751C, Q825E, L702H, T878A, H875Y, H875L, Q825E, W742C, W742L, F877L, T878S, V716M, D891H, M750V, M750T, and S889G. In some embodiments, the at least one somatic AR tumor mutation is selected from any one or any combination of E666K, Q799E, Q793E, Q118K, Y447N, S532Y, G751C, Q825E, T878A, H875Y, H875L, Q825E, W742C, W742L, F877L, T878S, V716M, D891H, M750V, M750T, and S889G.

In some embodiments, the at least one somatic AR tumor mutation is selected from any one or any combination of T878A, H875Y, H875L, Q825E, W742C, W742L, F877L, T878S, V716M, D891H, M750V, M750T, and S889G. In some embodiments, the at least one somatic AR tumor mutation is selected from any one or any combination of H875Y, H875L, T878A, F877L, V716M, T878S, W742C, and W742L. In some embodiments, the at least one somatic AR tumor mutation is selected from any one or any combination of H875Y, H875L, Q825E, T878A, F877L, V716M, T878S, W742C, and W742L. In some embodiments, the at least one somatic AR tumor mutation is L702H. In some embodiments, the at least one somatic AR tumor mutation is T878A. In some embodiments, the at least one somatic AR tumor mutation is H875Y. In some embodiments, the at least one somatic AR tumor mutation is H875L. In some embodiments, the at least one somatic AR tumor mutation is Q825E. In some embodiments, the at least one somatic AR tumor mutation is W742C. In some embodiments, the at least one somatic AR tumor mutation is W742L. In some embodiments, the at least one somatic AR tumor mutation is F877L. In some embodiments, the at least one somatic AR tumor mutation is T878S. In some embodiments, the at least one somatic AR tumor mutation is V716M. In some embodiments, the at least one somatic AR tumor mutation is D891H. In some embodiments, the at least one somatic AR tumor mutation is M750V. In some embodiments, the at least one somatic AR tumor mutation is M750T. In some embodiments, the at least one somatic AR tumor mutation is S889G.

In some embodiments, the at least one somatic AR tumor mutation is selected from L702H, T878A, H875Y, W742C, W742L, F877L, T878S, V716M, D891H, M750V, M750T, and S889G.

In some embodiments, the mutant form of AR that is degraded by the compound of Formula (I) comprises at least two AR somatic tumor mutations.

In some embodiments, the at least two somatic AR tumor mutations are selected from any one or any combination of L702X, T878X, H875X, W742X, F877X, V716X, D891X, M750X, and S889X, wherein “X” refers to any amino acid residue other than the wild type residue at that position. In some embodiments, the at least two somatic AR tumor mutations are selected from any one or any combination of L702X, T878X, H875X, W742X, F877X, V716X, D891X, M750X, and S889X, wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least two somatic AR tumor mutations are selected from any one or any combination of H875X, Q825X, T878X, F877X, V716X, T878X, W742X, D891X, M750X, and S889X, wherein “X” refers to any amino acid residue, other than the wild type residue at that position. In some embodiments, the at least two somatic AR tumor mutations are selected from any one or any combination of H875X, Q825X, T878X, F877X, V716X, T878X, W742X, D891X, M750X, and S889X wherein “X” refers to an amino acid residue, other than the wild type residue at that position, selected from alanine (A); valine (V); leucine (L); isoleucine (I); phenylalanine (F); methionine (M); tryptophan (W); proline (P); glycine (G); serine (S); threonine (T); cysteine (C); asparagine (N); glutamine (Q); tyrosine (Y); lysine (K); arginine (R); histidine (H); aspartate (D); and glutamate (E).

In some embodiments, the at least two somatic AR tumor mutations are selected from any one or any combination of L702H, T878A, H875Y, W742C, W742L, F877L, T878S, V716M, D891H, M750V, M750T, and S889G. In some embodiments, the at least two somatic AR tumor mutations are selected from any one or any combination of E666K, Q799E, Q793E, Q118K, Y447N, S532Y, G751C, Q825E, L702H, T878A, H875Y, H875L, Q825E, W742C, W742L, F877L, T878S, V716M, D891H, M750V, M750T, and S889G. In some embodiments, the at least two somatic AR tumor mutations are selected from any one or any combination of E666K, Q799E, Q793E, Q118K, Y447N, S532Y, G751C, Q825E, T878A, H875Y, H875L, Q825E, W742C, W742L, F877L, T878S, V716M, D891H, M750V, M750T, and S889G.

In some embodiments, the at least two somatic AR tumor mutations are selected from any one or any combination of T878A, H875Y, H875L, Q825E, W742C, W742L, F877L, T878S, V716M, D891H, M750V, M750T, and S889G. In some embodiments, the at least two somatic AR tumor mutations are selected from any one or any combination of H875Y, H875L, T878A, F877L, V716M, T878S, W742C, and W742L. In some embodiments, the at least two somatic AR tumor mutations are selected from any one or any combination of H875Y, H875L, Q825E, T878A, F877L, V716M, T878S, W742C, and W742L.

In some embodiments, the at least two somatic AR tumor mutation are selected from L702H, H875Y, T878A, F877L, V716M, T878S, W742C, and W742L. In some embodiments, the at least two somatic AR tumor mutations are selected from the following groups of mutations:

L702H and H875Y;

L702H, T878A, and H875Y;

T878A, F877L, L702H, and V716M;

T878S and H875Y;

T878S and W742C;

W742C and W742L; and

L702H and T878A.

In some embodiments, the at least two somatic AR tumor mutations are selected from the following groups of mutations:

L702H and H875Y;

L702H, T878A, and H875Y;

H875L and Q825E;

T878A, F877L, and V716M;

T878A, L702H, M750T, and D891H;

T878S and H875Y;

T878A and T878S;

T878S and W742C;

W742C and W742L; and

L702H and T878A.

In some embodiments, the at least two somatic AR tumor mutations are selected from the following groups of mutations:

T878A, and H875Y;

H875L and Q825E;

T878A, F877L, and V716M;

T878A, M750T, and D891H;

T878S and H875Y;

T878A and T878S;

T878S and W742C;

W742C and W742L.

In some embodiments, the present disclosure is directed to a method of treating a patient in need for a disease state or condition modulated through a protein where the degradation of that protein will produce a therapeutic effect in that patient, the method comprising administering to a patient in need an effective amount of a compound of Formula (I), optionally in combination with another bioactive agent (e.g., an anti-cancer agent). The disease state or condition may be a disease caused by a microbial agent or other exogenous agent such as a virus, bacteria, fungus, protozoa or other microbe or may be a disease state, which is caused by overexpression of a protein, which leads to a disease state and/or condition.

### Methods of Treatment

In one aspect, the present application pertains to a method of treating and/or preventing cancer comprising administering to a subject in need thereof a therapeutically effective amount of a compound of Formula (I), or a pharmaceutically acceptable salt, enantiomer, stereoisomer, solvate, polymorph, isotopic derivative, or prodrug thereof.

In one aspect, the present application pertains to a method of treating and/or preventing cancer comprising administering to a subject in need thereof a therapeutically effective amount of a compound of Formula (I), or a pharmaceutically acceptable salt, enantiomer, stereoisomer, solvate, polymorph, isotopic derivative, or prodrug thereof, in combination with one or more additional anti-cancer agents.

In one aspect, the present application pertains to a method of treating prostate cancer in a subject in need thereof, wherein the subject with prostate cancer comprises at least one somatic AR tumor mutation;
the method comprising administering to the subject a therapeutically effective amount of a compound of Formula (I),

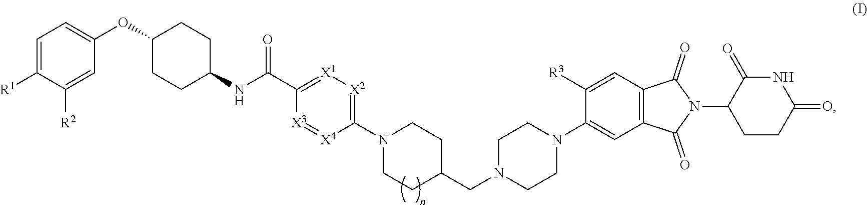

or a
pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH. In some embodiments, the prostate cancer comprises at least one somatic AR tumor mutation.

In one aspect, the present application pertains to a method of treating prostate cancer in a subject in need thereof, wherein the prostate cancer comprises at least one somatic AR tumor mutation;
the method comprising administering to the subject a therapeutically effective amount of a compound of Formula (I),

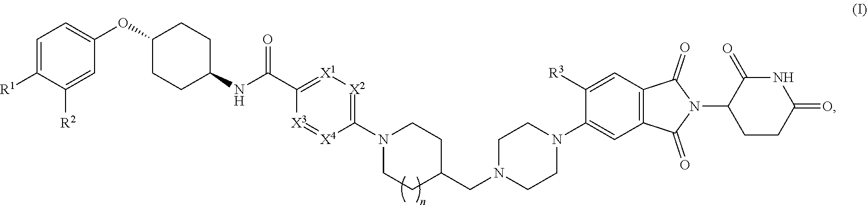

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH.

In one aspect, the present application pertains to a method of treating prostate cancer in a subject in need thereof, wherein the subject with prostate cancer comprises at least one somatic AR tumor mutation;
the method comprising administering to the subject a therapeutically effective amount of a compound of Formula (I),

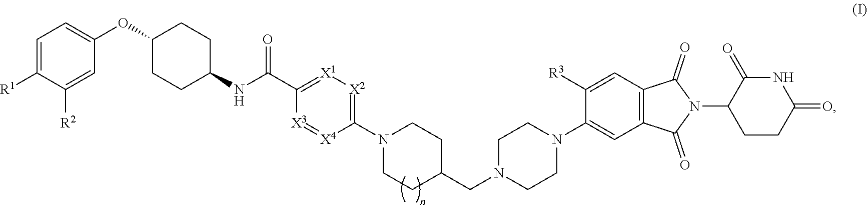

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and wherein the therapeutically effective amount of the compound of Formula (I) is about 35 mg to about 1000 mg. In some embodiments, the prostate cancer comprises at least one somatic AR tumor mutation.

In one aspect, the present application pertains to a method of treating prostate cancer in a subject in need thereof, wherein the prostate cancer comprises at least one somatic AR tumor mutation;
the method comprising administering to the subject a therapeutically effective amount of a compound of Formula (I),

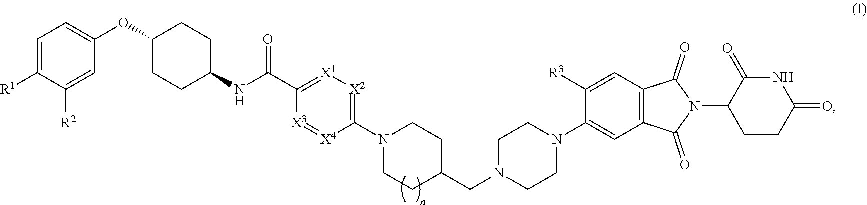

or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and wherein the therapeutically effective amount of the compound of Formula (I) is about 35 mg to about 1000 mg.

The methods of treating cancer described herein include a reduction in tumor size. Alternatively, or in addition, the cancer is metastatic cancer and this method of treatment includes inhibition of metastatic cancer cell invasion.

In some embodiments, the cancer is prostate cancer. In some embodiments, the cancer is adenocarcinoma of the prostate.

In some embodiments, the cancer is metastatic prostate cancer.

In some embodiments, the cancer is castrate-resistant prostate cancer.

In some embodiments, the cancer is metastatic, castrate-resistant prostate cancer (mCRPC).

In some embodiments, the subject suffering from prostate cancer (e.g., mCRPC) will have a different response to treatment with a compound of Formula (I), or a pharmaceutically acceptable salt, enantiomer, stereoisomer, solvate, polymorph, isotopic derivative, or prodrug thereof, depending on the AR biomarker status of the subject, i.e., whether the subject has one or more somatic tumor mutations to AR.

In some embodiments, the subject with prostate cancer comprises at least one somatic AR tumor mutation. In some embodiments, the prostate cancer comprises at least one somatic AR tumor mutation.

In some embodiments, the prostate cancer comprises at least the somatic AR tumor mutation of L702H. In some embodiments, the prostate cancer comprises at least the somatic AR tumor mutation of T878A. In some embodiments, the prostate cancer comprises at least the somatic AR tumor mutation of H875Y. In some embodiments, the prostate cancer comprises at least the somatic AR tumor mutation of H875L. In some embodiments, the prostate cancer comprises at least the somatic AR tumor mutation of W742C. In some embodiments, the prostate cancer comprises at least the somatic AR tumor mutation of Q825E. In some embodiments, the prostate cancer comprises at least the somatic AR tumor mutation of W742L. In some embodiments, the prostate cancer comprises at least the somatic AR tumor mutation of F877L. In some embodiments, the prostate cancer comprises at least the somatic AR tumor mutation of T878S. In some embodiments, the prostate cancer comprises at least the somatic AR tumor mutation of V716M. In some embodiments, the prostate cancer comprises at least the somatic AR tumor mutation of D891H. In some embodiments, the prostate cancer comprises at least the somatic AR tumor mutation of M750V. In some embodiments, the prostate cancer comprises at least the somatic AR tumor mutation of M750T. In some embodiments, the prostate cancer comprises at least the somatic AR tumor mutation of S889G.

In some embodiments, the subject with prostate cancer comprises at least two somatic AR tumor mutations. In some embodiments, the prostate cancer comprises at least two somatic AR tumor mutations. In some embodiments, the prostate cancer comprises at least the two somatic AR tumor mutations L702H and H875Y. In some embodiments, the prostate cancer comprises at least the two somatic AR tumor mutations of T878A, and H875Y. In some embodiments, the prostate cancer comprises at least the two somatic AR tumor mutations of H875L and Q825E. In some embodiments, the prostate cancer comprises at least the three somatic AR tumor mutations of L702H, T878A, and H875Y. In some embodiments, the prostate cancer comprises at least the four somatic AR tumor mutations of T878A, F877L, L702H, and V716M. In some embodiments, the prostate cancer comprises at least the three somatic AR tumor mutations of T878A, M750T, and D891H. In some embodiments, the prostate cancer comprises at least the three somatic AR tumor mutations at T878A, F877L, and V716M. In some embodiments, the prostate cancer comprises at least the two somatic AR tumor mutations of T878S and H875Y. In some embodiments, the prostate cancer comprises at least the two somatic AR tumor mutations of T878S and W742C. In some embodiments, the prostate cancer comprises at least the two somatic AR tumor mutations of W742C and W742L. In some embodiments, the prostate cancer comprises at least the two somatic AR tumor mutations of L702H and T878A.

In one aspect, the application pertains to a method of treating prostate cancer in a subpopulation of prostate cancer patients, comprising:
selecting a subject with prostate cancer for treatment, wherein the subject's prostate cancer comprises at least one somatic AR mutation; and
administering a therapeutically effective amount of a compound of Formula (I),

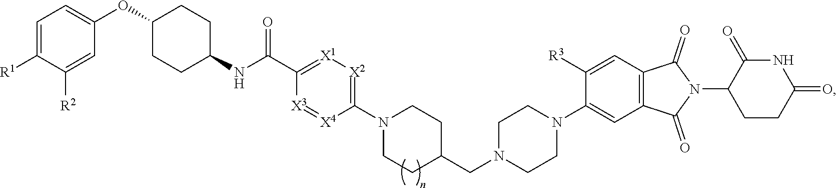

or a pharmaceutically acceptable salt, solvate, polymorph, isotopic derivative, or prodrug thereof, to the patient, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
   provided that at least two of X¹, X², X³, and X⁴ are CH.

In one aspect, the application pertains to a method of treating prostate cancer in a subpopulation of prostate cancer patients, comprising:
selecting a subject with prostate cancer for treatment, wherein the subject's prostate cancer comprises at least one somatic AR mutation; and
administering a therapeutically effective amount of a compound of Formula (I),

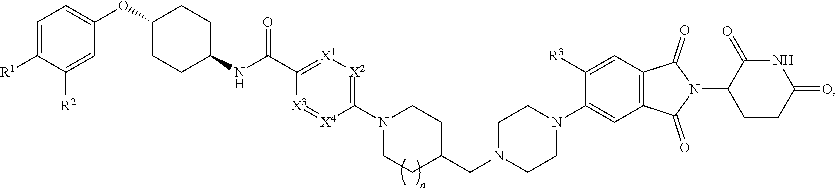

or a pharmaceutically acceptable salt, solvate, polymorph, isotopic derivative, or prodrug thereof, to the patient, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
wherein the therapeutically effective amount of the compound of Formula (I) is about 35 mg to about 1000 mg.

In one aspect, the application pertains to a method of treating prostate cancer in a subpopulation of prostate cancer patients, comprising:
selecting a prostate cancer patient for treatment based on the subject's somatic AR tumor biomarker status; and
administering a therapeutically effective amount of a compound of Formula (I),

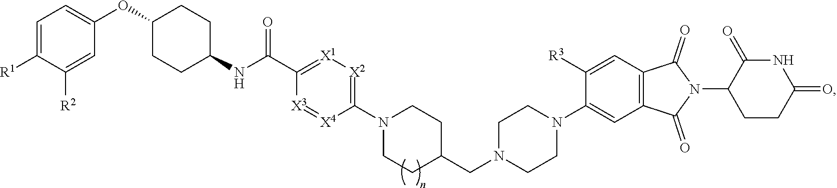

or a pharmaceutically acceptable salt, solvate, polymorph, isotopic derivative, or prodrug thereof, to the patient, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH; and
wherein the therapeutically effective amount of the compound of Formula (I) is about 35 mg to about 1000 mg.

In some embodiments, the AR biomarker status of a subject suffering from prostate cancer (e.g., mCRPC) can be determined through an analysis of the subject's circulating tumor DNA (ctDNA) (Ledet, E. M. et al. The Oncologist, 2019; 24:1-7, which is incorporated herein by reference in its entirety for all purposes). Alternative methods for determining the AR biomarker status of a subject suffering from prostate cancer include, without limitation, fluorescent in situ hybridization, immunohistochemistry, PCR analysis, or sequencing.

In some embodiments, the somatic AR tumor mutation of the prostate cancer can be determined through an analysis of the subject's circulating tumor DNA (ctDNA). Alternative methods for determining the somatic AR tumor mutation of the prostate cancer include, without limitation, fluorescent in situ hybridization, immunohistochemistry, PCR analysis, or sequencing.

In some embodiments, the AR biomarker status of a subject suffering from prostate cancer is determined in a blood sample derived from the subject.

In some embodiments, the AR biomarker status of a subject suffering from prostate cancer is determined in a solid biopsy derived from the tumor of the subject.

In some embodiments, the prostate cancer patient is selected for treatment based on the presence of at least one somatic AR tumor mutation.

In some embodiments, the prostate cancer patient is selected for treatment based on the presence of at least one somatic AR tumor mutation of L702H.

In some embodiments, the prostate cancer patient is selected for treatment based on the presence of at least one somatic AR tumor mutation of T878A.

In some embodiments, the prostate cancer patient is selected for treatment based on the presence of at least one somatic AR tumor mutation of H875Y.

In some embodiments, the prostate cancer patient is selected for treatment based on the presence of at least one somatic AR tumor mutation of H875L.

In some embodiments, the prostate cancer patient is selected for treatment based on the presence of at least one somatic AR tumor mutation of Q825E.

In some embodiments, the prostate cancer patient is selected for treatment based on the presence of at least one somatic AR tumor mutation of W742C.

In some embodiments, the prostate cancer patient is selected for treatment based on the presence of at least one somatic AR tumor mutation of W742L.

In some embodiments, the prostate cancer patient is selected for treatment based on the presence of at least one somatic AR tumor mutation of F877L.

In some embodiments, the prostate cancer patient is selected for treatment based on the presence of at least one somatic AR tumor mutation of T878S.

In some embodiments, the prostate cancer patient is selected for treatment based on the presence of at least one somatic AR tumor mutation of V716M.

In some embodiments, the prostate cancer patient is selected for treatment based on the presence of at least one somatic AR tumor mutation of D891H.

In some embodiments, the prostate cancer patient is selected for treatment based on the presence of at least one somatic AR tumor mutation of M750V.

In some embodiments, the prostate cancer patient is selected for treatment based on the presence of at least one somatic AR tumor mutation of M750T.

In one embodiment, the prostate cancer patient is selected for treatment based on the presence of at least one somatic AR tumor mutation of S889G.

In some embodiments, the prostate cancer patient is selected for treatment based on the presence of at least two somatic AR tumor mutations.

In some embodiments, the prostate cancer patient is selected for treatment based on the presence of at least two somatic AR tumor mutations of L702H and H875Y.

In some embodiments, the prostate cancer patient is selected for treatment based on the presence of at least three somatic AR tumor mutations of L702H, T878A, and H875Y.

In some embodiments, the prostate cancer patient is selected for treatment based on the presence of at least two somatic AR tumor mutations of T878A, and H875Y.

In some embodiments, the prostate cancer patient is selected for treatment based on the presence of at least four somatic AR tumor mutations of T878A, F877L, L702H, and V716M.

In some embodiments, the prostate cancer patient is selected for treatment based on the presence of at least three somatic AR tumor mutations of T878A, F877L, and V716M.

In some embodiments, the prostate cancer patient is selected for treatment based on the presence of at least two somatic AR tumor mutations of T878S and H875Y.

In some embodiments, the prostate cancer patient is selected for treatment based on the presence of at least two somatic AR tumor mutations of H875L and Q825E.

In some embodiments, the prostate cancer patient is selected for treatment based on the presence of at least two somatic AR tumor mutations of T878A and T878S.

In some embodiments, the prostate cancer patient is selected for treatment based on the presence of at least two somatic AR tumor mutations of T878S and W742C.

In some embodiments, the prostate cancer patient is selected for treatment based on the presence of at least two somatic AR tumor mutations of W742C and W742L.

In some embodiments, the prostate cancer patient is selected for treatment based on the presence of at least two somatic AR tumor mutations of L702H and T878A.

In one aspect, the application pertains to treating prostate cancer with a compound of Formula (I), wherein the compound of Formula (I) refers to a compound with the following structure:

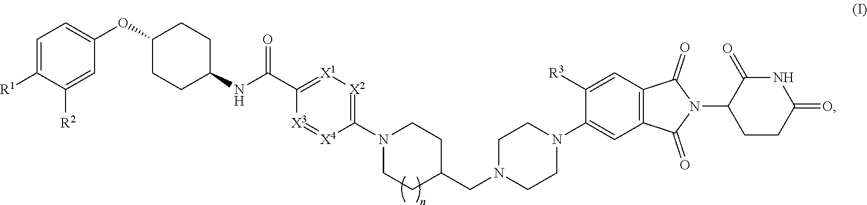

or a pharmaceutically acceptable salt, enantiomer, stereoisomer, solvate, polymorph, isotopic derivative, or prodrug thereof, wherein R¹, R², R³, X¹, X², X³, and X⁴ and n are defined herein. In some embodiments, the cancer is metastatic prostate cancer. In some embodiments, the cancer is castrate-resistant or castration-resistant prostate cancer. In some embodiments, the cancer is metastatic, castrate-resistant prostate cancer.

In one aspect, the application pertains to treating prostate cancer with a compound of Formula (I), wherein the compound of Formula (I) is selected from the group consisting of:

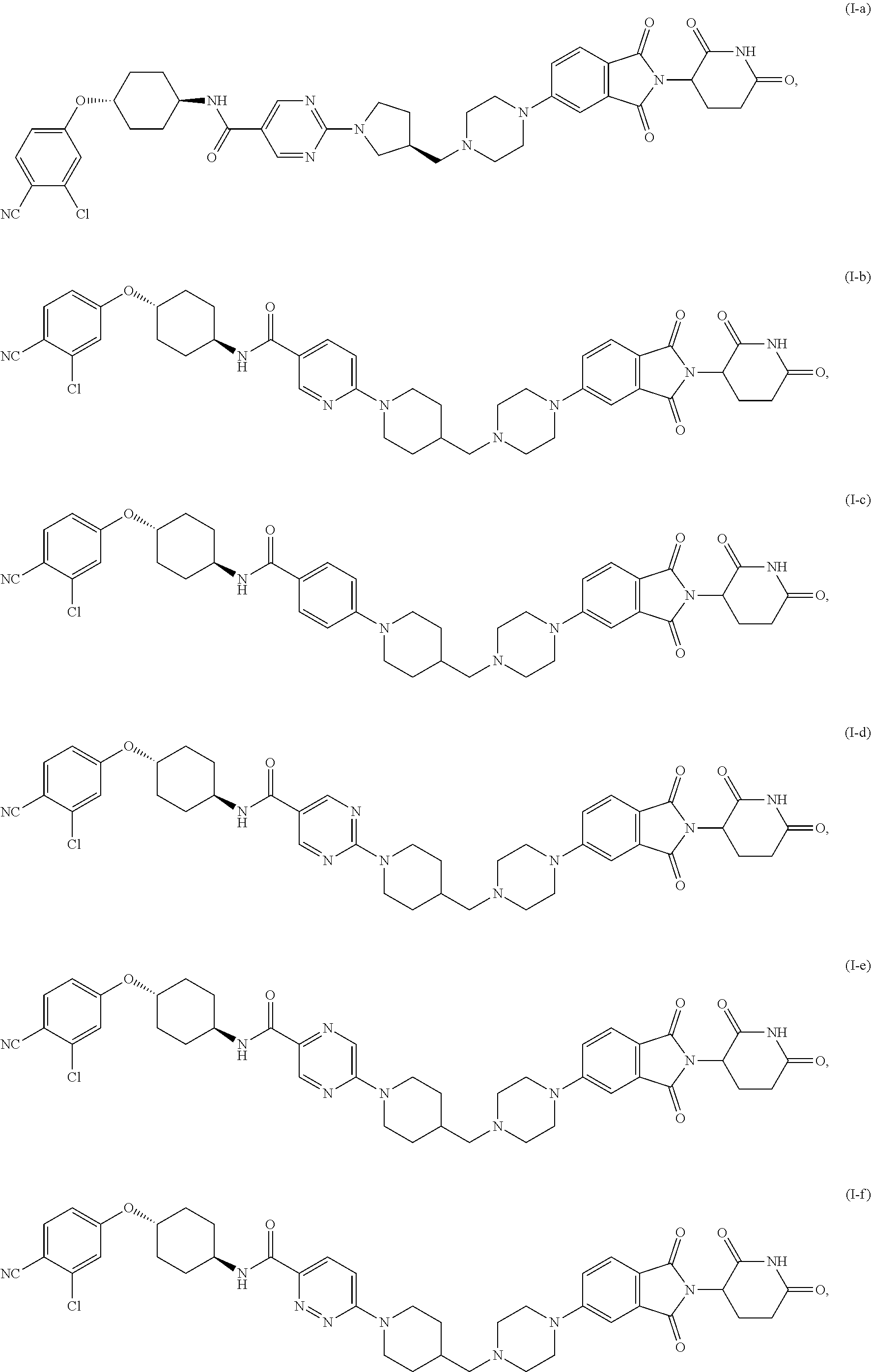

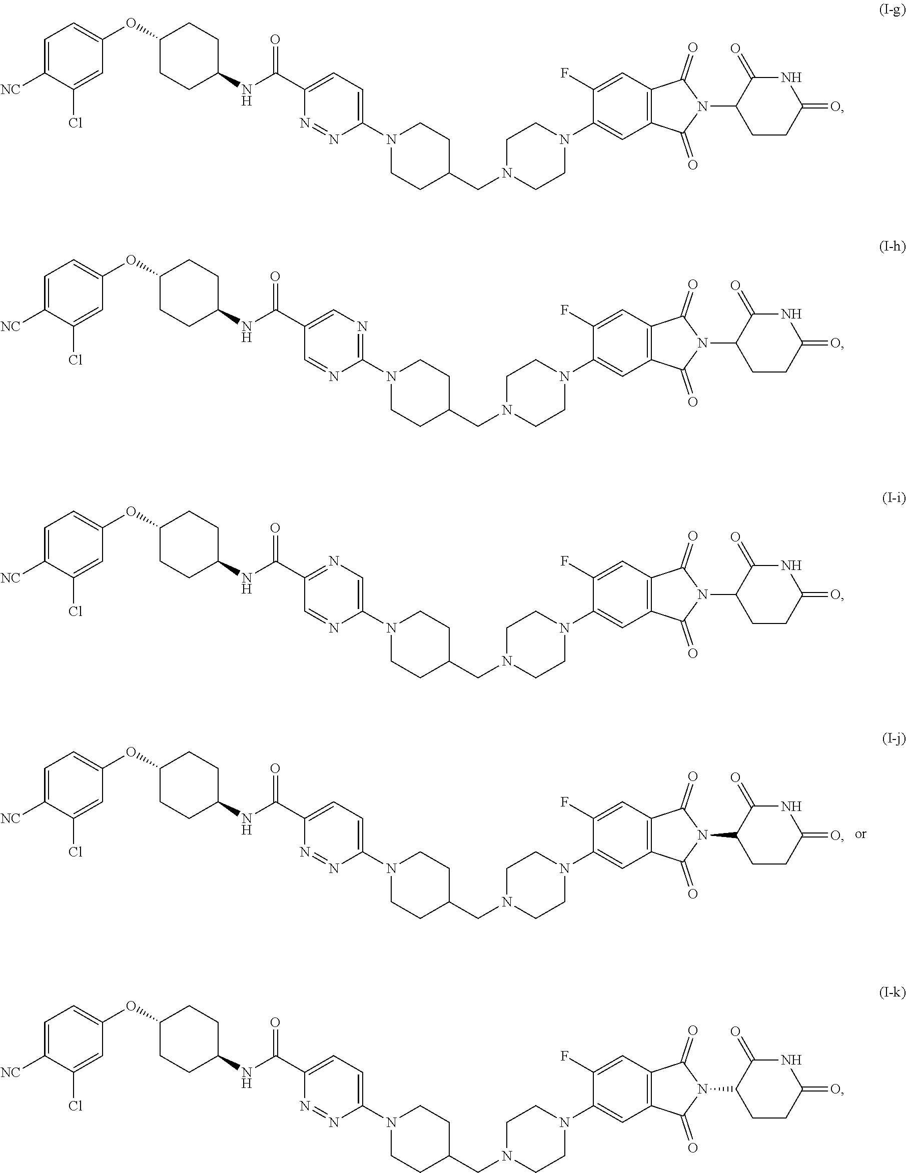

or a pharmaceutically acceptable salt, enantiomer, stereoisomer, solvate, polymorph, isotopic derivative, or prodrug thereof.

In one aspect, the application pertains to treating prostate cancer with a compound of Formula (I), wherein the compound of Formula (I) is selected from the group consisting of:

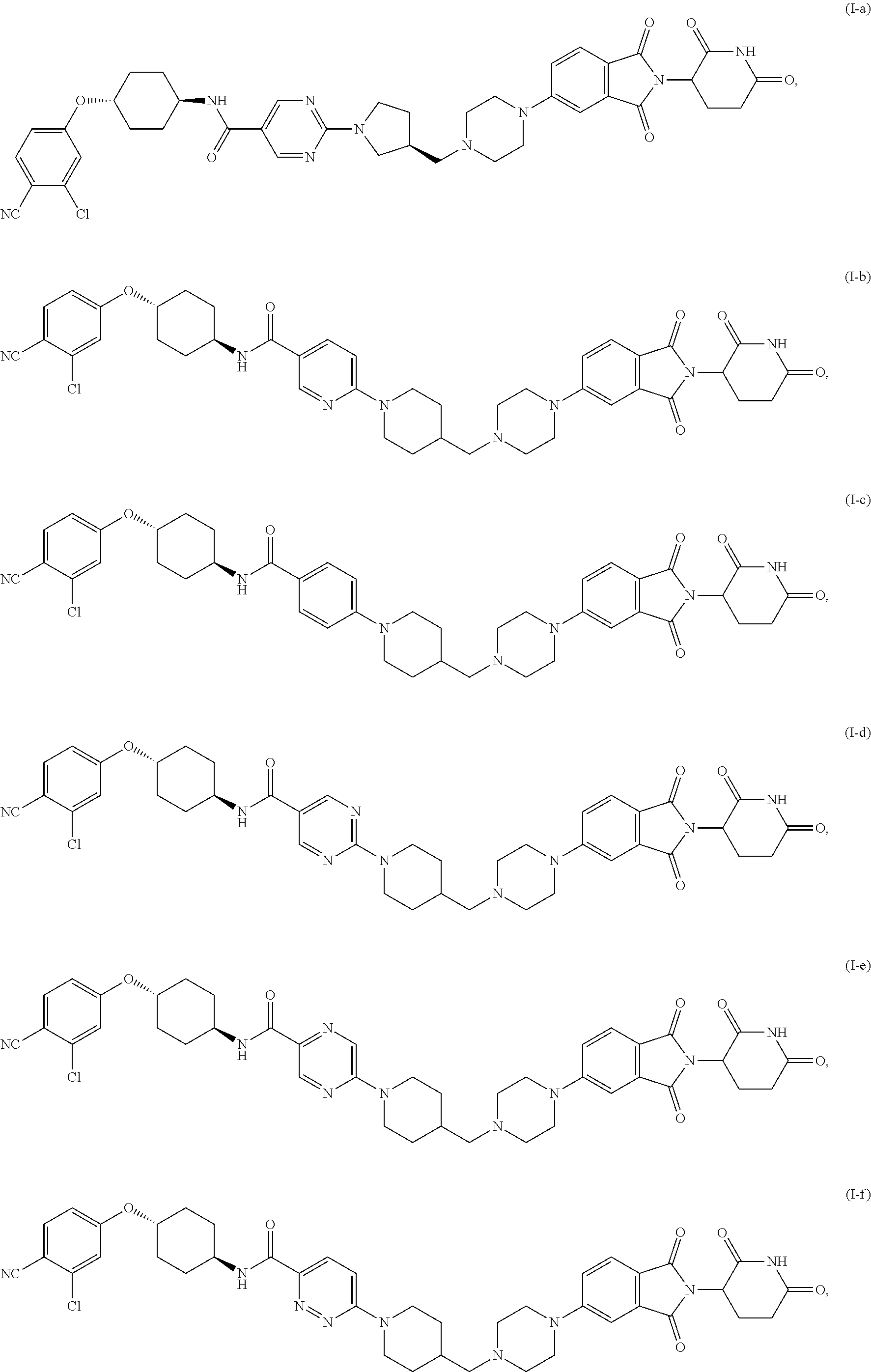

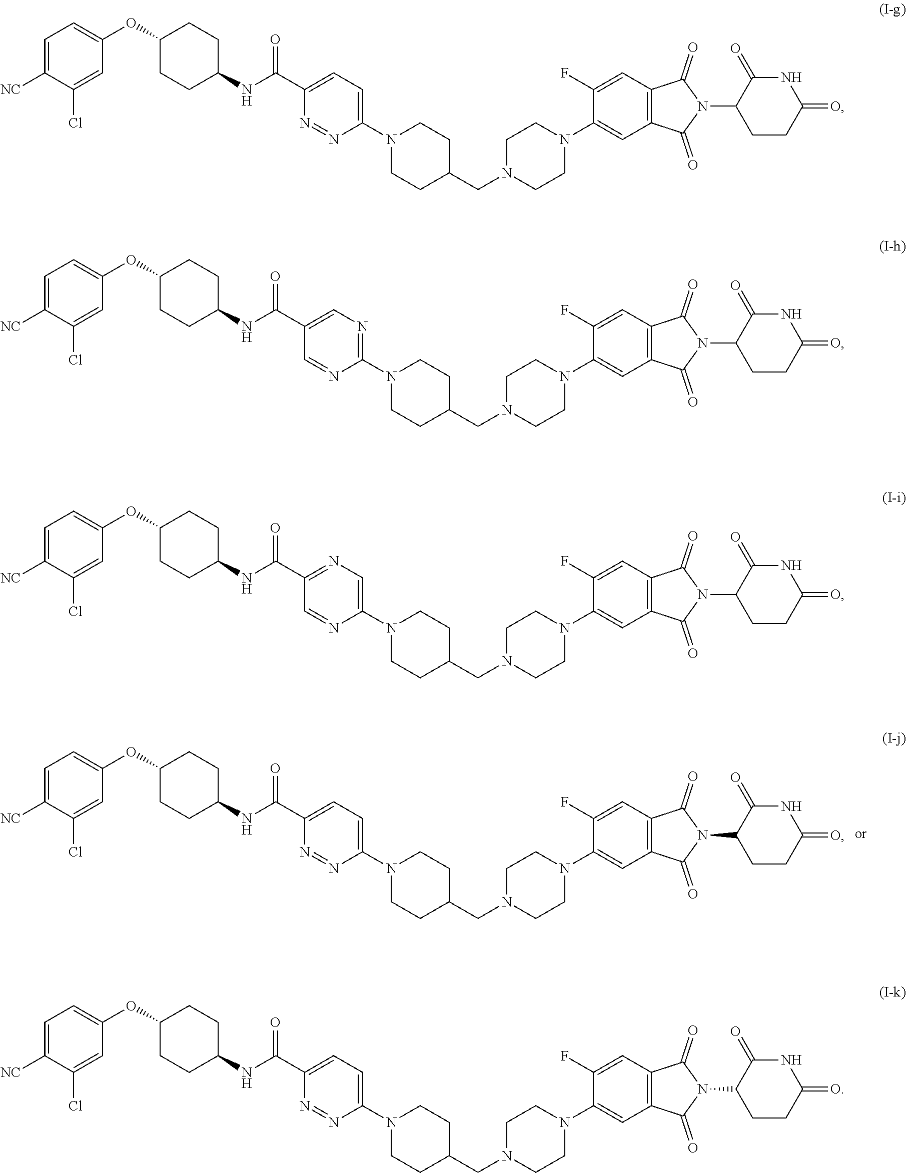

In some embodiments, the cancer is metastatic prostate cancer. In some embodiments, the prostate cancer is castrate-resistant or castration-resistant prostate cancer. In some embodiments, the prostate cancer is metastatic, castrate-resistant prostate cancer.

In one aspect, the application pertains to treating prostate cancer with a compound of Formula (I) in combination with another anti-cancer agent, wherein the compound of Formula (I) refers to a compound with the following structure:

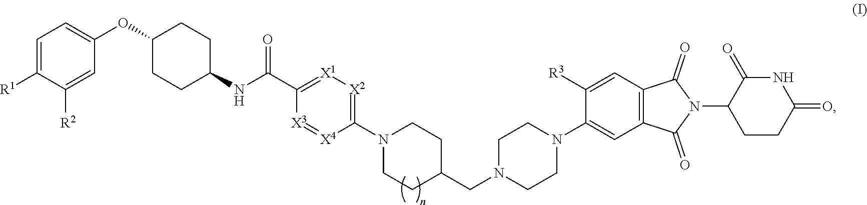

or a pharmaceutically acceptable salt, enantiomer, stereoisomer, solvate, polymorph, isotopic derivative, or prodrug thereof, wherein R¹, R², R³, X¹, X², X³, and X⁴ and n are defined herein. In some embodiments, the compound of Formula (I) is the compound of Formula (I-g). In some embodiments, the compound of Formula (I) is the compound of Formula (I-j).
In some embodiments, the compound of Formula (I) is the compound of Formula (I-k).

In some embodiments, the prostate cancer treated with the combination of a compound of Formula (I) and another anti-cancer agent is metastatic prostate cancer. In some embodiments, the prostate cancer treated with the combination of a compound of Formula (I) and another anti-cancer agent is castrate-resistant or castration-resistant prostate cancer. In some embodiments, the prostate cancer treated with the combination of a compound of Formula (I) and another anti-cancer agent is metastatic, castrate-resistant prostate cancer. In some embodiments, the other anti-cancer agent is abiraterone or a pharmaceutically acceptable salt thereof. In some embodiments, the other anti-cancer agent is abiraterone acetate.

In one aspect, treating cancer results in a reduction in size of a tumor. A reduction in size of a tumor may also be referred to as “tumor regression.” Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. In a preferred aspect, size of a tumor may be measured as a diameter of the tumor.

In another aspect, treating cancer results in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In another aspect, treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. In a preferred aspect, number of tumors may be measured by counting tumors visible to the naked eye or at a specified magnification. In a preferred aspect, the specified magnification is 2×, 3×, 4×, 5×, 10×, or 50×.

In another aspect, treating cancer results in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. In a preferred aspect, the number of metastatic lesions may be measured by counting metastatic lesions visible to the naked eye or at a specified magnification. In a preferred aspect, the specified magnification is 2×, 3×, 4×, 5×, 10×, or 50×.

In another aspect, treating cancer results in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with an active agent or compound.

In another aspect, treating cancer results in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. In a preferred aspect, an increase in average survival time of a population may be measured, for example, by calculating for a population the average length of survival following initiation of treatment with an active agent or compound. In another preferred aspect, an increase in average survival time of a population may also be measured, for example, by calculating for a population the average length of survival following completion of a first round of treatment with a compound of Formula (I).

In another aspect, treating cancer results in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. In a preferred aspect, tumor growth rate is measured according to a change in tumor diameter per unit time.

In another aspect, treating cancer results in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. In a preferred aspect, tumor regrowth is measured, for example, by measuring an increase in the diameter of a tumor after a prior tumor shrinkage that followed treatment. In another preferred aspect, a decrease in tumor regrowth is indicated by failure of tumors to reoccur after treatment has stopped.

The dosages of a compound of Formula (I) for any of the methods and uses described herein vary depending on the agent, the age, weight, and clinical condition of the recipient subject, and the experience and judgment of the clinician or practitioner administering the therapy, among other factors affecting the selected dosage.

The therapeutically effective amount of a compound of Formula (I) may be administered one or more times over a day for up to 30 or more days, followed by 1 or more days of non-administration of a compound of Formula (I). This type of treatment schedule, i.e., administration of a compound of Formula (I) on consecutive days followed by non-administration of a compound of Formula (I) on consecutive days may be referred to as a treatment cycle. A treatment cycle may be repeated as many times as necessary to achieve the intended affect.

In some embodiments, the therapeutically effective amount of a compound of Formula (I) is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer.

In some embodiments, the therapeutically effective amount of a compound of Formula (I) is about 10 to about 40 mg, about 20 to about 50 mg, about 30 to about 60 mg, about 40 to about 70 mg, about 50 to about 80 mg, about 60 to about 90 mg, about 70 to about 100 mg, about 80 to about 110 mg, about 90 to about 120 mg, about 100 to about 130 mg, about 110 to about 140 mg, about 120 to about 150 mg, about 130 to about 160 mg, about 140 to about 170 mg, about 150 to about 180 mg, about 160 to about 190 mg, about 170 to about 200 mg, about 180 to about 210 mg, about 190 to about 220 mg, about 200 to about 230 mg, about 210 to about 240 mg, about 220 to about 250 mg, about 230 to about 260 mg, about 240 to about 270 mg, about 250 to about 280 mg, about 260 to about 290 mg, about 270 to about 300 mg, about 280 to about 310 mg, about 290 to about 320 mg, about 300 to about 330 mg, about 310 to about 340 mg, about 320 to about 350 mg, about 330 to about 360 mg, about 340 to about 370 mg, about 350 to about 380 mg, about 360 to about 390 mg, about 370 to about 400 mg, about 380 to about 410 mg, about 390 to about 420 mg, about 400 to about 430 mg, about 410 to about 440 mg, about 420 to about 450 mg, about 430 to about 460 mg, about 440 to about 470 mg, about 450 to about 480 mg, about 460 to about 490 mg, about 470 to about 500 mg, about 480 to about 510 mg, about 490 to about 520 mg, about 500 to about 530 mg, about 510 to about 540 mg, about 520 to about 550 mg, about 530 to about 560 mg, about 540 to about 570 mg, about 550 to about 580 mg, about 560 to about 590 mg, about 570 to about 600 mg, about 580 to about 610 mg, about 590 to about 620 mg, about 600 to about 630 mg, about 610 to about 640 mg, about 620 to about 650 mg, about 630 to about 660 mg, about 640 to about 670 mg, about 650 to about 680 mg, about 660 to about 690 mg, about 670 to about 700 mg, about 680 to about 710 mg, about 690 to about 720 mg, about 700 to about 730 mg, about 710 to about 740 mg, about 720 to about 750 mg, about 730 to about 760 mg, about 740 to about 770 mg, about 750 to about 780 mg, about 760 to about 790 mg, about 770 to about 800 mg, about 780 to about 810 mg, about 790 to about 820 mg, about 800 to about 830 mg, about 810 to about 840 mg, about 820 to about 850 mg, about 830 to about 860 mg, about 840 to about 870 mg, about 850 to about 880 mg, about 860 to about 890 mg, about 870 to about 900 mg, about 880 to about 910 mg, about 890 to about 920 mg, about 900 to about 930 mg, about 910 to about 940 mg, about 920 to about 950 mg, about 930 to about 960 mg, about 940 to about 970 mg, about 950 to about 980 mg, about 960 to about 990 mg, or about 970 to about 1,000 mg administered once, twice, three times, four times, or more daily in single or divided doses (which dose may be adjusted for the patient's weight in kg, body surface area in m², and age in years).

In some embodiments, the therapeutically effective amount of a compound of Formula (I) is about 35 mg to about 1000 mg. In some embodiments, the therapeutically effective amount of a compound of Formula (I) is about 35 mg to about 1000 mg administered once, twice, three times, four times, or more daily in single or divided doses (which dose may be adjusted for the patient's weight in kg, body surface area in m², and age in years).

In some embodiments, the therapeutically effective amount of a compound of Formula (I) is about 70 mg to about 1000 mg administered once, twice, three times, four times, or more daily in single or divided doses (which dose may be adjusted for the patient's weight in kg, body surface area in m², and age in years).

In some embodiments, the therapeutically effective amount of a compound of Formula (I) is about 35 mg, 70 mg, 105 mg, 140 mg, 175 mg, 210 mg, 245 mg, 280 mg, 315 mg, 350 mg, 385 mg, 420 mg, 455 mg, 490 mg, 525 mg, 560 mg, 595 mg, 630 mg, 665 mg, or 700 mg administered once, twice, three times, four times, or more daily in single or divided doses (which dose may be adjusted for the patient's weight in kg, body surface area in m², and age in years).

The therapeutically effective amount of a compound of Formula (I) can also range from about 0.01 mg/kg per day to about 100 mg/kg per day. In an aspect, therapeutically effective amount of a compound of Formula (I) can range from about 0.05 mg/kg per day to about 10 mg/kg per day. In an aspect, therapeutically effective amount of a compound of Formula (I) can range from about 0.075 mg/kg per day to about 5 mg/kg per day. In an aspect, therapeutically effective amount of a compound of Formula (I) can range from about 0.10 mg/kg per day to about 1 mg/kg per day. In an aspect, therapeutically effective amount of a compound of Formula (I) can range from about 0.20 mg/kg per day to about 0.70 mg/kg per day.

In some embodiments, the therapeutically effective amount of a compound of Formula (I) is about 0.10 mg/kg per day, about 0.15 mg/kg per day, about 0.20 mg/kg per day, about 0.25 mg/kg per day, about 0.30 mg/kg per day, about 0.35 mg/kg per day, about 0.40 mg/kg per day, about 0.45 mg/kg per day, about 0.50 mg/kg per day, about 0.55 mg/kg per day, about 0.60 mg/kg per day, about 0.65 mg/kg per day, about 0.70 mg/kg per day, about 0.75 mg/kg per day, about 0.80 mg/kg per day, about 0.85 mg/kg per day, about 0.90 mg/kg per day, about 0.95 mg/kg per day, or about 1.00 mg/kg per day.

In some embodiments, the therapeutically effective amount of a compound of Formula (I) is about 1.05 mg/kg per day, about 1.10 mg/kg per day, about 1.15 mg/kg per day, about 1.20 mg/kg per day, about 1.25 mg/kg per day, about 1.30 mg/kg per day, about 1.35 mg/kg per day, about 1.40 mg/kg per day, about 1.45 mg/kg per day, about 1.50 mg/kg per day, about 1.55 mg/kg per day, about 1.60 mg/kg per day, about 1.65 mg/kg per day, about 1.70 mg/kg per day, about 1.75 mg/kg per day, about 1.80 mg/kg per day, about 1.85 mg/kg per day, about 1.90 mg/kg per day, about 1.95 mg/kg per day, or about 2.00 mg/kg per day.

In some embodiments, the therapeutically effective amount of a compound of Formula (I) is about 2 mg/kg per day, about 2.5 mg/kg per day, about 3 mg/kg per day, about 3.5 mg/kg per day, about 4 mg/kg per day, about 4.5 mg/kg per day, about 5 mg/kg per day, about 5.5 mg/kg per day, about 6 mg/kg per day, about 6.5 mg/kg per day, about 7 mg/kg per day, about 7.5 mg/kg per day, about 8.0 mg/kg per day, about 8.5 mg/kg per day, about 9.0 mg/kg per day, about 9.5 mg/kg per day, or about 10 mg/kg per day.

In some embodiments, the therapeutically effective amount of a compound of Formula (I) is administered to the subject once daily. In some embodiments, this daily dose of a compound of Formula (I) may administered to the subject all at once. In some embodiments, this daily dose of a compound of Formula (I) may administered to the subject in two portions (a divided dose). In some embodiments, this daily dose of a compound of Formula (I) may administered to the subject in three portions. In some embodiments, this daily dose of a compound of Formula (I) may administered to the subject in four portions. In some embodiments, this daily dose of a compound of Formula (I) may administered to the subject in five or more portions. In some embodiments, these portions are administered to the subject at regular intervals throughout the day, for example, every 12 hours, every 8 hours, every 6 hours, every 5 hours, every 4 hours, etc.

In some embodiments, the therapeutically effective amount of the compound of Formula (I) results in a mean day 15 AUC₀₋₂₄ of greater than about 3,500 ng*hr/mL, about 3,550 ng*hr/mL, about 3,600 ng*hr/mL, about 3,650 ng*hr/mL, about 3,700 ng*hr/mL, about 3,750 ng*hr/mL, about 3,800 ng*hr/mL, about 3,850 ng*hr/mL, about 3,900 ng*hr/mL, about 3,950 ng*hr/mL, about 4,000 ng*hr/mL, 4,050 ng*hr/mL, about 4,100 ng*hr/mL, about 4,150 ng*hr/mL, about 4,200 ng*hr/mL, 4,250 ng*hr/mL, about 4,300 ng*hr/mL, about 4,350 ng*hr/mL, about 4,400 ng*hr/mL, about 4,450 ng*hr/mL, about 4,500 ng*hr/mL, about 4,550 ng*hr/mL, about 4,600 ng*hr/mL, about 4,650 ng*hr/mL, about 4,700 ng*hr/mL, about 4,750 ng*hr/mL, about 4,800 ng*hr/mL, about 4,850 ng*hr/mL, about 4,900 ng*hr/mL, about 4,950 ng*hr/mL, about 5,000 ng*hr/mL, 5,050 ng*hr/mL, about 5,100 ng*hr/mL, about 5,150 ng*hr/mL, about 5,200 ng*hr/mL, about 5,250 ng*hr/mL, about 5,300 ng*hr/mL, about 5,350 ng*hr/mL, about 5,400 ng*hr/mL, about 5,450 ng*hr/mL, about 5,500 ng*hr/mL, about 5,550 ng*hr/mL, about 5,600 ng*hr/mL, about 5,650 ng*hr/mL, about 5,700 ng*hr/mL, about 5,750 ng*hr/mL, about 5,800 ng*hr/mL, about 5,850 ng*hr/mL, about 5,900 ng*hr/mL, 5,950 ng*hr/mL, or about 6,000 ng*hr/mL, 6,050 ng*hr/mL, about 6,100 ng*hr/mL, about 6,150 ng*hr/mL, about 6,200 ng*hr/mL, about 6,250 ng*hr/mL, about 6,300 ng*hr/mL, about 6,350 ng*hr/mL, about 6,400 ng*hr/mL, about 6,450 ng*hr/mL, about 6,500 ng*hr/mL, about 6,550 ng*hr/mL, about 6,600 ng*hr/mL, about 6,650 ng*hr/mL, about 6,700 ng*hr/mL, about 6,750 ng*hr/mL, about 6,800 ng*hr/mL, about 6,850 ng*hr/mL, about 6,900 ng*hr/mL, 6,950 ng*hr/mL, or about 7,000 ng*hr/mL, 7,050 ng*hr/mL, about 7,100 ng*hr/mL, about 7,150 ng*hr/mL, about 7,200 ng*hr/mL, about 7,250 ng*hr/mL, about 7,300 ng*hr/mL, about 7,350 ng*hr/mL, about 7,400 ng*hr/mL, about 7,450 ng*hr/mL, about 7,500 ng*hr/mL, about 7,550 ng*hr/mL, about 7,600 ng*hr/mL, about 7,650 ng*hr/mL, about 7,700 ng*hr/mL, about 7,750 ng*hr/mL, about 7,800 ng*hr/mL, about 7,850 ng*hr/mL, about 7,900 ng*hr/mL, 7,950 ng*hr/mL, or about 8,000 ng*hr/mL, 8,050 ng*hr/mL, about 8,100 ng*hr/mL, about 8,150 ng*hr/mL, about 8,200 ng*hr/mL, about 8,250 ng*hr/mL, about 8,300 ng*hr/mL, about 8,350 ng*hr/mL, about 8,400 ng*hr/mL, about 8,450 ng*hr/mL, about 8,500 ng*hr/mL, about 8,550 ng*hr/mL, about 8,600 ng*hr/mL, about 8,650 ng*hr/mL, about 8,700 ng*hr/mL, about 8,750 ng*hr/mL, about 8,800 ng*hr/mL, about 8,850 ng*hr/mL, about 8,900 ng*hr/mL, 8,950 ng*hr/mL, or about 9,000 ng*hr/mL.

In some embodiments, the therapeutically effective amount of the compound of Formula (I) results in a mean day 15 Cₘₐₓ of greater than about 250 ng/mL, about 255 ng/mL, about 260 ng/mL, about 265 ng/mL, about 270 ng/mL, about 275 ng/mL, about 280 ng/mL, about 285 ng/mL, about 290 ng/mL, about 295 ng/mL, about 300 ng/mL, about 305 ng/mL, about 310 ng/mL, about 315 ng/mL, about 320 ng/mL, about 325 ng/mL, about 330 ng/mL, about 335 ng/mL, about 340 ng/mL, about 345 ng/mL, about 350 ng/mL, about 355 ng/mL, about 360 ng/mL, about 365 ng/mL, about 370 ng/mL, about 375 ng/mL, about 380 ng/mL, about 385 ng/mL, about 390 ng/mL, about 395 ng/mL, about 400 ng/mL, about 405 ng/mL, about 410 ng/mL, about 415 ng/mL, about 420 ng/mL, about 425 ng/mL, about 430 ng/mL, about 435 ng/mL, about 440 ng/mL, about 445 ng/mL, about 450 ng/mL, about 455 ng/mL, about 460 ng/mL, about 465 ng/mL, about 470 ng/mL, about 475 ng/mL, about 480 ng/mL, about 485 ng/mL, about 490 ng/mL, about 495 ng/mL, or about 500 ng/mL.

The therapeutically effective amount of a compound of Formula (I) can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of a compound of Formula (I) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

In some embodiments, for the methods of treating prostate cancer with the combination of a compound of Formula (I) and another anti-cancer agent, the therapeutically effective amount of a compound of Formula (I) is described herein, and the therapeutically effective amount of the other anti-cancer agent is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer. In some embodiments, the other anti-cancer agent is abiraterone or a pharmaceutically acceptable salt thereof. In some embodiments, the other anti-cancer agent is abiraterone acetate.

In some embodiments, for the methods of treating prostate cancer with the combination of a compound of Formula (I) and abiraterone, or a pharmaceutically acceptable salt thereof, the therapeutically effective amount of a compound of Formula (I) is described herein, and the therapeutically effective amount of abiraterone, or a pharmaceutically acceptable salt thereof, is 0.01, 0.05, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 935, 940, 945, 950, 955, 960, 965, 970, 975, 980, 985, 990, 995, or 1,000 mg administered once, twice, three times, four times, or more daily for one, two, three, four, five, six, seven, eight, nine, ten, eleven, twelve, thirteen, fourteen, fifteen, thirty consecutive days, or, once, twice, three times, four times, or more daily, in single or divided doses, for 2 months, 3 months, 4 months, 5 months, 6 months, or longer. In some embodiments, the abiraterone is abiraterone acetate.

In some embodiments, for the methods of treating prostate cancer with the combination of a compound of Formula (I) and abiraterone acetate, the therapeutically effective amount of a compound of Formula (I) is described herein, and the therapeutically effective amount of abiraterone acetate is 1,000 mg administered orally once daily for one, two, three, four, five, six, seven, eight, nine, ten, eleven, twelve, thirteen, fourteen, fifteen, thirty, or more consecutive days, in single or divided doses. In some embodiments, the abiraterone acetate is administered in combination with 5 mg of prednisone administered orally, twice daily. In some embodiments, the combination of the compound of Formula (I) and abiraterone acetate is administered to the subject in need thereof in the fasted state. In some embodiments, the subject does not eat for at least two hours before, and at least one hour after, the administration of the combination of the compound of Formula (I) and abiraterone acetate.

In some embodiments, the compound of Formula (I) and the other anti-cancer agent are administered to the subject simultaneously. In some embodiments, the compound of Formula (I) and the other anti-cancer agent are administered to the subject sequentially.

In some embodiments, the compound of Formula (I) and the other anti-cancer agent are administered to the subject in temporal proximity.

In some embodiments, “temporal proximity” means that administration of compound of Formula (I) occurs within a time period before or after the administration of the other anti-cancer agent, such that the therapeutic effect of the compound of Formula (I) overlaps with the therapeutic effect of the other anti-cancer agent. In some embodiments, the therapeutic effect of the compound of Formula (I) completely overlaps with the therapeutic effect of the other anti-cancer agent. In some embodiments, “temporal proximity” means that administration of the compound of Formula (I) occurs within a time period before or after the administration of the other anti-cancer agent, such that there is a synergistic effect between the compound of Formula (I) and the other anti-cancer agent.

“Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

### Pharmaceutical Compositions

In some embodiments, a compound of Formula (I) is formulated for oral administration. For example, in some embodiments, a compound of Formula (I) is formulated as a tablet that comprises zero, one, two, or more of each of the following: emulsifier; surfactant, binder; disintegrant, glidant; and lubricant.

In some embodiments, the emulsifier is hypromellose.

In some embodiments, the surfactant is vitamin E polyethylene glycol succinate.

In some embodiments, the binder (also referred to herein as a filler) is selected from the group consisting of microcrystalline cellulose, lactose monohydrate, sucrose, glucose, and sorbitol.

In some embodiments, the disintegrant is croscarmellose sodium.

In some embodiments, the glidant refers to a substance used to promote powder flow by reducing interparticle cohesion. In some embodiments, in the dosage forms of the disclosure, the glidant is selected from the group consisting of silicon dioxide, silica colloidal anhydrous, starch, and talc.

In some embodiments, the lubricant refers to a substance that prevents ingredients from sticking and/or clumping together in the machines used in preparation of the dosage forms of the disclosure. In some embodiments, in the dosage forms of the disclosure, the lubricant is selected from the group consisting of magnesium stearate, sodium stearyl fumarate, stearic acid, and vegetable stearin.

The pharmaceutical compositions containing a compound of Formula (I) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of a compound of Formula (I) into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating a compound of Formula (I) in the required amount in an appropriate solvent with one or a combination of ingredients enumerated above, as required, followed by filtered sterilization. Generally, dispersions are prepared by incorporating the active agent or compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, a compound of Formula (I) can be incorporated with excipients and used in the form of tablets, troches, or capsules. Oral compositions can also be prepared using a fluid carrier for use as a mouthwash, wherein the agent or compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, sodium starch glycolate (Primojel®), or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

For administration by inhalation, the agents or compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active agents or compounds are formulated into ointments, salves, gels, or creams as generally known in the art.

In one aspect, a compound of Formula (I) is prepared with pharmaceutically acceptable carriers that will protect the agent or compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form, or “unit doses,” as used herein refers to physically discrete units suited as unitary dosages for the subject to be treated; each unit containing a predetermined quantity of active agent or compound calculated to produce the desired therapeutic effect in association with the required pharmaceutical carrier. The specification for the dosage unit forms of the application are dictated by and directly dependent on the unique characteristics of a compound of Formula (I) and the particular therapeutic effect to be achieved.

The pharmaceutical compositions can be included in a container, pack, or dispenser together with instructions for administration.

Illustrative modes of administration for a compound of Formula (I) includes systemic or local administration such as oral, nasal, parenteral, transdermal, subcutaneous, vaginal, buccal, rectal or topical administration modes. In some embodiments, the compound of Formula (I), or a pharmaceutically acceptable salt or hydrate thereof, is administered orally. In some embodiments, the compound of Formula (I) is administered as a tablet, capsule, caplet, solution, suspension, syrup, granule, bead, powder, or pellet.

Illustrative pharmaceutical compositions are tablets and gelatin capsules comprising a salt of compound of Formula (I) and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the salt such as cyclodextrin, hydroxypropyl-cyclodextrin, PEG400, and/or PEG200.

For preparing pharmaceutical compositions from a compound of Formula (I), or a salt or hydrate thereof, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions and emulsions. For example, water or water-propylene glycol solutions for parenteral injection or addition of sweeteners and opacifiers for oral solutions, suspensions and emulsions. Liquid form preparations may also include solutions for intranasal administration.

Liquid, particularly injectable, compositions can, for example, be prepared by dissolution, dispersion, etc. For example, the disclosed salt is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Aerosol preparations suitable for inhalation may include solutions and solids in powder form, which may be in combination with a pharmaceutically acceptable carrier, such as an inert compressed gas, e.g., nitrogen.

Also included are solid form preparations that are intended to be converted, shortly before use, to liquid form preparations for either oral or parenteral administration. Such liquid forms include solutions, suspensions and emulsions.

Depending on the intended mode of administration, the disclosed compositions can be in solid, semi-solid or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, and all using forms well known to those skilled in the pharmaceutical arts.

Pharmaceutical compositions can be prepared according to conventional mixing, granulating or coating methods, respectively, and the present pharmaceutical compositions can contain from about 0.1% to about 99%, from about 5% to about 90%, or from about 1% to about 20% of the disclosed salt by weight or volume.

All amounts of any component of an oral dosage form described herein, e.g., a tablet, that are indicated based on % w/w refer to the total weight of the oral dosage form, unless otherwise indicated.

### EXAMPLES

The disclosure is further illustrated by the following examples, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### Example 1—In Vitro Studies with Compound (I-g)

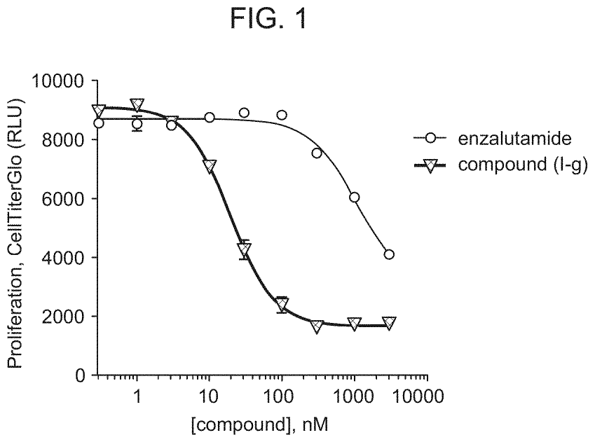

Compound (I-g) was shown to degrade 95% to 98% of androgen receptors (AR) in multiple cells lines typically used in prostate cancer research, including, for example, VCaP cells. (DC₅₀ in VCaP for Compound (I-g) is 1 nM.) Near-maximal degradation was observed within 4 hours of administration of Compound (I-g). Compound (I-g) inhibits VCaP proliferation about 60 times more potently than enzalutamide. (FIG. 1.)

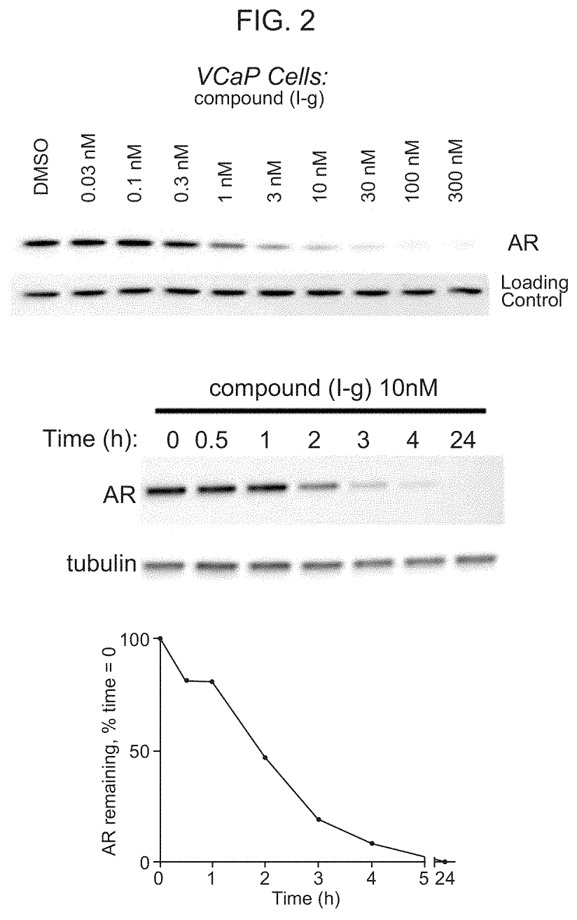

FIG. 2 shows the reduction of AR in VCaP tumor cells in response to treatment with Compound (I-g) at concentrations of 0.03 nM, 0.1 nM, 0.3 nM, 1 nM, 3 nM, 10 nM, 30 nM, 100 nM, and 300 nM.

### Example 2—In Vivo Studies with Animals and Assessment of the Preclinical Efficacious Exposure Range for Compound (I-g)

Preclinical animal studies were performed with Compound (I-g) in VCaP xenograft animal models. VCaP was derived from a vertebral metastatic growth of a prostate carcinoma. It is a desirable cell line for in vivo studies as it exhibits many of the characteristics of clinical prostate carcinoma. VCaP is also a useful model to study AR resistance as it expresses AR splice variants that have been shown to drive resistance to AR antagonists. (European Urology. 2018 April; 73(4):572-582.)

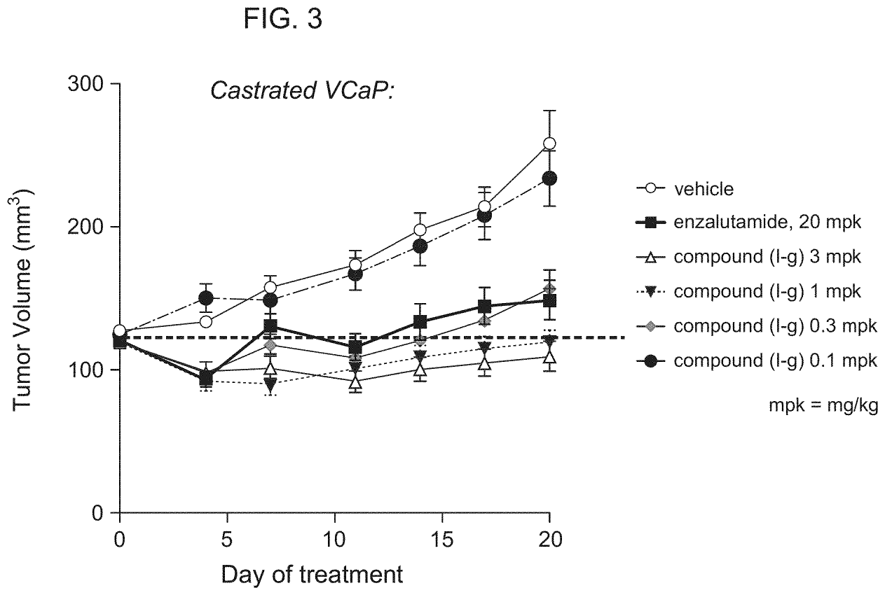

Oral, once daily administration of Compound (I-g) at doses of 0.1 mg/kg (mpk), 0.3 mg/kg, 1 mg/kg, and 3 mg/kg were performed in a castrated VCaP xenograft model (FIG. 3). Enzalutamide (20 mg/kg) and vehicle were also used as control groups.

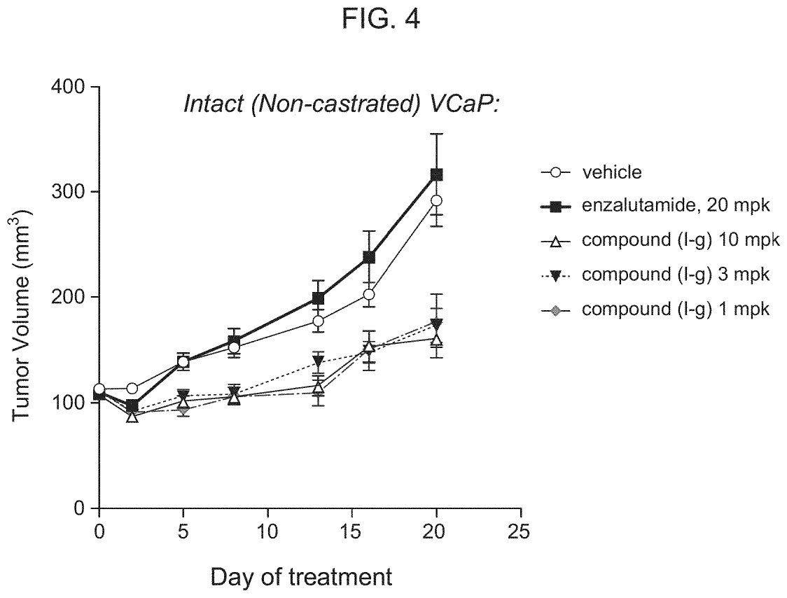

Oral, once daily administration of Compound (I-g) at doses of 1 mg/kg, 3 mg/kg, 10 mg/kg were performed in an intact (non-castrated) VCaP xenograft model (FIG. 4). Enzalutamide (20 mg/kg) and vehicle were also used as control groups.

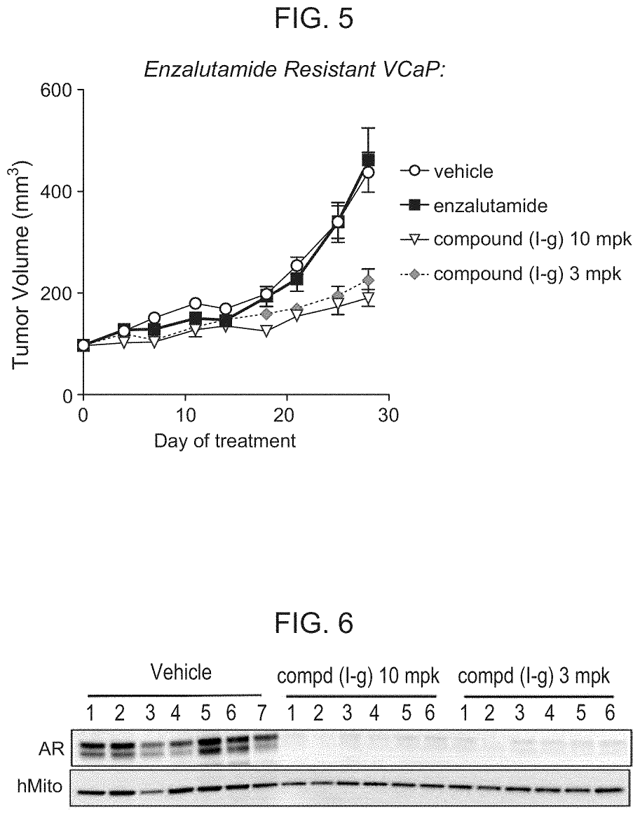

Oral, once daily administration of Compound (I-g) at doses of 3 mg/kg and 10 mg/kg were performed in an enzalutamide resistant VCaP xenograft model (FIG. 5). Enzalutamide (20 mg/kg) and vehicle were also used as control groups.

The pharmacokinetic results of oral, once daily administration of Compound (I-g) at doses of 1 mg/kg and 3 mg/kg are shown below in Table 1. A dose of 1 mg/kg of Compound (I-g) is the lowest dose that is superior to enzalutamide in a VCaP xenograft. A 3 mg/kg dose of Compound (I-g) was the lowest efficacious dose in an enzalutamide-resistant VCaP model (tumor growth inhibition of 70% compared to a control group).

FIG. 6 shows the reduction of AR in enzalutamide-resistant VCaP tumors in response to dosing with Compound (I-g) at 10 mg/kg and 3 mg/kg (oral, once daily).

TABLE 1

| Dose | Mean AUC₀₋₂₄ | Mean Cₘₐₓ |
|---|---|---|
| (oral, once daily) | (ng * hr/mL) ^{†} | (ng/mL) ^{‡} |
| 1 mg/kg | 3628 | 224 |
| 3 mg/kg | 8106 | 507 |

Values represent total drug concentrations
^{†} AUC or Area Under the Curve is a measurement of total exposure
^{‡} Cmax is a measurement of peak concentration during the dosing period

### Example 3—In Vivo Animal Studies with Compound (I-g) and Abiraterone

The combination of Compound (I-g) and abiraterone attenuated tumor growth more significantly than either agent alone in castrated VCaP xenografts.

### Example 4—Toxicology Studies

Animals were orally administered compound (I-g) once daily for 28 days, followed by a 14-day recovery for high-dose animals.

In dogs, once daily, oral doses of 3 mg/kg, 10 mg/kg, or 30 mg/kg of Compound (I-g) were administered. It was determined that the 30 mg/kg dose exceeded the maximum tolerated dose. Gastrointestinal alterations were observed at all dose levels (including vehicle alone). Reversible liver function enzyme elevation, which is considered non-adverse, was observed in some mid- and high-dose animals. Male animals exhibited decreased prostate weights, which may be attributable to the pharmacology of Compound (I-g).

In rats, males were administered once daily, oral doses of Compound (I-g) at doses of 20 mg/kg, 60 mg/kg, or 120 mg/kg. Female rats were administered once daily, oral doses of Compound (I-g) at doses of 20 mg/kg, 40 mg/kg, or 120 mg/kg.

Overall, Compound (I-g) was well tolerated at all doses, with the exception of the 80 mg/kg female cohort. These rats lost body weight and consumed less food. All of the findings in male high-dose rats were fully reversible (liver hypertrophy, femur physis thickening). Male rats also exhibited decreased prostate weights, which may be attributable to the pharmacology of Compound (I-g).

### Example 5—Phase I Clinical Trial Study Design with Compound (I-g)

A Phase I Clinical Trial with Compound (I-g) was undertaken. A traditional 3+3 dose escalation design was implemented. Starting dose of Compound (I-g) was 35 mg administered orally, once daily with food. Dose increases were dependent upon toxicities.

The key criteria for this trial were: men with metastatic, castrate-resistant prostate cancer (mCRPC); at least two prior systemic therapies, at least one of which was abiraterone or enzalutamide; and disease progression on most recent therapy (for example, rising PSA or two or more new lesions upon bone scan).

The key objectives for this trial were obtaining the maximum tolerated dose of Compound (I-g) and the recommended Phase II trial dose. Additional objectives included assessing overall safety of Compound (I-g), pharmacokinetics, anti-tumor activity (for example, PSA, RECIST), and biomarkers, including, for example, AR degradation in CTCs and pre- vs. post-treatment biopsies (when available); AR (and other) gene mutations, amplifications in ctDNA; and AR-V7 in CTCs.

### Example 6—Phase I Pharmacokinetic Data—Oral Administration of Compound (I-g)

In a Phase I clinical trial, Compound (I-g) was administered orally at a dose of 35 mg/day, 70 mg/day, and 140 mg/day. It was observed that treatment with 140 mg/day dose of Compound (I-g) enters the preclinical efficacious range associated with tumor growth inhibition.

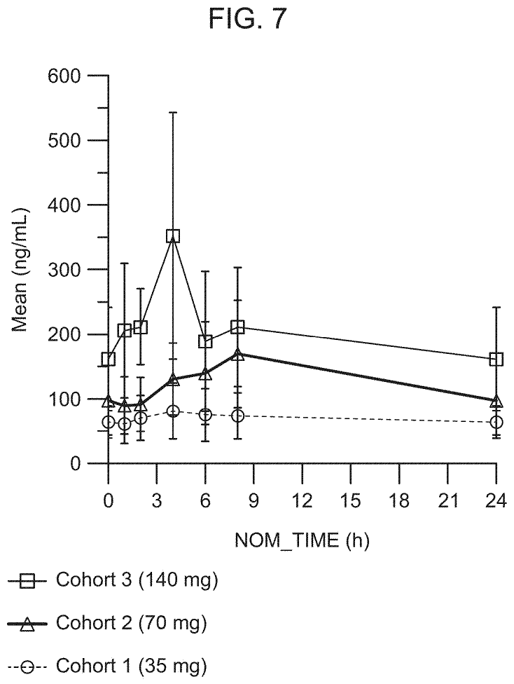

The initial pharmacokinetic results are shown below in Table 2, as well as in FIG. 7, which provides a representation of the mean concentrations of Compound (I-g) over a 24 hour time period after dosing on day 15 for all three tested doses (35 mg/day, 70 mg/day, and 140 mg/day).

TABLE 2

| Dose | Mean Day 1 | Mean | Mean Day 15 | Mean |
|---|---|---|---|---|
| (oral, once | AUC₀₋₂₄ | Day 1 | AUC₀₋₂₄ | Day 15 |
| daily) | (ng * hr/mL) | Cₘₐₓ (ng/mL) | (ng * hr/mL)^{a} | Cₘₐₓ (ng/mL) |
| 35 mg | 160.5 | 11.1 | 1701 | 83 |
| 70 mg | 300 | 19.6 | 2538 | 141 |
| 140 mg | 865 | 54 | 5023 | 353 |

^{a}Day 15 AUCs calculated using imputed 24 hours values.

### Example 7—Phase I Dose Escalation Studies with Compound (I-g)

Compound (I-g) was administered orally to human subjects (n=22) at doses of 35 mg/day, 70 mg/day, 140 mg/day, and 280 mg/day.

In the 35 mg/day cohort (n=3), no dose limiting toxicity was observed and no adverse events at grades 2, 3, or 4 were observed.

In the 70 mg/day cohort (n=4), no dose limiting toxicity was observed. One patient experienced grade 2 adverse events (diarrhea, fatigue, vomiting). One patient experienced a grade 3 adverse event (anemia) that was unrelated to the administration of compound (I-g).

In the 140 mg/day cohort (n=8), no dose limiting toxicity was observed. 50% of the patients experienced grade 2 adverse events and 1 patient experienced a grade 3 adverse event (decreased lymphocyte count). These results do not include one patient in this cohort group who was determined to be non-evaluable and treatment was discontinued on day 1.

In the 280 mg/day cohort (n=7), one patient experienced dose-limiting toxicity and renal failure, and 5 of the patients experienced grade 2 or less adverse events.

### Example 8—Evaluation of Best Percent Change of Plasma PSA from Pre-Treatment Levels in Patients with mCRPC and Subsequent Evaluation of Biomarker Status after Oral Administration of Compound (I-g)

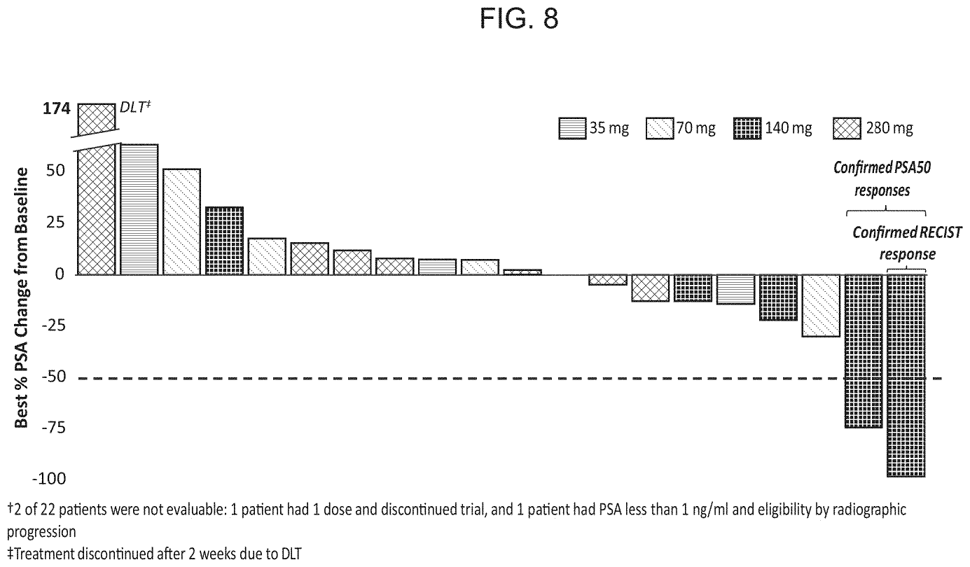

Twenty patients were administered Compound (I-g) orally at doses of 35 mg/day, 70 mg/day, 140 mg/day, or 280 mg/day. The best percent change in plasma PSA from pre-treatment levels for each of the twenty patients is provided in FIG. 8. Patient 19 (second bar from right) and Patient 20 (rightmost bar) had at least a 50% reduction in PSA after treatment with Compound (I-g).

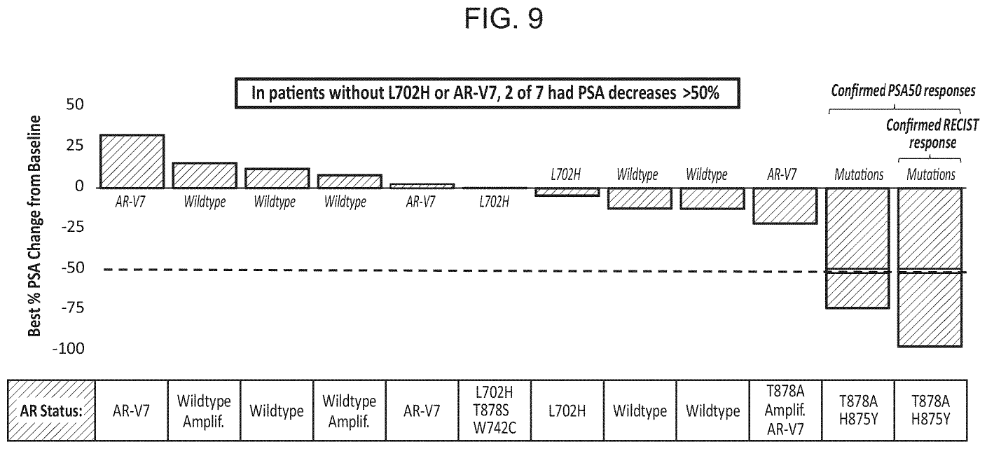

The AR biomarker status of twelve patients who were administered Compound (I-g) orally at a dose greater than or equal to 140 mg/day was evaluated. FIG. 9 shows the AR biomarker status of these 12 patients along with their best percent change in plasma PSA levels. Patients with different AR biomarker status had different responses to treatment with Compound (I-g). For instance, Patient 19 (second bar from right) and Patient 20 (rightmost bar), who both had T878A and H875Y AR mutations, were the only patients in this study who had at least a 50% reduction in PSA after treatment.

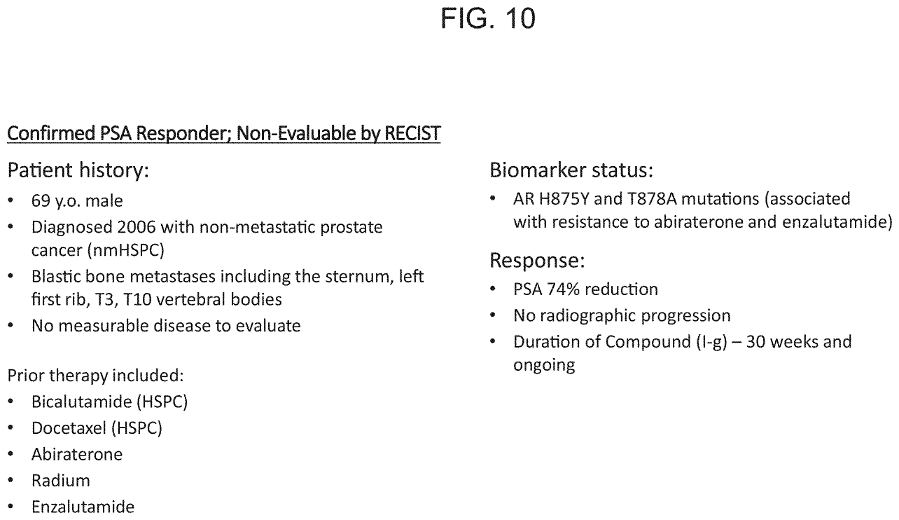

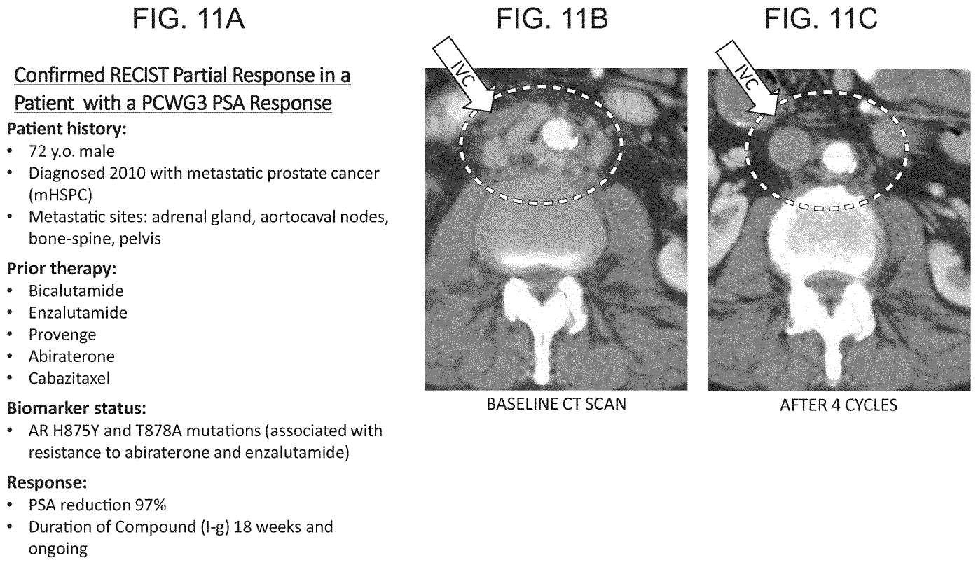

The key features of Patients 19 and 20 are summarized in FIG. 10 and FIG. 11A, respectively. FIG. 11B shows a CT scan of Patient 20's tumor prior to treatment with Compound (I-g). FIG. 11C shows a CT scan of Patient 20's tumor after 4 cycles, showing the RECIST response.

### Example 9—Further Pharmacokinetic Data—Oral Administration of Compound (I-g)

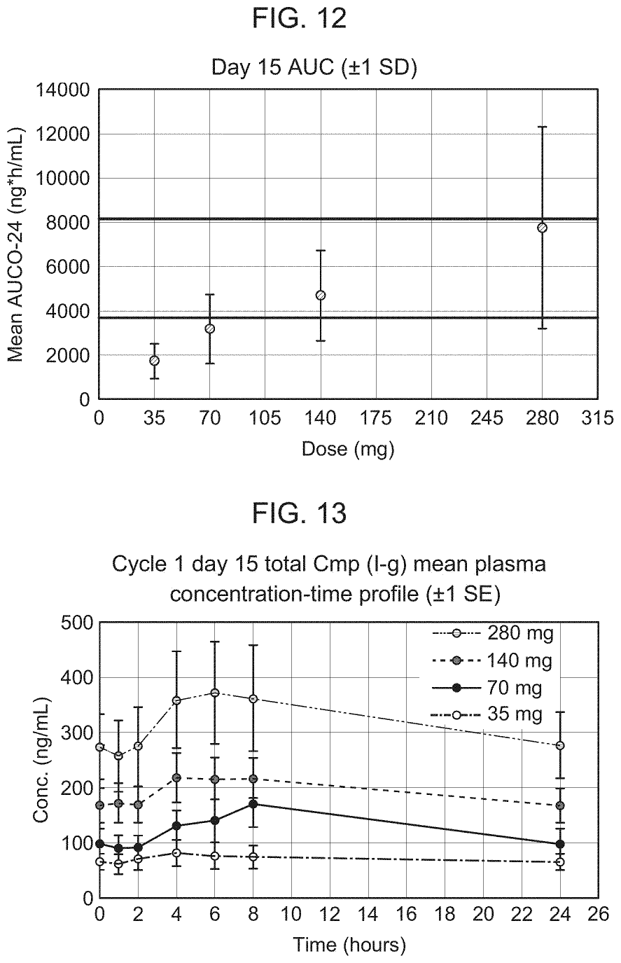

Compound (I-g) was administered orally at a dose of 35 mg/day, 70 mg/day, 140 mg/day, and 280 mg/day. It was observed that treatment with 140 mg/day and 280 mg/day dose of Compound (I-g) enters the preclinical efficacious range associated with tumor growth inhibition. (FIG. 12.) The mean plasma concentrations of Compound (I-g) over a 24 hour time period after dosing on day 15 for all four tested doses (35 mg/day, 70 mg/day, 140 mg/day, and 280 mg/day) are provided in FIG. 13.

### Example 10—AR Biomarker Studies

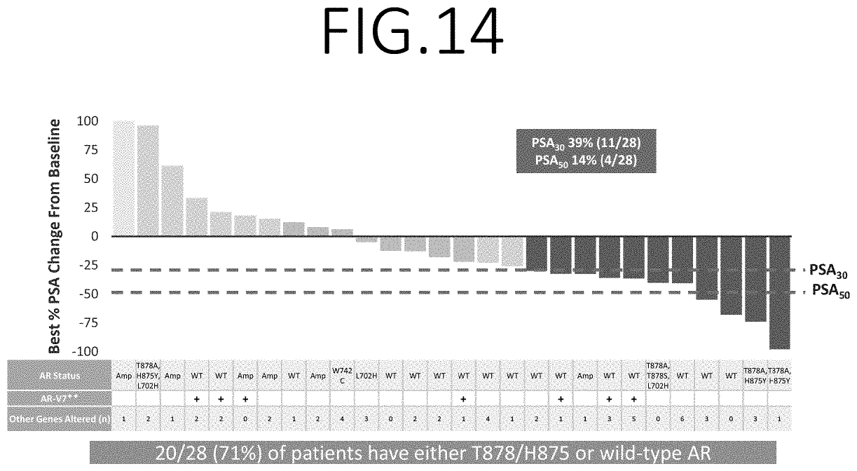

FIG. 14 is a waterfall plot of 28 patients with mCRPC that were administered Compound (I-g). The molecular status of the AR gene present in circulating tumor DNA or circulating tumor cells was determined for each patient. Each bar represents the best percent change in plasma PSA from pre-treatment levels of a single patient. AR-V7 is a splice variant of AR. Amplif. refers to amplification of the AR gene.

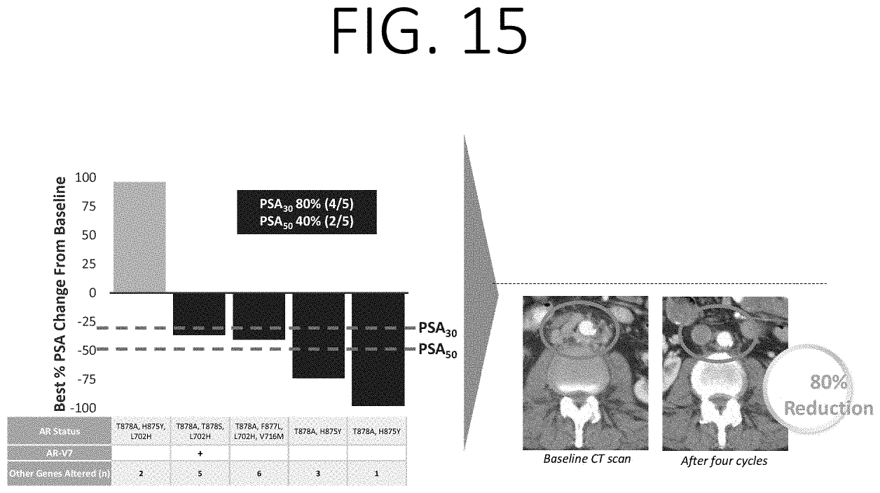

FIG. 15 is a waterfall plot showing the best percent change in PSA test results in 5 patients with mCRPC with AR T878/H875 mutations, after each patient received Compound (I-g), as well as the molecular status of the AR gene present in circulating tumor DNA or circulating tumor cells, respectively, isolated from each patient. Each bar represents the best percent change in plasma PSA from pre-treatment levels of a single patient. AR-V7 is a splice variant of AR. (AR-V7 status was not available for 3 patients.)

PSA levels declined by 30% or more in 80% of the subjects (⅘) even in the presence of significant tumor heterogeneity. A PSA decline of 50% or more was observed in 40% (⅖) of patients. In one patient (pictured), a PSA decline of 80% was observed.

Thus, patients with AR T878/H875 mutations may represent a subgroup of patients that are particularly sensitive to Compound (I-g).

In an ongoing Phase 1/2 clinical trial evaluating the administration of Compound (I-g) for the treatment of mCRPC, the molecular status of the AR gene present in circulating tumor DNA and expression of the AR-V7 splice variant in circulating tumor cells observed prior to Compound (I-g) treatment and the best change in prostate serum antigen (PSA) test from baseline values observed for each patient were determined. The data are summarized in Table 3 (Phase 1) and Table 4 (Phase 2). All phase 1/2 patients included in the tables were evaluable for AR status and had >1 month of prostate-specific antigen (PSA) follow-up. Phase 1 patients shown either received a total dose ≥420 mg or had Compound (I-g) exposures above the minimum efficacious threshold based on nonclinical studies evaluating tumor growth inhibition in mouse prostate cancer xenograft models.

TABLE 3
Phase 1 Patient Biomarker Status

| Dose (mg)/ | Patient | Best PSA | | |
|---|---|---|---|---|
| Schedule | No. | Change | AR Status | AR-V7 |
| 140 QD | 1 | −98.2% | T878A, H875Y | Negative |
| 700 QD | 2 | −74.4% | WT | Positive |
| 140 QD | 3 | −73.7% | T878A, H875Y | Negative |
| 420 BID | 4 | −72.5% | T878S | Negative |
| 140 BID | 5 | −71.9% | WT | TBD |
| 210 BID | 6 | −71.4% | WT | Negative |
| 700 QD | 7 | −60.7% | WT | Negative |
| 420 QD | 8 | −52.7% | WT | Positive |
| 700 QD | 9 | −47.0% | WT | Negative |
| 420 QD | 10 | −40.6% | L702H, V716M, G751C, | Negative |
| | | | F877L, T878A | |
| 315 BID | 11 | −40.3% | WT | Negative |
| 280 BID | 12 | −38.3% | WT | Negative |
| 140 BID | 13 | −38.1% | WT | TBD |
| 630 QD | 14 | −36.5% | T878A, T878S, L702H | Positive |
| 420 QD | 15 | −33.0% | WT | Negative |
| 210 BID | 16 | −32.5% | Y447N, Amplification | Positive |
| 70 QD | 17 | −30.0% | WT | Negative |
| 315 BID | 18 | −29.9% | L702H, Amplification, | Negative |
| | | | AR rearrangement | |
| 700 QD | 19 | −25.6% | WT | Negative |
| 280 BID | 20 | −25.5% | WT | Negative |
| 280 BID | 21 | −22.9% | WT | Negative |
| 630 QD | 22 | −22.5% | WT | Negative |
| 420 BID | 23 | −22.5% | WT | Negative |
| 630 QD | 24 | −22.4% | WT | Positive |
| 315 BID | 25 | −18.4% | WT | Negative |
| 140 QD | 26 | −12.7% | WT | Negative |
| 280 QD | 27 | −12.6% | WT | Negative |
| 280 QD | 28 | −4.7% | L702H | Negative |
| 420 QD | 29 | 6.4% | W742C | Negative |
| 280 QD | 30 | 8.0% | Amplification | Negative |
| 700 QD | 31 | 9.7% | WT | Negative |
| 280 QD | 32 | 11.8% | WT | Negative |
| 280 BID | 33 | 13.1% | WT | Negative |
| 280 QD | 34 | 15.4% | Amplification | Negative |
| 70 QD | 35 | 17.6% | Amplification | Negative |
| 420 QD | 36 | 20.8% | WT | Positive |
| 210 BID | 37 | 22.1% | WT | Negative |
| 420 QD | 38 | 32.2% | Amplification | Negative |
| 140 QD | 39 | 32.6% | WT | Positive |
| 140 BID | 40 | 38.9% | Amplification, | TBD |
| | | | AR_ARHGEF9 | |
| | | | rearrangement | |
| 280 QD | 41 | 61.1% | Amplification | Positive |
| 280 BID | 42 | 72.6% | Amplification | Positive |
| 140 BID | 43 | 84.6% | L702H, Amplification | TBD |
| | | | T878A, H875Y, L702H, | |
| 420 QD | 44 | 96.3% | Amplification | Negative |
| 210 BID | 45 | 101.8% | Amplification | Negative |
| 280 BID | 46 | 461.2% | Amplification | Positive |

WT = wild type;
QD = once a day;
BID = twice a day;
TBD = to be determined

TABLE 4
Phase 2 Patient Biomarker Status

| Dose | | Best | | |
|---|---|---|---|---|
| (mg)/ | Patient | PSA | | |
| Schedule | No. | Change | AR Status | AR-V7 |
| 420 QD | 1 | −95.8% | T878A | Negative |
| 420 QD | 2 | −94.2% | T878A | Negative |
| 420 QD | 3 | −88.7% | Amplification, H875Y | Negative |
| 420 QD | 4 | −88.7% | T878A, W742C | Negative |
| 420 QD | 5 | −86.4% | T878A | Negative |
| 420 QD | 6 | −84.8% | T878A | Negative |
| 420 QD | 7 | −67.3% | L702H | Negative |
| 420 QD | 8 | −61.9% | Amplification, | Negative |
| | | | H875Y, AR | |
| | | | rearrangement, Q825E | |
| 420 QD | 9 | −60.3% | T878A, W742C | Negative |
| 420 QD | 10 | −58.7% | Amplification | Negative |
| 420 QD | 11 | −57.5% | WT | Negative |
| 420 QD | 12 | −57.3% | T878A | Negative |
| 420 QD | 13 | −53.8% | WT | Negative |
| 420 QD | 14 | −53.4% | WT | Negative |
| 420 QD | 15 | −52.1% | WT | Negative |
| 420 QD | 16 | −47.8% | T878A, L702H | Negative |
| 420 QD | 17 | −40.9% | WT | Positive |
| 420 QD | 18 | −40.3% | WT | Negative |
| 420 QD | 19 | −35.4% | WT | Negative |
| 420 QD | 20 | −32.2% | WT | Negative |
| 420 QD | 21 | −31.9% | L702H | Negative |
| 420 QD | 22 | −30.6% | WT | Negative |
| 420 QD | 23 | −29.9% | Amplification | Positive |
| 420 QD | 24 | −28.8% | L702H, H875Y | Negative |
| 420 QD | 25 | −27.7% | WT | Negative |
| 420 QD | 26 | −26.7% | L702H, 5889G | Negative |
| 420 QD | 27 | −26.1% | Amplification | Negative |
| 420 QD | 28 | −24.7% | Amplification, 5532Y | Negative |
| 420 QD | 29 | −24.3% | WT | Negative |
| 420 QD | 30 | −22.8% | Amplification | TBD |
| 420 QD | 31 | −19.0% | WT | Negative |
| 420 QD | 32 | −18.8% | WT | TBD |
| 420 QD | 33 | −16.3% | WT | Negative |
| 420 QD | 34 | −16.2% | WT | Negative |
| 420 QD | 35 | −15.1% | WT | Negative |
| 420 QD | 36 | −15.1% | T878A | Negative |
| 420 QD | 37 | −13.4% | Amplification- | TBD |
| | | | equivocal | |
| 420 QD | 38 | −12.5% | L702H, V716M | Negative |
| 420 QD | 39 | −9.9% | Q799E | Negative |
| 420 QD | 40 | −7.7% | T878A | Negative |
| 420 QD | 41 | −7.6% | L702H, | Negative |
| | | | AR-STARD8 | |
| | | | rearrangement, | |
| | | | Q793E | |
| 420 QD | 42 | −6.2% | WT | Positive |
| 420 QD | 43 | −5.6% | WT | Negative |
| 420 QD | 44 | −5.0% | | Positive |
| 420 QD | 45 | −4.7% | Amplification | Negative |
| 420 QD | 46 | −4.1% | Amplification- | TBD |
| | | | equivocal | |
| 420 QD | 47 | −3.8% | AR Rearrangement | TBD |
| 420 QD | 48 | −3.6% | L702H | Negative |
| 420 QD | 49 | −1.5% | Amplification- | Negative |
| | | | equivocal, AR | |
| | | | rearrangement | |
| 420 QD | 50 | −0.9% | AR-AR deletion | Negative |
| 420 QD | 51 | −0.4% | H875Y | Positive |
| 420 QD | 52 | 0.1% | WT | Negative |
| 420 QD | 53 | 0.5% | WT | Negative |
| 420 QD | 54 | 1.2% | L702H, T878A | Positive |
| 420 QD | 55 | 3.2% | WT | Positive |
| 420 QD | 56 | 4.3% | Amplification | Negative |
| 420 QD | 57 | 7.1% | L702H, Amplification | Negative |
| 420 QD | 58 | 7.2% | WT | Positive |
| 420 QD | 59 | 7.8% | WT | Negative |
| 420 QD | 60 | 9.8% | Amplification | Positive |
| 420 QD | 61 | 11.7% | WT | Positive |
| 420 QD | 62 | 12.2% | Amplification- | Positive |
| | | | equivocal | |
| 420 QD | 63 | 13.3% | L702H, | Positive |
| | | | AR-AR deletion | |
| 420 QD | 64 | 13.9% | WT | Negative |
| 420 QD | 65 | 14.8% | Amplification- | Negative |
| | | | equivocal | |
| 420 QD | 66 | 17.9% | WT | Negative |
| 420 QD | 67 | 19.0% | WT | Negative |
| 420 QD | 68 | 20.5% | WT | Negative |
| 420 QD | 69 | 20.8% | Amplification | Negative |
| 420 QD | 70 | 21.8% | WT | Negative |
| 420 QD | 71 | 21.8% | WT | Negative |
| 420 QD | 72 | 22.6% | WT | Negative |
| 420 QD | 73 | 26.8% | WT | Negative |
| 420 QD | 74 | 29.4% | H875Y | Negative |
| 420 QD | 75 | 32.4% | Amplification | Positive |
| 420 QD | 76 | 37.0% | Amplification | Positive |
| 420 QD | 77 | 42.0% | WT | Negative |
| 420 QD | 78 | 43.9% | L702H, T878A, | Negative |
| | | | Amplification- | |
| | | | equivocal | |
| 420 QD | 79 | 46.9% | WT | Negative |
| 420 QD | 80 | 47.1% | Amplification | Negative |
| 420 QD | 81 | 54.4% | WT | Negative |
| 420 QD | 82 | 57.8% | WT | Negative |
| 420 QD | 83 | 62.9% | WT | Negative |
| 420 QD | 84 | 64.4% | L702H, T878A, | Negative |
| | | | T878S, H875Y | |
| 420 QD | 85 | 67.7% | WT | Negative |
| 420 QD | 86 | 70.5% | Amplification | Negative |
| 420 QD | 87 | 75.3% | T878S, F877L | Positive |
| | | | A688fs*19, | |
| 420 QD | 88 | 78.2% | AR-AR rearrangement | Negative |
| 420 QD | 89 | 79.4% | Amplification | Positive |
| 420 QD | 90 | 89.6% | WT | Positive |
| | | | Amplification, AR | |
| 420 QD | 91 | 100.3% | rearrangement | Negative |
| 420 QD | 92 | 100.7% | E666K | Negative |
| 420 QD | 93 | 142.8% | AR-AR rearrangement | Negative |
| 420 QD | 94 | 162.7% | T878A | Positive |

WT = wild type;
QD = once a day;
TBD = to be determined

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

The methods of the disclosure have been described herein by reference to certain preferred embodiments. However, as particular variations thereon will become apparent to those skilled in the art, based on the disclosure set forth herein, the disclosure is not to be considered as limited thereto.

Unless otherwise defined, all technical and scientific terms used herein have the same meaning as commonly understood by one of ordinary skill in the art to which this disclosure belongs. In the specification and claims, the singular forms also include the plural unless the context clearly dictates otherwise.

It is to be understood that at least some of the descriptions of the disclosure have been simplified to focus on elements that are relevant for a clear understanding of the disclosure, while eliminating, for purposes of clarity, other elements that those of ordinary skill in the art will appreciate may also comprise a portion of the disclosure. However, because such elements are well known in the art, and because they do not necessarily facilitate a better understanding of the disclosure, a description of such elements is not provided herein.

Further, to the extent that a method does not rely on the particular order of steps set forth herein, the particular order of the steps recited in a claim should not be construed as a limitation on that claim.

All patents, patent applications, references and publications cited herein are fully and completely incorporated by reference as if set forth in their entirety. Such documents are not admitted to be prior art to the present disclosure.

1. A method of treating prostate cancer in a subject in need thereof, wherein the prostate cancer comprises at least one somatic AR tumor mutation;
the method comprising administering to the subject a therapeutically effective amount of a compound of Formula (I), 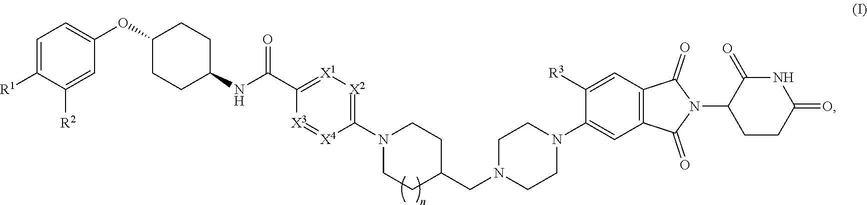
or a pharmaceutically acceptable salt thereof, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH.

2. The method of claim 1, wherein the at least one somatic AR tumor mutation is selected from the group consisting H875X, Q825X, F877X, V716X, T878X, W742X, D891X, M750X, and S889X wherein “X” refers to any amino acid residue other than the wild type residue at that position.

3. The method of claim 1, wherein the at least one somatic AR tumor mutation is selected from the group consisting of T878A, H875Y, H875L, Q825E, W742C, W742L, F877L, T878S, V716M, D891H, M750V, M750T, and S889G.

4. The method of claim 1, wherein the prostate cancer comprises at least two somatic AR tumor mutations.

5. The method of claim 4, wherein the at least two somatic AR tumor mutations are selected from H875X, Q825X, F877X, V716X, T878X, and W742X, wherein “X” refers to any amino acid residue other than the wild type residue at that position.

6. The method of claim 4, wherein the at least two somatic AR tumor mutations are selected from: H875Y, H875L, Q825E, T878A, F877L, V716M, T878S, W742C, and W742L.

7. The method of claim 4, wherein the at least two somatic AR tumor mutations are selected from the following groups of mutations:
T878A, and H875Y;
H875L and Q825E;
T878A, F877L, and V716M;
T878A, M750T, and D891H;
T878S and H875Y;
T878A and T878S;
T878S and W742C; and
W742C and W742L.

8. The method of claim 1, wherein the prostate cancer comprises an amplification of the AR gene.

9. The method of claim 1, wherein the prostate cancer is castrate-resistant prostate cancer.

10. The method of claim 1, wherein the prostate cancer is metastatic prostate cancer.

11. The method of claim 1, wherein R¹ is CN and R² is chloro.

12. The method of claim 1, wherein R³ is hydrogen.

13. The method of claim 1, wherein R³ is fluoro.

14. The method of claim 1, wherein n is 0.

15. The method of claim 1, wherein n is 1.

16. The method of claim 1, wherein each of X¹, X², X³, and X⁴ is CH.

17. The method of claim 1, wherein three of X¹, X², X³, and X⁴ are each CH and the other is N.

18. The method of claim 1, wherein two of X¹, X², X³, and X⁴ are each CH and the other two are each N.

19. The method of claim 1, wherein the compound of Formula (I) is: 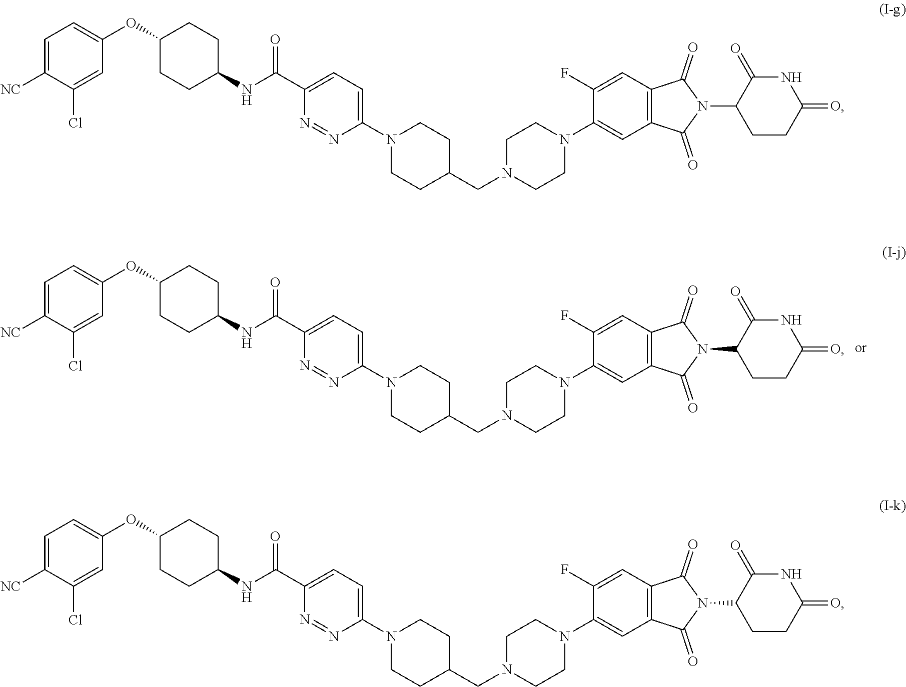
or a pharmaceutically acceptable salt thereof.

20.-33. (canceled)

34. A method of treating prostate cancer in a subject in need thereof, comprising once a day, oral administration of a therapeutically effective amount of a compound of Formula (I), or a pharmaceutically acceptable salt thereof, wherein the compound of Formula (I) is selected from the group consisting of: 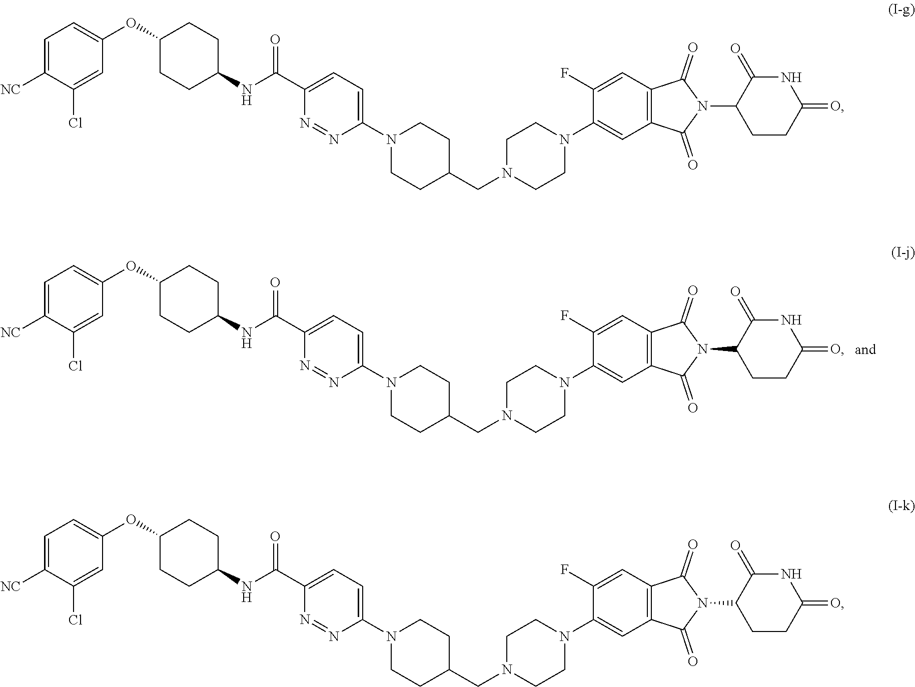
wherein the prostate cancer comprises at least one somatic AR tumor mutation.

35. The method of claim 34, wherein the at least one somatic AR tumor mutation is selected from H875X, Q825X, F877X, V716X, T878X, W742X, D891X, M750X, and S889X wherein “X” refers to any amino acid residue other than the wild type residue at that position.

36. The method of claim 34, wherein the at least one somatic AR tumor mutation is selected from T878A, H875Y, H875L, Q825E, W742C, W742L, F877L, T878S, V716M, D891H, M750V, M750T, and S889G.

37. The method of claim 34, wherein the prostate cancer comprises at least two somatic AR tumor mutations.

38. The method of claim 37, wherein the at least two somatic AR tumor mutations are selected from H875X, Q825X, F877X, V716X, T878X, and W742X, wherein “X” refers to any amino acid residue other than the wild type residue at that position.

39. The method of claim 37, wherein the at least two somatic AR tumor mutations are selected from H875Y, H875L, Q825E, T878A, F877L, V716M, T878S, W742C, and W742L.

40. The method of claim 37, wherein the at least two somatic AR tumor mutations are selected from the following groups of mutations:
T878A and H875Y;
H875L and Q825E;
T878A, F877L, and V716M;
T878A, M750T, and D891H;
T878S and H875Y;
T878A and T878S;
T878S and W742C;
W742C and W742L.

41. The method of claim 34, wherein the prostate cancer comprises an amplification of the AR gene.

42. The method of claim 34, wherein the prostate cancer is castrate-resistant prostate cancer.

43. The method of claim 34, wherein the prostate cancer is metastatic prostate cancer.

44. A method of treating prostate cancer in a subpopulation of prostate cancer subjects, comprising:
selecting a subject with prostate cancer for treatment, wherein the subject's prostate cancer comprises at least one somatic AR mutation; and
administering a therapeutically effective amount of a compound of Formula (I), 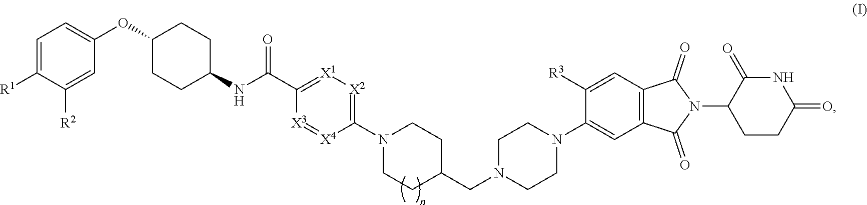
or a pharmaceutically acceptable salt thereof, to the subject, wherein:
R¹ is hydrogen, CN, or C₁-C₆ alkyl;
R² is hydrogen, halo, or C₁-C₆ alkyl;
R³ is hydrogen or halo;
X¹ is CH or N;
X² is CH or N;
X³ is CH or N;
X⁴ is CH or N; and
n is 0 or 1;
provided that at least two of X¹, X², X³, and X⁴ are CH.

45. The method of claim 44, wherein the selected subject's prostate cancer comprises at least one somatic AR tumor mutation selected from H875X, Q825X, F877X, V716X, T878X, W742X, D891X, M750X, and S889X wherein “X” refers to any amino acid residue other than the wild type residue at that position.

46. The method of claim 44, wherein the selected subject's prostate cancer comprises at least one somatic AR tumor mutation selected from T878A, H875Y, H875L, Q825E, W742C, W742L, F877L, T878S, V716M, D891H, M750V, M750T, and S889G.

47. The method of claim 44, wherein the selected subject's prostate cancer comprises at least two somatic AR tumor mutations selected from: H875X, Q825X, F877X, V716X, T878X, and W742X, wherein “X” refers to any amino acid residue other than the wild type residue at that position.

48. The method of claim 44, wherein the selected subject's prostate cancer comprises at least two somatic AR tumor mutations selected from: H875Y, H875L, Q825E, T878A, F877L, V716M, T878S, W742C, and W742L.

49. The method of claim 44, wherein the selected subject's prostate cancer comprises at least two somatic AR tumor mutations selected from the following groups of mutations:
T878A, and H875Y;
H875L and Q825E;
T878A, F877L, and V716M;
T878A, M750T, and D891H:
T878S and H875Y;
T878A and T878S;
T878S and W742C; and
W742C and W742L.

50. The method of claim 44, wherein the somatic AR tumor mutation of the prostate cancer in the selected subject is determined by ctDNA analysis, fluorescent in situ hybridization, immunohistochemistry, PCR analysis, or sequencing.

51. The method of claim 44, wherein the somatic AR tumor mutation of the prostate cancer in the selected subject is determined in a blood sample derived from the subject.

52. The method of claim 44, wherein the somatic AR tumor mutation of the prostate cancer in the selected subject is determined in a solid biopsy derived from the tumor of the subject.

53. (canceled)

54. The method of claim 44, wherein the compound of Formula (I) is: 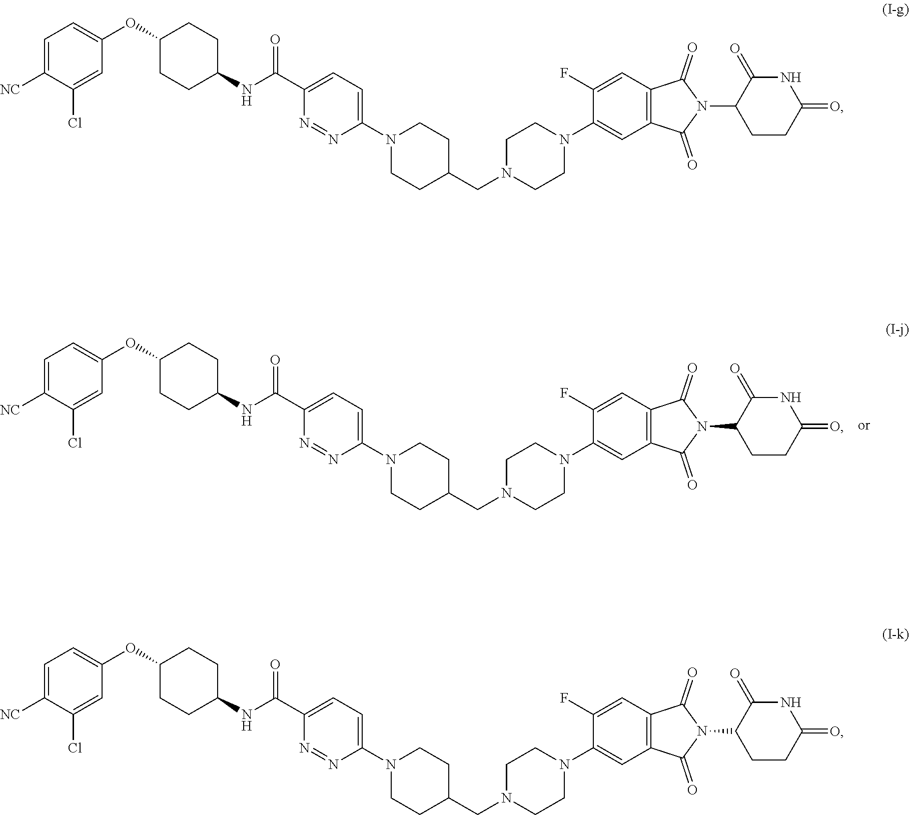
or a pharmaceutically acceptable salt thereof.

55. The method of claim 44, wherein the compound of Formula (I) is:

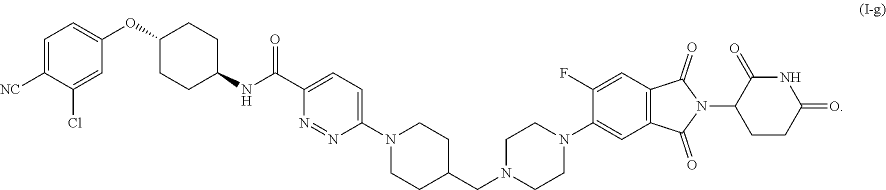 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

56. The method of claim 44, wherein the prostate cancer is castrate-resistant prostate cancer.

57. The method of claim 44, wherein the prostate cancer is metastatic prostate cancer.

58. The method of claim 1, further comprising the administration of at least one additional anti-cancer agent.

59. The method of claim 58, wherein the additional anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

60. The method of claim 58, wherein the additional anti-cancer agent is selected from the group consisting of pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

61. The method of claim 58, wherein the compound of Formula (I) and the additional anti-cancer agent are administered to the subject simultaneously or in temporal proximity.

62.-82. (canceled)

83. A kit comprising:
(a) compound of Formula (I-g): 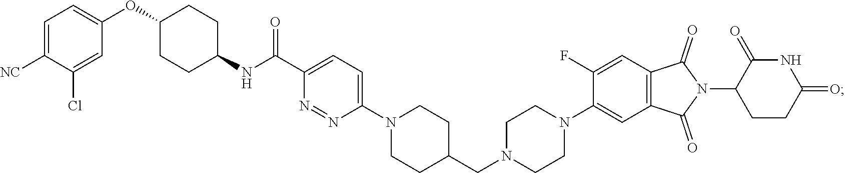 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]
(b) an additional anti-cancer agent; and
(c) instructions for use.

84. The kit of claim 83, wherein the additional anti-cancer agent is a FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, or chemical castration agent.

85. The kit of claim 83, wherein the additional anti-cancer agent is pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, or zolendronate.

86. The method of claim 34, further comprising the administration of at least one additional anti-cancer agent.

87. The method of claim 86, wherein the additional anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

88. The method of claim 86, wherein the additional anti-cancer agent is selected from the group consisting of pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

89. The method of claim 86, wherein the compound of Formula (I) and the additional anti-cancer agent are administered to the subject simultaneously or in temporal proximity.

90. The method of claim 44, further comprising the administration of at least one additional anti-cancer agent.

91. The method of claim 90, wherein the additional anti-cancer agent is selected from the group consisting of FLT-3 inhibitor, androgen receptor inhibitor, VEGFR inhibitor, EGFR TK inhibitor, aurora kinase inhibitor, PIK-1 modulator, Bcl-2 inhibitor, HDAC inhibitor, c-Met inhibitor, PARP inhibitor, CDK 4/6 inhibitor, anti-HGF antibody, IGFR TK inhibitor, PI3 kinase inhibitor, AKT inhibitor, JAK/STAT inhibitor, checkpoint 1 inhibitor, checkpoint 2 inhibitor, PD-1 inhibitor, PD-L1 inhibitor, B7-H3 inhibitor, CTLA4 inhibitor, LAG-3 inhibitor, OX40 agonist, focal adhesion kinase inhibitor, Map kinase kinase inhibitor, VEGF trap antibody, and chemical castration agent.

92. The method of claim 90, wherein the additional anti-cancer agent is selected from the group consisting of pemetrexed, ipilimumab, vorinostat, etoposide, gemcitabine, doxorubicin, vincristine, temozolomide, capecitabine, irinotecan, tamoxifen, anastrazole, exemestane, letrozole, DES, estradiol, estrogen, bevacizumab, goserelin acetate, leuprolide acetate, triptorelin pamoate, medroxyprogesterone acetate, hydroprogesterone caproate, raloxifene, megestrol acetate, carboplatin, cisplatin, dacarbazine, methotrexate, vinblastine, vinorelbine, topotecan, finasteride, arzoxifene, fulvestrant, prednisone, abiraterone, enzalutamide, apalutamide, darolutamide, sipuleucel-T, pembrolizumab, nivolumab, cemiplimab, atezolizumab (Tecentriq), avelumab (Bavencio), durvalumab (Imfinzi), docetaxel (Taxotere), cabazitaxel (Jevtana), mitoxantrone (Novantrone), estramustine (Emcyt), docetaxel, ketoconazole, histrelin, triptorelin, buserelin, cyproterone, flutamide, bicalutamide, nilutamide, pamidronate, and zolendronate.

93. The method of claim 90, wherein the compound of Formula (I) and the additional anti-cancer agent are administered to the subject simultaneously or in temporal proximity.

94. The method of claim 1, wherein the compound of Formula (I) is:

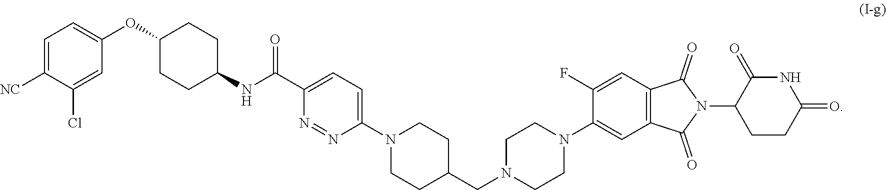 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

95. The method of claim 34, wherein the compound of Formula (I) is:

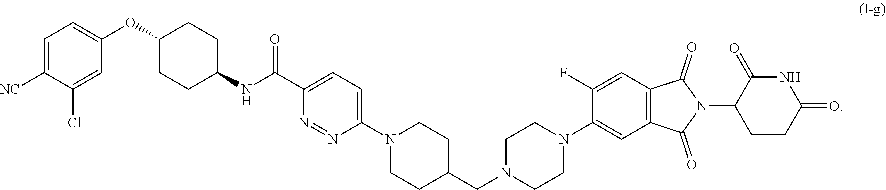 [SMILES: N#Cc1ccc(O[C@H]2CC[C@H](NC(=O)c3ccc(N4CCC(CN5CCN(c6cc7c(cc6F)C(=O)N(C6CCC(=O)NC6=O)C7=O)CC5)CC4)nn3)CC2)cc1Cl]

96. The method of claim 44, wherein R¹ is CN and R² is chloro.

97. The method of claim 44, wherein R³ is hydrogen.

98. The method of claim 44, wherein R³ is fluoro.

99. The method of claim 44, wherein n is 0.

100. The method of claim 44, wherein n is 1.

101. The method of claim 44, wherein each of X¹, X², X³, and X⁴ is CH.

102. The method of claim 44, wherein three of X¹, X², X³, and X⁴ are each CH and the other is N.

103. The method of claim 44, wherein two of X¹, X², X³, and X⁴ are each CH and the other two are each N.
